(19) United States
(12) Patent Application Publication
(10) Pub. No.: US 2025/0001055 A1
(43) Pub. Date: Jan. 2, 2025

(54) BACTERIAL NANOCELLULOSE AND METHOD FOR MAKING THE SAME

(71) Applicant: BIOTRONIK AG, Buelach (CH)

(72) Inventors: Alexander Rzany, Nuernberg (DE); Verena Niopek, Erlangen (DE); Bernhard Hensel, Erlangen (DE)

(21) Appl. No.: 18/709,764

(22) PCT Filed: Nov. 17, 2022
(86) PCT No.: PCT/EP2022/082233

§ 371 Date: May 13, 2024

(30) Foreign Application Priority Data
Nov. 17, 2021 (EP) 21208769.6
Mar. 21, 2022 (EP) 22163189.8

(51) Int. Cl. A61L 31/10; A61L 27/18; A61L 27/34; A61L 31/06; C12N 1/20; C12R 1/00

(52) U.S. Cl. CPC A61L 31/10; A61L 27/18; A61L 27/34; A61L 31/06; C12N 1/20; A61L 2430/20; C12R 2001/00

(57) ABSTRACT

A method for producing a shaped element made of bacterial nanocellulose includes providing a shaped article, providing a growth medium for bacterial nanocellulose comprising Acetobacteraceae bacteria of the genus Komagataeibacter and the species Komagataeibacter hansenii or K. hansenii, preferably in the form of a bacterial suspension, and a nutrient solution for the bacteria, bringing a part of the shaped article into contact with the growth medium for bacterial nanocellulose, and rotating the shaped article to obtain the shaped element made of bacterial nanocellulose. Preferably, rotating the shaped article is carried out at a rotational speed of higher than 10 rpm and maximum 60 rpm and/or wherein the ratio of the bacterial suspension to the nutrient solution is 1:18.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/082233, which was filed Nov. 17, 2022, which application claimed priority from EP application Ser. No. 21/208,769.6, which was filed Nov. 17, 2021, and from EP application Ser. No. 22/163,189.8, which was filed Mar. 21, 2022.

### FIELD OF THE INVENTION

The invention relates to a bacterial nanocellulose and shaped elements made of bacterial nanocellulose and a method for producing the same.

### BACKGROUND

Cellulose can be produced by plants, animals or microorganisms such as bacteria. Bacterial nanocellulose (BNC) is a specific type of cellulose produced by bacteria. Compared to cellulose produced by plants having cellulose fibres with a diameter in the micrometer range, the cellulose fibres of bacterial nanocellulose have a diameter in the nanometer range. Bacterial nanocellulose is of high purity. The size of the fibers depends on the particular bacterial strain and the selected cultivation conditions. BNC fibers synthesized by K. xylinus, for example, have a diameter of 50 nm to 80 nm, which is 100 times thinner than plant cellulose fibers. Bacterial nanocellulose ((C₆H₁₀O₅)ₙ) is a homopolymer consisting of β-D glucose monomers linked by β-1,4-glycosidic bonds. The crystal structure of native cellulose is referred to as cellulose I. Cellulose-forming bacteria produce both cellulose I and cellulose II. Cellulose II is mainly observed when cellulose I is treated with sodium hydroxide solution, resulting in a thermodynamically more stable structure. Cellulose I is distinguished between the crystalline modification Iα and Iβ. Iα is a metastable phase of cellulose I with a triclinic unit cell, while cellulose Iβ is a stable phase with a monoclinic unit cell. BNC has the highest concentration of cellulose Iα (approx. 70%), making it less thermodynamically stable than other types of cellulose, such as plant cellulose. BNC consisting of nanofibers with a diameter smaller than 100 nm can have a water content of about 98% and is therefore referred to as a hydrogel. The high water content can be attributed to the hydrophilicity of the cellulose. The porous geometry allows absorption of liquids and contributes to the hydrophilicity of the cellulose as well. The high crystallinity (60% to 90%) of the BNC results from the ribbon-like arrangement of the cellulose fibers. Hydrogen bonds between the fibrils stabilize the structure and ensure high mechanical strength. The synthesis or incubation time has an influence on the thermodynamic stability of BNC. A longer period of time increases the number of microfibrils and leads to an increase in hydrogen bonding, which results in higher mechanical stability.

Ways of producing BNC are divided into static and dynamic (stirring or shaking) cultivation. Static cultivation is a widely used method. The main goal for all process variants is to achieve the most reproducible quality possible with optimal properties for the respective application. The culture medium is placed in a dish, inoculated with bacteria and cultivated for 5 days to 20 days. After the acclimation phase of the bacteria at the beginning of a cultivation, a first layer formation is visible after about two days. In the following days, the cellulose fleece grows, gaining thickness and compactness. The activity of the bacteria and thus also the growth of the pellicle is limited by the supply of carbon sources in the culture medium. Once this is used up, the initially rapid growth of the fleece stagnates. The shape of the resulting fleece is determined by the geometry of the cultivation vessel and the interface with the surrounding oxygen. In case of a static cultivation, a coherent cellulose fleece is formed, whereas in the case of stirred synthesis, individual, loose pellicles are formed. In contrast, regularly shaped aggregated cellulose pellicles are formed during dynamic cultivation. The shape and structure varies depending on the selected bacterial strain. Investigations by X-ray diffraction show a lower degree of polymerization as well as a lower crystallinity in stirred cultures. For the incubation of BNC for example, stirred tank reactors, airlift reactors, aerosol reactors are known. In addition to the selected incubation method and the synthesis time, the conditions prevailing during cultivation, such as temperature and relative humidity, determine the cellulose yield and properties. In addition, the selected bacterial strain, the composition of the nutrient medium used, and the ratio of bacteria to nutrient medium during inoculation have a significant influence on the properties of the BNC.

For synthesis of cellulose, both gram-negative microorganisms (Gluconacetobacter, Azotobacter, Rhizobium, Pseudomonas, Salmonella, Alcaligenes) and gram-positive ones (Sarcina ventriculi) can be used. The most frequently used bacteria are Gluconacetobacter: Gluconacetobacter xylinus (G. xylinus, also called Acetobacter xylinum) and Gluconacetobacter pasteurianus (G. pasteurianus).

A problem of gram-negative bacteria is that they produce endotoxins. These endotoxins can cause fever in the human body. The layers of bacterial cellulose can contain residual bacteria, which may not be efficiently removed by conventional methods using detergents, such as sodium dodecyl sulphate as described in EP 1 660 670 A.

Materials to be used for medical implants calls for a high standard in terms of purity and reliable physical properties. Thus, it is an object of the present invention to provide bacterial nanocellulose and shaped elements made of bacterial nanocellulose having improved physical properties and a method for making the same.

This objective is solved by a method having the features of claim 1. Preferred embodiments of the respective aspect of the present invention are described below and are stated in the corresponding sub claims.

### SUMMARY OF THE INVENTION

A preferred method for producing a shaped element made of bacterial nanocellulose includes providing a shaped article, providing a growth medium for bacterial nanocellulose comprising Acetobacteraceae bacteria of the genus Komagataeibacter and the species Komagataeibacter hansenii or K. hansenii, preferably in the form of a bacterial suspension, and a nutrient solution for the bacteria, bringing a part of the shaped article into contact with the growth medium for bacterial nanocellulose, and rotating the shaped article to obtain the shaped element made of bacterial nanocellulose. Preferably, rotating the shaped article is carried out at a rotational speed of higher than 10 rpm and maximum 60 rpm and/or wherein the ratio of the bacterial suspension to the nutrient solution is 1:18.

### BRIEF DESCRIPTION OF THE DRAWINGS

FIG. 1 shows an electron microscope picture of bacterial nanocellulose sheet in a top view,

FIG. 2 shows an electron microscope picture of another bacterial nanocellulose sheet in a top view,

FIG. 3 shows an electron microscope picture of the bacterial nanocellulose sheet of FIG. 1 in a side view,

FIG. 4 shows an electron microscope picture of the bacterial nanocellulose sheet of FIG. 2 in a side view,

FIG. 5A shows a photo of a tubular shaped bacterial nanocellulose,

FIG. 5B shows a schematic drawing of the photo of the tubular shaped bacterial nanocellulose of FIG. 5A,

FIG. 6A shows a photo comparing two different shaped elements made of bacterial nanocellulose,

FIG. 6B shows the schematic drawing of the shaped elements made of bacterial nanocellulose of FIG. 6A,

FIG. 7 shows a schematic drawing of a shaped article for a heart valve prosthesis,

FIG. 8A shows a schematic drawing of a transcatheter heart valve prosthesis comprising bacterial nanocellulose a side view,

FIG. 8B shows a schematic drawing of the transcatheter heart valve prosthesis comprising bacterial nanocellulose of FIG. 8A in a side view,

FIG. 9 shows a schematic drawing of a device for producing bacterial nanocellulose,

FIG. 10 shows a detailed schematic depiction of a device for producing bacterial nanocellulose,

FIG. 11 shows a schematic drawing of a stent covered with bacterial nanocellulose,

FIG. 12 shows SEM images plant cellulose fibers and bacterial nanocellulose fibers, produced by K. hansenii,

FIG. 13 shows a molecular structure of the bacterial nanocellulose,

FIG. 14 shows a BNC fleece,

FIG. 15 shows a specimen geometry for uniaxial tensile tests,

FIG. 16 shows a force-elongation curve of a dried rehydrated BNC specimen,

FIG. 17 shows a force-elongation diagram BNC dried with different drying methods,

FIG. 18 shows a flow chart of a standard process for the production of a BNC nonwoven,

FIG. 19 shows force-elongation curves of BNC specimens having different water content,

FIG. 20 shows a flow chart of a standard stabilization process for obtaining stabilized BNC,

FIG. 21 shows electron microscope images stabilized BNC with different stabilizer concentrations,

FIG. 22A-C show electron microscope images of BNC samples stabilized with PEG400,

FIG. 23 shows a plot of the swelling factor for stabilized BNC samples as a function of the stabilizer concentration,

FIG. 24 shows a flow chart of a standard process for manufacturing a BNC tube,

FIG. 25 shows a flow chart of a standard process for manufacturing a stent graft,

FIG. 26 shows a flow chart of a standard process for manufacturing a 3D BNC component,

FIG. 27 shows a flow chart of a standard process for manufacturing locally swellable BNC,

FIG. 28 shows a schematic representation of a pressing device,

FIG. 29 shows a 3D press mold,

FIG. 30 shows a photo of a TAVI prosthesis having a BNC skirt with locally swellable areas,

FIG. 31 shows a diagram showing the fiber volume and density of BNC as a function of cultivation time,

FIG. 32 shows a diagram showing the fiber volume and density of BNC as a function of bacterial solution to nutrient solution.

### DETAILED DESCRIPTION OF THE PREFERRED EMBODIMENTS

The present invention relates to a shaped element of bacterial nanocellulose and to medical implants including the shaped element of bacterial nanocellulose to be implanted into a patient's body and to a method for producing such implants. The description includes bacterial nanocellulose per se and a process for the production of bacterial nanocellulose, dried bacterial nanocellulose, stabilized dried bacterial nanocellulose and locally swellable bacterial nanocellulose, nanocellulose. The use of bacterial nanocellulose, dried bacterial nanocellulose or stabilized dried bacterial nanocellulose in medical implants is also described.

Preferably, the Acetobacteraceae bacteria of the genus Komagataeibacter and the species Komagataeibacter hansenii are Komagataeibacter hansenii with the American Type Culture Collection (ATCC) code 53582.

The nutrient solution may comprise at least one monosaccharide and/or one disaccharide, at least one peptone and yeast extract, and wherein the growth medium has an acidic pH value. The nutrient solution may comprise glucose, peptone, yeast extract, disodium hydrogen phosphate, and citric acid or consists of these. The peptone may be a soybean peptone. The ratio of the bacterial suspension to the nutrient solution may be 1:18. The method may be carried out in an oxygen-containing environment, preferably in air.

The cultivation of the growth medium may be carried out at a temperature between 23° C. and 30° C., for at least 30 h, to obtain bacterial nanocellulose. The cultivation of the growth medium may be carried out for 48 hours to 114 hours at a temperature between 26° C. and 30° C., preferably at a temperature between 26° C. and 28° C. The cultivation may be carried out in the dark.

The rotating the shaped article may be carried out at a rotational speed of maximum 60 rpm. The rotating the shaped article may be carried out at a rotational speed of between 10 and 60 rpm. The shaped article may be made of a polymer. The polymer may not comprise Si—O groups. The polymer may have a polymer backbone containing alternately ketone and ether groups. The polymer may comprise a polyetheretherketone. For example, 40% to 60%, preferably 50%, of a surface of the shaped article may be in contact with the growth medium. The rotating the shaped article may be carried out at a temperature between 23° C. and 30° C., for at least 30 h. The rotating the shaped article is carried out for 48 hours to 114 hours at a temperature between 26° C. and 30° C., preferably at a temperature between 26° C. and 28° C.

The process may further comprise a step of drying the obtained shaped element made of bacterial nanocellulose to obtain a dried shaped element made of bacterial nanocellulose. The step of drying may be carried out in air. The step of drying may be carried out in air during a rotation of the shaped article. The step of drying may be carried out in air during the rotation of the shaped article at a rotational speed of less than 10 rpm.

Prior to the step of drying, an additional step of treating the obtained bacterial nanocellulose with at least one structure stabilizing agent may be carried out to obtain a stabilized shaped element made of bacterial nanocellulose. The at least one structure stabilizing agent comprises or consists of glycerol and/or polyethylene glycol, preferably comprising 5 wt % to 50 wt % glycerol and/or polyethylene glycol.

The method may further comprise a step of treating the obtained bacterial nanocellulose with hydroxide solution before or after the step of drying.

The shaped article is a rod, a rotationally symmetric body or is a medical implant. The shaped article may be covered by a stent, a heart valve prosthesis, a polymer framework, metal framework or metal alloy framework. The shaped article may be removed from the stent, the heart valve prosthesis, the polymer framework, the metal framework or the metal alloy framework.

Also disclosed is a method for producing bacterial nanocellulose comprising the following steps:
preparing or providing a growth medium for bacterial nanocellulose comprising: Acetobacteraceae bacteria of the genus Komagataeibacter and the species Komagataeibacter hansenii or K. hansenii, preferably in the form of a bacterial suspension, and a nutrient solution for said bacteria, wherein the nutrient solution comprises at least one monosaccharide and/or one disaccharide, at least one peptone and yeast extract, and wherein the growth medium has an acidic pH value, and
a cultivation of the growth medium to obtain bacterial nanocellulose.

The Acetobacteraceae bacteria of the genus Komagataeibacter and the species Komagataeibacter hansenii may be Komagataeibacter hansenii with the American Type Culture Collection (ATCC) code 53582.

The nutrient solution may comprise at least one monosaccharide and/or one disaccharide, at least one peptone and yeast extract, and wherein the growth medium has an acidic pH value. The nutrient solution may comprise glucose, peptone, yeast extract, disodium hydrogen phosphate, and citric acid or consists of these. The ratio of the bacterial suspension to the nutrient solution may be 1:18.

The method may be carried out in an oxygen-containing environment, preferably in air.

The cultivation of the growth medium may be carried out at a temperature between 23° C. and 30° C., for at least 30 h, to obtain bacterial nanocellulose. The cultivation of the growth medium may be carried out for 48 hours to 114 hours at a temperature between 26° C. and 30° C., preferably at a temperature between 26° C. and 28° C. The cultivation may be carried out in the dark.

The growth medium may be brought in contact with at least a part of the shaped article. The shaped article may comprise or consist of a polymer. The polymer may not comprise Si—O groups (thus is not a silicone). The polymer may comprise a polymer backbone which contains alternating ketone and ether groups. The polymer may comprise a polyetheretherketone. The shaped article may be a rod, a rotationally symmetric body or is a medical implant, like a stent or heart valve prosthesis.

The process may further comprise a step of drying the obtained bacterial nanocellulose to obtain a dried bacterial nanocellulose. The step of drying may be carried out in air. Prior to the step of drying, an additional step of treating the obtained bacterial nanocellulose with at least one structure stabilizing agent may be carried out to obtain a stabilized bacterial nanocellulose. The at least one structure stabilizing agent may comprise or consist of glycerol and/or polyethylene glycol, preferably comprising 5 wt % to 50 wt % glycerol and/or polyethylene glycol.

The method may further comprise a step of treating the bacterial nanocellulose with hydroxide solution before or after the drying step of drying.

The method may further comprise a step of pressing the bacterial nanocellulose before, during or after the step of drying e.g. by applying a pressure of 2 N/mm² to 40 N/mm², preferably 10 N/mm², to obtain a pressed bacterial nanocellulose. The step of pressing may be carried out for more than 5 min, preferably 15 min. The step of pressing may be carried out at a temperature of between 20° C. and 90° C., preferably 50° C.

Further disclosed is method for producing bacterial nanocellulose is described comprising the steps of:
providing a shaped article,
providing a growth medium for bacterial nanocellulose comprising a bacterial suspension comprising a bacterial nanocellulose producing bacteria and a nutrient solution comprising a monosaccharide and/or disaccharide, a peptone, a yeast extract, wherein the medium has an acidic pH value,
bringing a part of the shaped article into contact with the medium for bacterial nanocellulose producing bacteria, and
rotating the shaped article, preferably with a rotational speed of up to 60 rpm, and
wherein the shaped article is made of a polymer, preferably the polymer does not comprise siloxane groups.

BNC is formed at the interface between air and nutrient medium. During the first days of synthesis, a gelatinous, loosely branched fiber structure is initially formed. With increasing synthesis time, the fiber network becomes increasingly compact and already formed BNC is displaced into the nutrient medium.

As bacterial nanocellulose producing bacteria gram-negative aerobic bacteria (e.g. Gluconacetobacter or Acetobacter) like Gluconacetobacter xylinus (also called Acetobacter xylinum) can be used. In this application preferably Acetobacteraceae bacteria of the genus Komagataeibacter and the species Komagataeibacter hansenii or K. hansenii are used.

The shaped article can be rotated, preferably with a rotation speed of up to 60 revolutions per minute (rpm). Rotational speeds less than approx. 10 rpm result in an inhomogeneous thickness of the bacterial nanocellulose (along the longitudinal axis of the shaped article, e.g. a rod). Rotational speeds higher than approx. 10 rpm result in a homogeneous thickness of the bacterial nanocellulose (along the longitudinal axis of the shaped article, e.g. a rod). The shaped article can be rotatably mounted, preferably in a device as described below. The shaped article can be of arbitrary shape, no symmetry is required for the described method. It may be a rod for tube shaped elements, a rotationally symmetric body or has the shape of a heart valve prosthesis. The rod may have a diameter of 1 mm to 10 mm, preferably 5 mm to 8 mm, most preferably 7.6 mm. The rod may have a length of 5 mm to 200 mm, preferably 20 mm to 90 mm, most preferably 87 mm. The shaped article may be made of a polymer. In principle the polymer can be any chemically inert, mechanically workable but stable and sterilizable polymer. The shaped article can be made of a thermoplastic (polymer). Preferably the polymer does not comprise Si—O groups, namely chemical groups comprising a silicon atom bound to an oxygen atom, such as siloxane groups (Si—O—Si groups) or silanol groups (Si—OH groups). Thus, the shaped article may not be made of a silicone (polysiloxane). The shaped article is for example made of an organic polymer and the organic polymer does not comprise Si—O groups, such as siloxane groups (Si—O—Si groups) or silanol groups (Si—OH groups). Preferably, the shaped article is made of polymer having a polymer backbone containing alternately ketone (R—CO—R) and ether groups (R—O—R). For example, the shaped article is made of a polyether ether ketone (PEEK). PEEK is an organic thermoplastic polymer. PEEK is a chemically inert, mechanically workable but stable and sterilizable polymer.

Bringing a part of the shaped article into contact with the medium for bacterial nanocellulose producing bacteria, can mean that 40% to 60%, preferably 50%, of the surface of the shaped article is in contact with the medium for bacterial nanocellulose producing bacteria. This is favorable as during rotation the part which is not in contact with the medium for bacterial nanocellulose producing bacteria but with oxygen, which enables obtaining an improved bacterial nanocellulose.

The growth medium for bacterial nanocellulose can comprise a bacterial suspension comprising a bacterial nanocellulose producing bacteria and a nutrient solution comprising. The growth medium for bacterial nanocellulose can consist of a bacterial suspension.

The nutrient solution comprises a carbon source as well as peptone and yeast extract. The latter provide nitrogen and ensure good cell growth. The choice of carbon source (glucose, fructose, mannitol, etc.) significantly determines the yield as well as characteristic properties of the cellulose pellicle. The monosaccharide and/or disaccharide acts as carbon source. The monosaccharide and/or disaccharide can be glucose, fructose or sucrose.

The acidic pH value can be obtained by using citric acid. Aerobic, gram-negative bacteria are efficiently fermented at a pH of 3 to 7 and in a temperature range of 25° C. to 30° C. The metabolism of some carbohydrates leads to the side production of gluconic acid, which lowers the pH of the medium and thus has a negative effect on cellulose yield. However, the presence of antioxidants and polyphenolic compounds inhibits the formation of gluconic acid and is realized by adding disodium hydrogen phosphate and citric acid as buffers in the culture medium.

The nutrient solution may consist of glucose, peptone, yeast extract, disodium hydrogen phosphate, citric acid and a solvent. The solvent of the bacterial suspension and/or the nutrient solution may be (purified) water. Preferably, the growth medium for bacterial nanocellulose comprises a bacterial suspension comprising Acetobacteraceae bacteria, preferably of the genus Komagataeibacter and the species Komagataeibacter hansenii or K. hansenii, and a nutrient solution comprising 20 g/l glucose, 5 g/l peptone, 5 g/l yeast extract, 2.7 g/l disodium hydrogen phosphate, and 1.5 g citric acid. The ratio of the bacterial suspension to the nutrient solution may be between 1:16 and 1:20, preferably 1:18.

The peptone acts as nitrogen source. The peptone can be a soy bean peptone.

The method may be carried out in an oxygen-containing environment, preferably air.

The method may be carried out in a dark environment or under red light or yellow light.

The method may be carried out preferably for 48 hours to 114 hours.

The method may be carried out at a temperature of between 26° C. to 30° C., preferably 28° C.

In a further method step, the obtained bacterial nanocellulose can be dried and/or pressed. A (partial) dehydration by drying or pressing changes the morphology of the biomaterial. The drying is preferably carried out at air, optionally with 3 rpm for 24 h. The bacterial nanocellulose can be removed from the shaped article before or after drying.

In order to obtain a swellable bacterial nanocellulose, the obtained bacterial nanocellulose can be conserved by at least one structure-stabilizing substance before drying. Thus, the method can for example comprise a further step of treating the obtained bacterial cellulose with a solution comprising glycerol and/or polyethylene glycol before drying the bacterial nanocellulose.

The method can further comprise a step of rehydrating the dried bacterial nanocellulose. The obtained (dried and/or rehydrated) bacterial nanocellulose can be cut into desired pieces, preferably the cutting may be done using laser cutting, e.g. by CO₂ laser cutting.

The method can further comprise a step of sterilizing the obtained (dried) bacterial nanocellulose.

The method may further comprise a step of treating the obtained bacterial nanocellulose with a lye or an acid in order to remove cell residues and thus the endotoxins that have a toxic effect on the human or animal body. To overcome the problem of endotoxins produced by the gram-negative aerobic bacteria, a purification of the obtained bacterial nanocellulose with sodium hydroxide (NaOH) solution, preferably 0.1 M NaOH solution, can be carried out. This can reduce the content of endotoxins below 0.1 EU/ml (endotoxin units per millilitre).

The shaped article may be covered with a metallic framework, preferably by stent or a Nitinol framework. In this way, a stent graft can be obtained wherein the graft material is the obtained bacterial nanocellulose. Additionally, a positive fit of the metallic framework and the bacterial nanocellulose can be obtained, i.e. the metallic framework is embedded in the bacterial nanocellulose.

According to the aforementioned method, a shaped element made of bacterial nanocellulose can be produced. A shaped element is an element having a (macroscopic) geometrical shape, like a hollow tube. If the obtained hollow tube is for example cut along its longitudinal axis a rectangular piece of bacterial nanocellulose can be obtained as well. A solution merely comprising bacterial nanocellulose fibres is not understood as a shaped element. A shaped element made of bacterial nanocellulose is described herein as well. The shaped element can have a tubular shape, for example has a hollow cylindrical shape. The shaped element can be a planer or bent sheet. This can be for example obtained when a hollow cylinder of bacterial nanocellulose is cut along its longitudinal axis.

The shaped element made of bacterial nanocellulose may have a wall thickness of less than 70 μm, preferably 40 μm to 60 μm. The shaped element may have a length of 5 mm to 200 mm, preferably 20 mm to 90 mm, most preferably 80 mm. The shaped element may have a diameter of 1 mm to 10 mm, preferably 5 mm to 8 mm, most preferably 9 mm, for example in case it is a hollow tube. The shaped element may have the shape of the outer contour of the shaped article, e.g. the outer counter of a heart valve prostheses or a venous valve prosthesis.

The bacterial nanocellulose obtained by the described method has different properties to the conventional bacterial nanocellulose grown at the surface of oxygen-permeable silicone moulds. The tensile strength of the bacterial nanocellulose obtained by the described method is higher (8.40±0.40 N; 5 mm sample width) than the tensile strength of the bacterial nanocellulose obtained at silicone moulds (4.61±1.23 N; 5 mm sample width), the state of the art method (see Example 1). The fibre density of the bacterial nanocellulose obtained by the described method about five times higher than the fibre density of bacterial nanocellulose obtained at silicone moulds.

Also described herein is a medical implant comprising the nanocellulose, preferably obtained by the aforementioned method, having at least one of the properties described above. The medical implant may be a vascular graft, preferably a stent graft; a medical scaffold, preferably a stent; a cardiac pacemaker; a leadless pacemaker; prosthetic valve, preferably prosthetic heart valve, more preferably a transcatheter heart valve prosthesis; or an artificial venous valve comprising the bacterial nanocellulose. The bacterial nanocellulose can be a tissue patch. The bacterial nanocellulose can have the shape of an envelope for cardiac pacemakers or leadless pacemakers.

Also described herein is the use of the bacterial nanocellulose, preferably produced by the aforementioned method for biomedical applications, for vascular grafts, medical implants, medical scaffolds, covers for cardiac pacemakers, leadless pacemakers, prosthetic valves, prosthetic heart valves, artificial venous valves, transcatheter heart valve prosthesis, covered stents or stent grafts, as tissue patches, as drug coatings, for antimicrobial membranes or for biosensors.

It is further disclosed a device for producing bacterial nanocellulose. The device comprises at least one culture vessel for receiving a medium for bacterial nanocellulose producing bacteria, in which the bacterial nanocellulose can be generated and for receiving at least one shaped article. A culture vessel can be adapted to receive more than one shaped article at the same time. However, it is advantageous to have single a culture vessel for receiving only one shaped body, as then the growth of the bacterial nanocellulose can occur without disturbance. The device further comprises at least one rotating unit for rotatably mounting at least one shaped article. The device also comprises at least one shaped article being rotatably mounted on the rotating unit. The rotating unit can be driven by a motor.

The following apparatus (bioreactor) for producing bacterial nanocellulose is disclosed comprising
at least one reactor vessel for receiving and cultivating a growth medium for bacterial nanocellulose and for accommodating one profile of rotation and/or at least one shaped body,
a rotating unit for the rotatable mounting of the at least one rotation profile and/or or shaped body,
at least one rotary profile and/or at least one shaped body, which is rotatably mounted on the rotary unit is rotatably mounted,
at least one drive unit with a geared motor,
at least one rotating unit which is driven by the geared motor,
at least one gear unit for transmitting the motor torque of the geared motor to the at least one rotary profile and/or the at least one molded body,
optionally at least one detection unit for detecting a rotational speed of the at least one rotation profile and/or of the at least one shaped body, preferably comprising at least one Hall sensor,
and optionally at least one evaluation unit and/or control unit of the rotational speed of the at least one rotation profile and/or of the at least one molded body.

The gear unit may comprise at least one shaft, at least one toothed belt and at least one toothed wheel. The apparatus may further comprise at least one detection unit for detecting the rotational speed of the at least one rotation profile and/or of the at least one shaped body. The at least one detection unit may comprise at least one Hall sensor.

It is advantageous that each reactor vessel only receives one rotary profile and/or one shaped body, as then the growth of the bacterial nanocellulose can occur without disturbance (by other rotary profiles and/or other shaped bodies).

(Native and rehydrated) bacterial nanocellulose (obtained from K. hansenii), consisting of nanocellulose fibers with a diameter of 30 nm to 60 nm and/or having a density in the range of 1.100 g/cm³ to 1.500 g/cm³, preferably 1.30±0.10 g/cm³.

Pressed BNC may have a density of between 100 mg/cm³ to 250 mg/cm³. Dried (except freeze drying) BNC may have a density of between 500 mg/cm³ to 1200 mg cm³. Freeze dried BNC has a density of 19 mg/cm³ to 30 mg/cm³. Rehydrated BNC may have a density of 500 mg/cm³ to 600 mg/cm³.

Also stabilized and dried bacterial nanocellulose having a density of between a density of between 1.100 g/cm³ to 1.500 g/cm³, preferably 1.30±0.10 g/cm³, and/or a refractive index of between 1.30 and 1.40 and/or a tensile strength of more than 30 MPa can be obtained. Furthermore, stabilized and dried bacterial nanocellulose having a breaking strength of between 40N to 63 N and/or a tensile strength of more than 30 MPa and/or an elongation at break of between 30% to 45% and/or a F-modulus of between 130 N and 200 N (for a stabilized and dried bacterial nanocellulose sheet having a width of 10 mm and a length of 50 mm) and/or a density of between 1.140 g/cm³ to 1.215 g/cm³ can be obtained.

Also shaped elements made of native, rehydrated or stabilized and dried bacterial nanocellulose having the aforementioned properties can be obtained.

### Example 1—State of the Art

Bacterial Nanocellulose obtained in EP 3 572 043 A1 uses a growth medium comprised of (a) a bacterial suspension containing Acetobacter xylinum (bacterial nanocellulose grown in 25 ml growth medium inside a 50 ml tube, that is suspended by a Turrax) and (b) nutrient solution containing 20 g/l glucose, 5 g/l peptone, 5 g/l yeast extract, 2.7 g/l disodium hydrogen phosphate, and 1.5 g citric acid. (a) and (b) is mixed in a ratio of 1:12. A silicone hose with a stent is immersed in the growth medium. In this growth medium, the bacterial cellulose is formed at typically 26° C. to 30° C. in an incubator over a period of 6 to 8 days. Layer thicknesses of the cellulose in the range of 0.5 to 10 mm or more can be generated.

### Example 2a

This example uses a growth medium comprised of (a) a bacterial suspension containing Acetobacteraceae bacteria of the genus Komagataeibacter and the species Komagataeibacter hansenii or K. hansenii, preferably with the American Type Culture Collection (ATCC) code 53582, and (b) nutrient solution containing 20 g/l glucose, 5 g/l peptone, 5 g/l yeast extract, 2.7 g/l disodium hydrogen phosphate, and 1.5 g citric acid. (a) and (b) is mixed in a ratio of 1:18. The process of Example 2a is done under static conditions. However, this process could be carried out under dynamic (e.g. rotatory conditions like in Example 2b) as well.

The continued existence of an active bacterial culture is ensured by a regular strain maintenance. The strain maintenance of the bacterial strain Komagataeibacter hansenii (ATCC® 53582) ends in a cycle of seven days. In a sterile laboratory vessel made of polypropylene (pyrogen-free) with a capacity of 50 ml is filled with 25 ml of the standard culture medium. The culture medium is then inoculated with 2 ml of a bacterial suspension. This consists of the nutrient medium and a shredded cellulose fleece, which was cultivated which has been cultivated over the past seven days. The mixture of bacterial suspension and nutrient medium is then placed for synthesis at 27° C. and about 90% relative humidity in a cooled incubator. In order to ensure constant conditions prevail during the cultivation, temperature and rel. humidity in the incubator are monitored. According to this, the temperature remains almost constant even when the incubator is opened for a short time, whereas the rel. humidity drops rapidly and then returns to around 90% within approx. 6 hours. to around 90% within approx. 6 h. In general, the selected parameters, such as the duration of cultivation, depend on the geometry of the cultivation on the geometry of the cultivation vessel and the volume of the nutrient solution. In the context of this work the parameters were adapted therefore particularly to the used materials used. The harvested fleece is finally placed for three days on a horizontal shaker in ultrapure water to remove residues of the nutrient medium. The ultrapure water is the ultrapure water is changed after about 8 h each time. The condition of the cellulose fleece after the condition of the cellulose nonwoven after the winding process is referred to as ‘native’ in the following. The sample geometries used in each case are cut out with a CO₂ laser (30 W, Epilog Zing 24, Epilog).

### Example 2b

In this example, the growth medium for bacterial cellulose producing bacteria according to the present invention was prepared as described in Example 1 with the difference that a ratio of bacterial suspension to nutrient solution of 1:18 instead of 1:12 was used. A shaped article made of a polymer having a polymer backbone containing alternately ketone and ether groups, preferably a PEEK rod, was rotatably mounted in the medium for bacterial cellulose producing bacteria. The shaped article, preferably the PEEK rod, was rotated in air with a rotational speed of at least 10 rpm for 3 days at 28° C. Superfluous bacterial nanocellulose was removed every 12 hours.

The obtained bacterial nanocellulose in form of a hollow cylinder mounted on shaped article, preferably the PEEK rod, may be further process according to at least one of the following steps:
a) rinsing with ultrapure water, e.g. for 2 hours, to obtain cleaned bacterial nanocellulose,
b) drying the (cleaned) bacterial nanocellulose, e.g. in air for 24 h with a rotational speed of 3 rpm, to obtain dried bacteria nanocellulose,
c) rehydrating the dried bacterial nanocellulose with ultrapure water,
d) contacting the (dried or rehydrated) bacterial nanocellulose with 0.1M NaOH solution e.g. for 3 days,
e) removing the bacterial nanocellulose from the PEEK rod before or after steps a), b), c) or d),
f) cutting the bacterial nanocellulose into a desired form, e.g. by a CO₂ laser or a scalpel,
g) optionally drying the bacterial nanocellulose,
h) sterilizing the (dried) bacterial nanocellulose.

For a skilled person it is clear that instead of ultrapure water also other highly purified water e.g. distilled water or deionized water can be used. Highly purified water preferably has a resistivity at 25° C. of ≤18.2 MΩ cm (and a total organic content of <10 ppb and an endotoxin content of <0.03 EU/ml). The obtained nanocellulose has a wall thickness of less than 70 μm, preferably 40 μm to 60 μm (depending on the reaction time). Thus, a much thinner wall thickness as in Example 1 is obtained. Compared to the bacterial nanocellulose obtained in example 1 the bacterial nanocellulose according to Example 2 has a higher inner mechanical stability, a higher mechanical strength. The tensile strength of the bacterial nanocellulose obtained in Example 1 was 8.40±0.40 N whereas the tensile strength of the bacterial nanocellulose obtained in Example 2 was 4.61±1.23 N. The bacterial nanocellulose obtained in example 1 has a four times higher density than the bacterial nanocellulose according to Example 2.

### Example 2c

For the cultivation of a cellulose fleece, standard stainless steel trays (e.g. with the dimensions 300 mm×125 mm×60 mm) are used. These are filled with 480 ml of the standard culture medium (also denoted as nutrient solution) containing 20 g/l glucose, 5 g/l peptone, 5 g/l yeast extract, 2.7 g/l disodium hydrogen phosphate and 1.5 g citric acid.) and 40 ml of bacterial suspension are filled. The ratio of bacterial suspension to culture medium is 1:12. The filled dishes are stored for cultivation at 27° C. and for seven days in the Peltier-cooled incubator. After cultivation a 7 mm to 8 mm thick fleece of BNC is harvested. The liquid level of the excess culture medium 5 mm after cultivation, which is a limiting factor in the synthesis of the fleece. However, a subsequent addition of nutrient medium leads to an interruption of the continuous layer formation of the cellulose fleece.

### Example 3—Implant Envelope for Pacemakers

According to this example, an implant envelope was produced from the bacterial nanocellulose, preferably made by the process of example 2, and was used to accommodate a cardiac pacemaker or a leadless pacemaker. Pieces of bacterial nanocellulose were dried. The layers were glued or sutured to one another, using a polymer thread (PTFE, size 5-0) and a 0.3 mm suture needle, to form an implant envelope for receiving the pacemaker. Into this implant envelope made of bacterial nanocellulose the pacemaker can be inserted and the implant envelope can be further closed by gluing or suturing. The pacemaker covered with the implant envelope made of bacterial nanocellulose can be sterilized and can be packaged. The pacemaker covered with the implant envelope made of bacterial nanocellulose can be stored in a dry state. The pacemaker can be rehydrated by a sterile (isotonic) saline solution shortly before implantation. The bacterial nanocellulose used has a wall thickness of less than 70 μm, preferably 40 μm to 60 μm.

### Example 4—Transcatheter Heart Valve Prosthesis

According to this example, components of a transcatheter heart valve prosthesis like the inner and/or outer skirt are made from the bacterial nanocellulose, preferably made by the process of example 2. The leaflets may be made of bacterial nanocellulose or pericardial tissue. A transcatheter heart valve prosthesis is a heart valve prosthesis that is intended for implantation as a replacement of the natural mitral valve. A transcatheter heart valve prosthesis is brought to the implantation site by a catheter system and is anchored there. The anchoring in the vessel wall is implemented by a support structure for the actual heart valve, for example by a metallic mesh having a design and material selection similar to that of a stent, which is therefore also referred to in the following as a stent base body. The stent base body can be self-expanding or can be expanded using a balloon catheter. A transcatheter heart valve prosthesis comprises a stent base body, which can be expanded from a first size, which is configured for minimally invasive insertion, into a functional, second size. The actual heart valve is fixed on this stent base body, wherein said heart valve initially assumes a first shape, which is configured for minimally invasive insertion and which can be expanded, over the course of implantation, into the functional, second shape. The stent base body comprises metallic struts, e.g. nitinol struts. An outer skirt and/or an inner skirt covering the stent base body at least partially can be made of the bacterial nanocellulose and can be fastened to the stent base body (e.g. by gluing or suturing using thread, for example a polytetrafluoroethylene thread), wherein said outer skirt and/or inner skirt is adjoined by the cardiac valve leaflets. Since the bacterial nanocellulose can be processed and stored in the dry state, and since the bacterial nanocellulose can be produced in different layer thicknesses, with different swelling capacities and mechanical strengths, it is possible to construct the entire transcatheter heart valve prosthesis, for example a transcutaneous aortic valve, out of bacterial nanocellulose. The bacterial nanocellulose used has a wall thickness of less than 70 μm, preferably 40 μm to 60 μm.

### Example 5—Covered Stent

According to this example, a stent is covered by bacterial nanocellulose preferably made by the process of example 2. The bacterial nanocellulose used has a wall thickness of less than 70 μm, preferably 40 μm to 60 μm. A stent (which is also referred to as a vascular support) is a medical implant that is inserted into hollow organs, in order to hold these open. The stent is usually a small lattice framework in the shape of a small tube composed of a metal or plastic mesh, which is also referred to as a stent base body in the present case. This stent base body is covered with a layer of bacterial nanocellulose.

### Example 6—Stent Graft

According to this example, an inner and/or outer shell of a stent graft is produced from the bacterial nanocellulose, preferably made by the process of example 2. The bacterial nanocellulose used has a wall thickness of less than 70 μm, preferably 40 μm to 60 μm. A stent graft is the combination of a stabilizing support frame, which is also referred to in the following as the stent, and an artificial blood vessel (vascular prosthesis). The implantation of a stent graft is an endovascular operation. The stent graft is used, in particular, in order to exclude aneurysms from the bloodstream. In the present case, the stent is provided with an inner shell made of bacterial nanocellulose. According to a process of the invention, tubes can be made from bacterial nanocellulose. The (self-expanding or self-expandable) stent is fastened to the inner shell, which was produced using bacterial nanocellulose, e.g. by a surgical suture material or gluing. Strips, which are also tubular and have preferably a width of 1 to 2 cm, are then fastened on the outer side at both ends, e.g. by suturing or gluing. This strip made of bacterial nanocellulose can have a greater swelling capacity as compared to the inner shell and therefore makes it possible to seal leaky points after implantation without substantially increasing the diameter of the implant during implantation.

### Example 7—Vascular Patches

According to this example, a tissue patch, preferably a vascular patch, comprising the bacterial nanocellulose, preferably made by the process of example 2, is disclosed. The bacterial nanocellulose used has a wall thickness of less than 70 μm, preferably 40 μm to 60 μm. In medicine, a tissue patch, preferably a vascular patch, is understood to be a piece of foreign material that is used in surgical procedures to close an unwanted opening. A patch is always used whenever an opening cannot be closed without complications by a simple seam. One example of regular use are heart surgeries in which septal defects, for example, are closed with vascular patches. A patch is also used for the vascular surgical widening of a blood vessel (arterial and venous) or for covering defects on the blood vessels. The patch is sutured into the opened vessel, for example to prevent stenoses caused by seams, or for purposes of widening. In the present case, the patch is produced of the bacterial cellulose treated in the above-described manner. The tissue patch, preferably a vascular patch, may be supported by a bio-degradable or non-biodegradable support structure, e.g. a mesh.

### Example 8

If not stated otherwise the following characteristics refer to BNC obtained in Example 2a:

### Thickness Measurement

To determine the homogeneity of the yield of a cellulose nonwoven, its thickness is determined after the individual process steps. A GT2 Intelligent Series Contact Sensor tactile thickness gauge (Keyence Deutschland GmbH, Germany) is used for this purpose. A hydraulically controlled contact piston with a circular area of 10 mm in diameter exerts a force of 0.3N for 2 s on the material to be measured. The displacement measuring system of the sensor translates the distance between the previously initialized base surface and the measured sample into a thickness value. In addition to the absolute values, the percentage thickness reduction (DR) between the native and rehydrated state of the samples is also indicated is given.

### Mechanical Characterization

BNC is characterized by high mechanical stability and plastic material behavior. For an application in the biomedical field, the integrity of the fiber material after different processing variants is indispensable. The mechanical tensile test is performed in the fully processed, rehydrated state of the samples, unless otherwise specified. A test rig is used, which allows both uni- and biaxial tensile tests. This consists of four drive units, each equipped with a stepper motor and a position encoder and individually controllable. The force is recorded via platform load cells and transferred to the software, which enables both displacement- and time-dependent force measurements. The material specimens are clamped in jaws that are attached to the drive units in a roller guide and are and protrude into a trough filled with ultrapure water. The specimen geometry is developed following DIN EN ISO 527-2 type 1BA. A rectangular geometry with symmetrically arranged recesses corresponding to the shape of a half ellipse is used to ensure continuous loading in the center of the specimen due to the complex fiber composite material. When varying the specimen width, the elliptical recess is scaled accordingly. Prior to measurement, the thickness of the respective specimen is determined tactilely. The recording of the measurement data of the tensile test starts as soon as a preload of 2 g has been reached. The length of the specimen in this state is automatically transferred to the software as the initial specimen length. Unless otherwise specified, the jaws move at a speed of 12 mm/min per drive unit during the measurement until the specimen fails completely. When 1% of the maximum detected force is reached, the measurement is stopped. To characterize the material, the fracture force Fmax and the fracture F_max as well as the slopes in the linear range of the curve are used, on the one hand, at small strains up to 5%, called initial modulus or F-modulus 5%, and, on the other hand, shortly before the failure of the specimen (F-modulus). For a detailed explanation of the complex mechanical behavior of the BNC under uniaxial tensile loading. In addition, the suture retention strength (SRS) is determined as part of the suture tear test. An implant material may require suturing of tissue components or suturing into existing structures, such as in biological heart valve prostheses. The suture pull-out strength provides information on the maximum possible load until a suture is pulled out of the material. For this purpose, the thread is pulled through the material at a distance of 1 mm on the short side of the rectangular specimen measuring 6.5 mm×32 mm and wrapped around a screw. The other side of the specimen is fixed in a clamping jaw as in the mechanical tensile test. The thread has a diameter (D_{Faden}) of 0.1 mm. Before each measurement, the thickness (d_{BNC}) of the puncture is determined tactilely. The seam tear-out strength SRS results from the maximum force measured during the test when the thread is torn out (seam tear-out force, Fₘₐₓ) related to the thickness of the specimen at the puncture point and the diameter of the thread according to equation 3.1.

$$SRS [ \frac{N}{{mm}^{2}} ] = \frac{{F}_{max}}{{D}_{Faden} \cdot {d}_{BNC}} \qquad ( 3.1 )$$

### Water Content and Water Retention Properties

The water content (WC) describes the percentage of water in the fiber network of the BNC. Circular, rehydrated samples (Ø 8 mm) are used for the determination. Water adhering to the surface of the samples is removed by wiping on a grid and then the wet weight (mₘₐₛₛ) is determined using the Excellence Plus XP204 precision balance (Mettler Toledo GmbH). The samples are vacuum freeze dried for 24 h and their dry weight ((m_{trocken}) is determined. The WG is then calculated using equation 3.2 and expressed as a percentage.

$$WG = \frac{{m}_{nass} - {m}_{trocken}}{{m}_{nass}} \qquad ( 3.2 )$$

The water retention value (WRV) describes the ability of fibrous materials to retain water through capillary and adhesive forces within the fibers and their interstices. The determination is carried out according to the standard DIN 53814:1974-10. The samples (Ø8 mm) are centrifuged according to DIN53814: 1974-10) at 2380 U/min and 20° C. for 20 min (centrifuge 5920R, Eppendorf GmbH). After centrifugation, the weight of the samples (m_{zentrifugiert}) is determined. To determine the dry weight (m_{trocken}), the centrifuged samples are dried for 24 h at 100° C. in an oven. The WRV is finally calculated according to equation 3.3.

$$WRV = \frac{{m}_{zentrifugiert} - {m}_{trocken}}{{m}_{trocken}} \qquad ( 3.3 )$$

With the aid of the WRV, it is possible to specify the hornification as a further parameter for characterizing the material structure. This describes the irreversible change of the cellulose fibers due to drying. The fiber network is compressed during drying, resulting in the formation of hydrogen bonds (WBB). The percentage degree of hornification is measured as a decrease in WRV and described by Equation 3.4.

$$Verhormmg = 1 - \frac{{WRV}_{nativ}}{{WRV}_{rehy}} \qquad ( 3.4 )$$

Here, WRVₙₐₜᵢᵥₑ denotes the WRV of the BNC in the native state and WRV_{rehy} denotes the WRV in the dried-rehydrated state.

### Structural Analysis by Scanning Electron Microscopy

The scanning electron microscope (SEM) EVO MA 15 (Carl Zeiss Microscopy AG, Germany) is used to analyse the surface properties. With an excellent resolution and depth of field, the SEM allows structural investigations of the of the BNC down to the nanometer level. From the topography of the surface, the internal microstructure to images of individual fibers, qualitative and quantitative qualitative and quantitative information about the nature of the biomaterial. possible. Since the samples (Ø 10 mm) are scanned under high vacuum, they are vacuum dried beforehand. (2.0×10−1 mbar, Epsilon 1-4 LSCplus, Martin Christ Gefriertrocknungsanlagen GmbH). To generate an electrically conductive surface, the dry samples are then coated with a gold layer under nitrogen atmosphere (Agar Sputter Coater, Plano GmbH, Germany).

### Characteristics of Native Cellulose Nonwovens

During nonwoven synthesis, the morphological properties of the biomaterial are already determined. Various factors influence the cellulose yield and the structure at the micro and nano level. These include the ratio of bacteria to culture medium at constant total volume, the duration of the fleece synthesis and the pH of the culture medium. Based on the standard cultivation, a reproducible nonwoven synthesis results with respect to the pH value when using the medium ATCC 1765. Depending on the cultivation time, the fiber morphology is investigated in the following with the help of SEM images. In addition, the fiber volume and thus the density of the BNC is determined experimentally according to the Archimedean principle using the buoyancy method.

### Determination of the Fiber Diameter by Scanning Electron Microscopy

The dimensions and structure of BNC fibers differ with respect to the bacterial strain. For example, microfibrils of Gluconacetobacter xylinus form fibrillar bands with a width between 40 and 100 nm. Other strains such as Acetobacter bogorensis have fibers on the order of 20 nm. To determine the fiber diameter (FD) of the bacterial strain K. hansenii used in this work, four series of experiments with eight samples each are analysed. The samples of one experimental series differ in their synthesis time (2 d to 9 d). For cultivation, tubes (Sarstedt AG & Co. KG, Ø 17 mm) with a capacity of 15 ml are filled with 1 ml bacterial suspension and 12 ml nutrient medium. After cultivation, the samples are rinsed in ultrapure water, freeze-dried and examined by SEM. For the quantitative analysis, the images are first converted to gray scale images (ImageJ software) to subsequently adjust the contrast and detect structural boundaries (threshold function: threshold ISO 50%). After digitally extracting the desired fiber, its diameter is measured using the software's length measurement tool. To obtain representative values for the diameter analysis, ten different fibers are measured for each sample. The mean value of the diameter of ten fibers per sample is shown in Tab. 3.2. Ten measurements are also performed per fiber. From the recordings and the determined values for the FD, it is immediately evident that the FD is not dependent on the cultivation time. The thinnest fibers have a diameter of about 30 nm and were found both in samples with a very short synthesis time and in samples cultivated for longer than one week.

Overall, the results show that the thinnest fibers on the sample surface in each case can be classified in a range of 30 nm to 60 nm. However, it is often not possible to clearly define whether the fiber under consideration is a single fiber or a fiber composite. In addition, undefined agglomerations of fibers can occur during freeze drying.

The samples of test series V4 are additionally washed in 0.1 mol sodium hydroxide solution before rehydration in order to remove any endotoxins present.

TABLE 3.2
Fiber diameters of three test series of native BNC (V1-
V3) and one test series washed with sodium hydroxide solution
(V4). One sample is analysed per cultivation period, in
which the mean value is formed from ten fibers.

| Cultivation | V1 | V2 | V3 | V4 |
|---|---|---|---|---|
| time | in nm | in nm | in nm | in nm |
| 2 d | 61.0 ± 6.4 | 48.0 ± 4.6 | 34.3 ± 2.4 | 60.6 ± 4.3 |
| 3 d | 58.2 ± 3.9 | 39.2 ± 2.4 | 33.1 ± 2.6 | 54.2 ± 5.7 |
| 4 d | 55.8 ± 2.8 | 48.5 ± 2.9 | 53.6 ± 1.8 | 50.7 ± 3.3 |
| 5 d | 56.3 ± 1.6 | 46.2 ± 1.4 | 55.3 ± 2.3 | 53.3 ± 3.5 |
| 6 d | 56.9 ± 3.2 | 46.3 ± 2.6 | 55.8 ± 3.3 | 50.1 ± 5.4 |
| 7 d | 40.1 ± 2.1 | 34.3 ± 3.5 | 44.9 ± 8.1 | 48.4 ± 4.2 |
| 8 d | 59.0 ± 2.1 | 36.1 ± 3.0 | 40.9 ± 2.4 | 45.7 ± 3.4 |
| 9 d | 56.3 ± 2.8 | 34.9 ± 2.4 | 35.4 ± 3.6 | 49.9 ± 3.7 |

### Determination of Fiber Volume and Density by Buoyancy Weighing

The characteristics of nonwoven thickness and dry mass, as well as fiber volume and density, are determined as a function of the duration of nonwoven synthesis and the ratio of bacterial volume to nutrient medium. The cultivation time is varied between 2 d and 10 d, with a bacteria to nutrient medium ratio of 1:12. This ratio is further changed to 1:6 or 1:96 at constant total volume (520 ml) and cultivation time of 7 d.

The specimens used have the dimensions 3.5 cm×3.5 cm. For the determination of the nonwoven thickness, this is first measured tactilely. The wet specimens, stored in ultrapure water, are weighed (mw) in liquid of known density in the specially constructed apparatus. Ultrapure water is used as the liquid, and its temperature is determined for density determination. Subsequently, the samples are freeze-dried for 48 h and their dry mass (m_{T}) is determined. The difference of the two weighings (m_{T}−m_{W}) represents the mass of the displaced liquid (Archimedes' principle). Taking into account the density of the liquid (ρ_{W}), this gives the volume of the displaced liquid and thus the volume of the displacement body, which in this case corresponds to the volume of the cellulose fibers (V_{Fasern}):

$${V}_{Fasern} = \frac{{m}_{T} - {m}_{W}}{{ρ}_{W}} \qquad ( 3.5 )$$

For the density of the BNC (BNC) applies accordingly:

$${ρ}_{BNC} = \frac{{m}_{T}}{{V}_{Fasern}} = \frac{{m}_{T} \cdot {ρ}_{W}}{{m}_{T} - {ρ}_{W}} \qquad ( 3.6 )$$

The fleece thickness and the dry mass increase steadily during synthesis with longer cultivation time. Only after 6 d and 7 d, respectively, no significant change takes place with respect to the web thickness. In contrast, the dry mass also increases up to a duration of 10 d. This indicates that with longer synthesis time, more glucose is metabolized by the bacteria, generating more cellulose fibers. Accordingly, the fiber network becomes denser over time. This is also confirmed by SEM images of samples with different synthesis times. Compared to a synthesis of 2 d, the fiber network is more densely packed in a 10 d cultured sample. The ratio of bacterial volume to nutrient medium does not show significant differences with respect to nonwoven thickness and dry mass. There is a tendency for both characteristic values to be slightly higher for larger ratios (1:6) than for smaller ratios (1:96). The small differences are due to the proliferation of the bacterial population by cell division. The growth curve of microorganisms is characterized by an exponential growth. The bacteria are optimally adapted to the nutrient medium and the environment and multiply at their maximum division rate. Based on the results, a doubling time of the bacteria within one day or a few hours can be assumed. The BNC hydrogel analysed here is a fiber composite consisting of water and cellulose fibers. The volume fraction of the total volume occupied by the cellulose fibers in this composite is referred to as fiber volume (FV) in the following. The fiber volume per cm³ remains almost constant at 0.8% up to a cultivation time of 6 d. Up to a synthesis time of 10 d, the fiber volume increases to 1.5%. This is consistent with the hypothesis of a larger fiber number with approximately constant fiber diameter with increasing synthesis time.

Changing the ratio of bacterial volume to nutrient medium has no effect on the fiber volume. The density of the BNC can also be derived from the fiber volume and the dry mass (equation 3.6). On average, the measurements yield a density of (1.3±0.1) g/cm³ for the BNC of the bacterial strain K. hansenii (ATCC 53582).

### Determination of the Number of Fibers

Based on the knowledge about the density of the BNC and the fiber volume per cm³ a determination of the number of fibers N per cm³ is possible. First, the volume of a fiber V_{F} is determined. It is assumed that the fiber has a cylindrical base. The base area is assumed with a radius of 30 nm, derived from the determined fiber diameter 30 nm to 60 nm. 10 μm are assumed for the length of a fiber. The volume of a fiber thus corresponds to V_{F}=2.83·10⁻¹⁴ cm³. The number of fibers per cm³ is determined from the quotient of the fiber volume (F_{V}) per cm³ and the volume of a fiber V_{F}. Using the example of a cellulose nonwoven with a synthesis time of 7 d, the fiber number is N per cm³:

$$N = \frac{FV}{{V}_{F}} = \frac{1.13 \cdot {10}^{- 2}}{2.83 \cdot {10}^{- 14} {cm}^{3}} = 3.99 \cdot {10}^{11} \frac{1}{{cm}^{3}} \qquad ( 3.7 )$$

Alternatively, the number of fibers can be derived by calculating the mass of a fiber (m_{F}), taking into account the determined density (ρ_{BNC}). The mass of a fiber is therefore independent of the cultivation time:

$${m}_{F} = {ρ}_{BNC} \cdot {V}_{F} = 1.29 \frac{g}{{cm}^{3}} \cdot 2.83 \cdot {10}^{- 14} {cm}^{3} = 3.65 \cdot {10}^{- 14} g \qquad ( 3.8 )$$

Using the dry mass of a 7 d synthesized BNC sample (m_{T}(7 d)) relative to the total volume of the sample in cm³, this approach yields N per cm³ for the number of fibers:

$$N = \frac{{m}_{T} ( 7 d )}{{m}_{F}} = \frac{14.88 \cdot {10}^{- 3} \frac{g}{{cm}^{3}}}{3.65 \cdot {10}^{- 14} g} = 4.08 \cdot {10}^{11} \frac{1}{{cm}^{3}} \qquad ( 3.9 )$$

In summary, these considerations show that there are about 1011 fibers in one cubic centimeter of the fiber composite BNC. To classify this result, a comparison to another complex fiber composite material with similar geometric dimensions of the fibers is used. A comparable magnitude of 1012 fibers per cubic centimetre was determined by Tautz (2008) for asbestos fibers, which have geometric dimensions similar to BNC fibers, with a fiber diameter of Ø100 nm and an average length of 1-3 μm.

### Processing and Aftertreatment of Cellulose Nonwovens

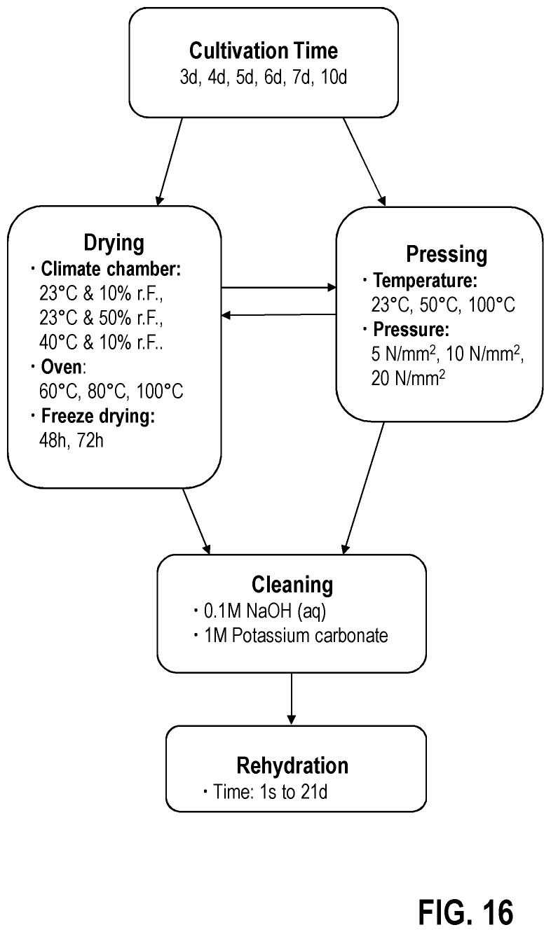

The processing of the BNC following the cultivation is divided into different process variants (see FIG. 16). During cultivation, the duration of tissue synthesis is investigated. Thickness reduction of native nonwovens is achieved by different methods of drying or pressing. A combination of drying and pressing is also in focus. Furthermore, the influence of cleaning for endotoxin removal as well as the rehydration time will be explained. The analytical methods are carried out after rehydration.

### Cultivation

As explained, the thickness of a native cellulose nonwoven and the associated dry mass increases with longer cultivation time. This raises the question to what extent this parameter influences the properties in the dried-rehydrated state.

The cellulose nonwovens are synthesized for 3 d to 10 d. Drying is then carried out in a climatic chamber at 23° C. and 50% relative humidity for 72 h. The cellulose nonwovens are then dried in a drying oven. For a detailed explanation of the drying methods. Finally, rehydration is carried out for 24 h. It can be clearly seen that even in the rehydrated state, the thickness increases with increasing cultivation time (Table 3.3). This is consistent with the increased fiber volume fraction and dry mass of the native samples.

TABLE 3.3
Nonwoven thickness native and dried-rehydrated, water content and water
retention capacity as a function of cultivation time (n = 24, N = 2).

| | | Nonwoven thickness | | | | |
|---|---|---|---|---|---|---|
| Cultivation | | in mm | | WG | WRV | Hornification |
| time | | nativ | rehydriert | in % | in % | in % |
| 3 | d | 2.9 ± 0.5 | 0.09 ± 0.03 | 89.2 ± 8.7 | 379.9 ± 174.9 | 64.2 |
| 4 | d | 4.9 ± 0.4 | 0.13 ± 0.04 | 94.7 ± 3.1 | 234.4 ± 81.5 | 77.9 |
| 5 | d | 6.2 ± 0.6 | 0.17 ± 0.03 | 90.5 ± 1.7 | 233.3 ± 45.6 | 78.0 |
| 6 | d | 7.4 ± 0.8 | 0.24 ± 0.09 | 86.9 ± 3.3 | 207.1 ± 30.4 | 80.4 |
| 7 | d | 7.3 ± 0.4 | 0.26 ± 0.05 | 88.0 ± 3.1 | 133.5 ± 9.7 | 87.4 |
| 10 | d | 7.7 ± 0.5 | 0.30 ± 0.09 | 88.0 ± 2.2 | 189.9 ± 23.3 | 82.1 |

With increasing mass of cellulose fibers, the water content therefore decreases, since the number of fibers in the volume is greater and proportionally fewer interstitial spaces are available for water storage. At the same time, the WRV decreases and the hornification increases, which indicates that more intermolecular WBBs are formed due to the more densely packed fiber structure. This assumption is confirmed when considering the mechanical properties of different cultivation times. The breaking strength increases significantly with increasing synthesis time, whereas the elongation at break remains almost unchanged. This indicates that more load-bearing fibers are present with longer cultivation time, which, however, allow the same macroscopic elongation of the samples. In addition, a stiffer material behavior is evident, not only when considering the slope before the failure point, but also particularly pronounced at the beginning of loading at small strains (Table 3.4). In combination with the increasing hornification with longer cultivation time, this indicates the increased, stabilizing formation of intermolecular WBB.

In addition, it is striking that after seven days of cultivation, fiber synthesis stagnates, which is due to the limited availability of carbon sources in the nutrient medium. The nutrient medium is used up to a supernatant of about 100 ml. In addition, the mechanical strength of the nonwoven no longer increases significantly. For this reason, nonwovens with a synthesis time of seven days are used as standard for subsequent investigations. Subsequent addition of nutrient medium to provide carbon sources again would lead to an interruption of the continuous layer formation, so that no coherent fleece is formed.

TABLE 3.4
Mechanical properties of the failure point and force
response at small strains up to 5% as a function
of cultivation time (n = 60, N = 2).

| | | breaking | breaking | | |
|---|---|---|---|---|---|
| cultivation | | strength | elongation | F-Modul | F-Modul_{5%} |
| time | | in N | in % | in N | in N |
| 3 | d | 13.9 ± 3.8 | 40.8 ± 9.7 | 41.6 ± 10.2 | 57.3 ± 14.6 |
| 4 | d | 25.7 ± 3.2 | 41.7 ± 7.7 | 70.6 ± 14.9 | 108.5 ± 21.4 |
| 5 | d | 38.3 ± 2.8 | 38.4 ± 6.0 | 108.8 ± 21.9 | 191.2 ± 21.0 |
| 6 | d | 52.6 ± 4.9 | 40.5 ± 5.5 | 139.8 ± 15.8 | 223.3 ± 27.4 |
| 7 | d | 55.5 ± 4.3 | 39.4 ± 4.9 | 152.8 ± 18.7 | 272.9 ± 29.4 |
| 10 | d | 58.1 ± 6.2 | 37.1 ± 2.6 | 154.3 ± 13.7 | 283.4 ± 18.1 |

### Drying

Drying of the native cellulose nonwovens is an essential step in the processing. Besides the thickness reduction, the physical properties of the biomaterial are significantly influenced. Depending on the selected drying method, dehydration leads to structural changes in the fiber network. In the following, a distinction is made between drying in the climatic chamber (KS), oven drying (O) and freeze drying (GT). Climatic cabinet drying is characterized by low temperatures and controllable ambient air humidity. In the oven, on the other hand, drying is carried out at high temperatures. In both variants, the samples to be dried are placed between filter paper and weighted down with a grid and stainless steel square pieces (total weight 300 g). This reduces the wrinkling induced by drying and achieves a planar specimen surface. In a freeze-drying step the samples are first frozen on a heat-conductive support at normal pressure. Then, by reducing the pressure to 0.07 mbar, the frozen water changes to the gaseous state. By reaching the sublimation pressure, the phase transition occurs. Finally, the temperature is gradually increased up to 20° C. After drying, all samples are rehydrated for 24 h at 37° C. in ultrapure water. The percentage thickness reduction of the samples by different drying methods does not show any significant differences between climatic chamber and oven drying (Tab. 3.5). Based on the native initial thickness, both methods achieve a remarkable thickness reduction (DR) of about 98%. In contrast, after freeze-drying, only a reduction of the thickness by about half of the initial state is shown. This illustrates the structure-preserving character of freeze-drying by sublimation.

TABLE 3.5
Thickness as a function of different drying
methods (n = 60, N = 2).

| | native | dried | rehydrated | DR |
|---|---|---|---|---|
| sample | in mm | in mm | in min | in % |
| KS (23° C., 24 h, | 7.84 ± 0.48 | 0.15 ± 0.04 | 0.16 ± 0.01 | 97.96 |
| 10%) | | | | |
| KS (40° C., 24 h, | 7.84 ± 0.48 | 0.12 ± 0.03 | 0.15 ± 0.01 | 98.04 |
| 10%) | | | | |
| KS (23° C., 72 h, | 8.37 ± 0.34 | 0.13 ± 0.03 | 0.16 ± 0.01 | 98.07 |
| 50%) | | | | |
| O (60° C., 24 h) | 8.03 ± 0.57 | 0.15 ± 0.04 | 0.16 ± 0.01 | 97.97 |
| O (80° C., 24 h) | 7.28 ± 1.19 | 0.13 ± 0.03 | 0.16 ± 0.01 | 97.85 |
| O (100° C., 12 h) | 8.23 ± 0.48 | 0.12 ± 0.02 | 0.15 ± 0.01 | 98.21 |
| O (100° C., 24 h) | 7.78 ± 0.25 | 0.13 ± 0.04 | 0.14 ± 0.01 | 98.20 |
| GT (48 h) | 7.92 ± 0.45 | 7.44 ± 0.84 | 4.04 ± 0.99 | 48.95 |
| GT (72 h) | 8.50 ± 0.21 | 7.41 ± 0.37 | 6.68 ± 0.31 | 21.44 |

TABLE 3.6
Water content, water retention capacity and hornification as a function
of different drying methods (n = 24, N = 2).

| | WG | WRV | hornification |
|---|---|---|---|
| sample | in % | in % | in % |
| Nativ | 98.1 ± 0.2 | 1060.7 ± 108.4 | — |
| KS (23° C., 24 h, 10%) | 72.6 ± 5.8 | 127.1 ± 7.9 | 88.0 |
| KS (40° C., 24 h, 10%) | 73.8 ± 5.7 | 124.4 ± 8.2 | 88.3 |
| KS (23° C., 72 h, 50%) | 71.6 ± 4.5 | 133.5 ± 9.7 | 87.4 |
| O (60° C., 24 h) | 77.1 ± 6.0 | 117.0 ± 13.9 | 89.0 |
| O (80° C., 24 h) | 62.3 ± 7.4 | 114.2 ± 4.6 | 89.2 |
| O (100° C., 12 h) | 68.7 ± 8.9 | 92.6 ± 6.8 | 91.3 |
| O (100° C., 24 h) | 56.8 ± 3.8 | 104.3 ± 21.5 | 90.2 |
| GT (48 h) | 96.6 ± 0.5 | 762.7 ± 42.0 | 28.1 |
| GT (72 h) | 98.4 ± 0.1 | 995.2 ± 141.6 | 6.1 |

Also with regard to the WG and WRV results, the GT shows comparable values to native samples, whereas in the case of climatic chamber and oven drying, the WG and WRV decrease significantly with increasing temperature during drying. It is assumed that at higher temperatures a stronger compression of the fibers occurs, accompanied by an increasing agglomeration of these. The very dense fiber network during oven drying (100° C.), compared to climatic cabinet or freeze drying, is evident when observing the topography by SEM images. This is also confirmed by the analysis of the mechanical properties. A noticeable increase of the initial modulus at higher drying temperature is observed. Elongation at break and force modulus do not show any significant differences, whereas the breaking force, compared to native specimens, increases significantly due to the drying process (Tab. 3.7). The microstructure of the fibers provides an explanation for the material behavior after drying. These are composed of glucose molecules, each of which has three free hydroxyl groups. The fibrils are thus able to form inter- and intramolecular WBB with neighboring glucan chains as well as with water molecules. During drying, the removal of water molecules releases additional hydroxyl groups, which again form WBB with free, neighboring OH groups. The removal of water thus leads to a densification of the fibers accompanied by a collapse of the pore structure. The experimental determination of the material density of the BNC after different drying methods confirms this assumption. The induced hornification of the material is predominantly irreversible even after complete rehydration. Accordingly, the formation of additional WBB primarily results in a stiffer material behavior at low loads as well as a slight increase in the breaking strength.

TABLE 3.7
Mechanical properties of the failure point and
force response at small strains up to 5% depending
on different drying methods (n = 25, N = 2).

| | breaking | elongation | | F- |
|---|---|---|---|---|
| | force | at break | F-Modul | Modul_{5%} |
| | in N | in 1% | in N | in N |
| Nativ | 31.7 ± 7.7 | 33.9 ± 7.9 | 191.0 ± 36.8 | 6.9 ± 5.6 |
| KS (23° C., 10%) | 48.2 ± 6.4 | 37.6 ± 3.6 | 151.9 ± 10.4 | 248.3 ± 14.3 |
| KS (40° C., 10%) | 48.3 ± 6.2 | 38.1 ± 4.1 | 146.2 ± 7.9 | 263.6 ± 17.7 |
| KS (23° C., 50%) | 55.4 ± 6.1 | 38.6 ± 2.7 | 158.1 ± 13.9 | 281.5 ± 16.7 |
| O (60° C., 24 h) | 56.3 ± 4.1 | 36.4 ± 2.5 | 174.4 ± 9.9 | 322.4 ± 25.6 |
| O (80° C., 24 h) | 54.8 ± 3.7 | 37.2 ± 2.3 | 143.5 ± 9.7 | 332.0 ± 18.6 |
| O (100° C., 12 h) | 56.2 ± 4.3 | 36.1 ± 1.5 | 162.3 ± 10.6 | 377.1 ± 25.9 |
| O (100° C., 24 h) | 57.4 ± 5.2 | 32.9 ± 1.7 | 158.0 ± 8.4 | 424.2 ± 25.0 |
| GT (48 h) | 42.3 ± 4.8 | 33.8 ± 3.7 | 183.8 ± 24.2 | 31.3 ± 14.4 |
| GT (72 h) | 48.0 ± 3.8 | 42.1 ± 3.9 | 183.9 ± 14.1 | 13.8 ± 9.5 |

The results of the seam tear-out strength show, independent of the chosen drying method, a maximum force of about 5 N until the thread is torn out. Since the thickness of the specimen is indirectly proportional in the calculation of the seam tear-out strength, a higher strength is obtained for oven-dried compared to freeze-dried specimens. Regardless of the thickness of the specimens, the maximum seam tear strength does not show any significant differences. Accordingly, it can be assumed that the compactness of the fiber network of different BNC nonwovens is comparable, so that almost identical values are obtained with respect to the seam tear-out force.

### Pressing

As an alternative method to drying, a pressing process for initial dehydration in the native state is highlighted below. Here, too, a thickness reduction with accompanying structural change of the biomaterial is achieved, which is desirable for an application in the biomedical field. In particular, a combination of drying and pressing is investigated. After rinsing the native cellulose fleece, a hand lever press (LaboPress P150H, Vogt Labormaschinen GmbH) is used for the pressing process. The samples (7 cm×6 cm) are placed between two heat-conductive pressing plates and the desired pressing pressure p=F is set using a lever. The pressing time is 15 min per specimen. In a combination of pressing (P) and drying (T), various pressing parameters are first varied. Here, drying is carried out in a climatic chamber (23° C., 10%, 24 h). Subsequently, different drying methods are investigated in the combined process at constant pressing parameters (50° C., 10 N/mm²).

With regard to the nonwoven thickness, there is a clearly greater thickness reduction at higher pressing temperature. The pressing pressure, on the other hand, does not show a clear tendency, whereby a thickness reduction is always evident between 5 N/mm² and 20 N/mm², regardless of the temperature (Table 3.8). Both the WG and the WRV decrease with increasing temperature and pressure, resulting in greater hornification. This tendency is particularly evident when considering the samples pressed at 100° C. The results indicate that, especially when the temperature is increased, agglomeration of the fibers is caused by the significant reduction in thickness. The compression of the material reduces the distance between adjacent fibers to the extent that formation of WBB is increasingly possible. In the literature, a spacing of about 0.25 nm to 0.39 nm is given for the formation of WBB. In addition, an indirect drying process is induced by the high temperature, which favours the formation of stable WBB. SEM images also show a clear compression of the fiber structure at higher pressing temperatures. These results are related to the oven drying. Pressing removes water molecules from the fiber network and thus generates free binding sites on the hydroxyl groups of the glucose molecules. With neighboring glucan chains, new WBBs can thus be formed between free hydroxy groups.

TABLE 3.8
Nonwoven thickness native (8.01 ± 0.23 mm), pressed and rehydrated
(n = 24, N = 2); water content, water retention and hornification of rehydrated
samples with respect to pressing temperature and pressure (n = 12, N = 1).

| nonwoven thickness | | | | |
|---|---|---|---|---|
| in mm | | WG | WRY | hornification |
| gepresst | rehydriert | in % | in % | in % |

23° C.

| | | | | | | |
|---|---|---|---|---|---|---|
| 5 | N/mm² | 0.09 ± 0.01 | 0.50 ± 0.04 | 89.2 ± 3.9 | 362.7 ± 82.9 | 65.8 |
| 10 | N/mm² | 0.09 ± 0.01 | 0.40 ± 0.08 | 82.8 ± 4.6 | 300.3 ± 40.9 | 71.7 |
| 20 | N/mm² | 0.09 ± 0.01 | 0.43 ± 0.12 | 80.0 ± 5.3 | 246.1 ± 45.7 | 76.8 |

50° C.

| | | | | | | |
|---|---|---|---|---|---|---|
| 5 | N/mm² | 0.10 ± 0.01 | 0.39 ± 0.04 | 87.3 ± 1.3 | 374.2 ± 44.6 | 64.7 |
| 10 | N/mm² | 0.09 ± 0.01 | 0.41 ± 0.03 | 84.4 ± 3.8 | 291.7 ± 57.3 | 72.5 |
| 20 | N/mm² | 0.07 ± 0.01 | 0.25 ± 0.02 | 78.6 ± 4.3 | 265.1 ± 22.0 | 75.0 |

100° C.

| | | | | | | |
|---|---|---|---|---|---|---|
| 5 | N/mm² | 0.09 ± 0.01 | 0.21 ± 0.03 | 75.6 ± 1.9 | 223.2 ± 20.5 | 78.9 |
| 10 | N/mm² | 0.07 ± 0.01 | 0.18 ± 0.02 | 69.5 ± 1.8 | 159.8 ± 12.1 | 84.9 |
| 20 | N/mm² | 0.07 ± 0.01 | 0.15 ± 0.01 | 69.0 ± 4.0 | 154.8 ± 7.9 | 85.4 |

The analysis of the mechanical properties also confirms this hypothesis. With unchanged fracture force and fracture strain despite varying pressing parameters, significant differences in the force response are found in the range of small strains. In particular, at a high pressing temperature of 100° C., differences appear at a significance level of p<0, 01. This again indicates the dominant role of WBB with respect to the mechanical behavior of the biomaterial at low strains. With a combination of pressing and drying (P→T or T→P), no change in thickness reduction is achieved with regard to the drying methods investigated (Annex A.9). Moreover, this is independent of the time of the pressing process. When considering the mechanical properties, however, a clear dependence on the time of pressing is evident. With an initial pressing process (P→T), a pronounced stiffening of the fabric occurs. The initial modulus is significantly higher than with a drying process alone and the elongation at break shows comparatively low values. In the case of final pressing after drying (T→P), however, there are no significant changes compared to the sole drying process. A change of the pressing parameters in a combined process has no influence on the timing of the pressing process with respect to the thickness reduction and the WG as well as the WRV. A pressing temperature of 100° C. again results in the greatest hornification. The influence of the time of the pressing process is primarily expressed when considering the initial modulus. With initial pressing (P→T), a greater stiffness of the fabric is observed than with initial drying (T→P). In sum, the results indicate that the sequence of drying and pressing methods determines the structural as well as physical properties of the biomaterial. Subsequent processing methods have only a secondary influence on the properties. For example, a final pressing step before rehydration (T→P) results in a more homogeneous material thickness. An initial pressing step (P→T), on the other hand, tends to result in greater stiffness of the biomaterial.

### Purification

Endotoxins are an integral part of the biological fiber composite BNC due to the synthesis of the fibers by gram-negative bacteria. For the use of the biomaterial in terms of a medical implant material, the removal of these endotoxins is essential to avoid a toxic reaction of the body. For this purpose, the endotoxins present in the bacterial cell wall are removed with the aid of acids or bases. A purification period of 72 h is selected so that the maximum limit of 0.5 EU/ml specified by the FDA for an implant material in the cardiovascular field is not exceeded with sufficient probability. Purification takes place after drying (T), pressing (P) or a combination of both processes (T→P). The cleaning solutions are 0.1 M sodium hydroxide solution or 1 M potassium carbonate. The dry specimens (7 cm×11 cm) are placed in 200 ml of the respective cleaning solution and stored for 72 h in a thermomixer at 80° C. and 350 rpm. The cleaning solution is changed after 24 h each time. After the cleaning process, the samples are rinsed in ultrapure water until the pH is neutralized. Finally, all samples are autoclaved at 121° C. for 20 min (Varioklav type 25T, Thermo Fisher Scientific). With regard to the cleaning solutions used, there are no significant differences in thickness reduction and WG. Regarding the WRV, higher values tend to be found for cleaned samples regardless of the cleaning solution used. Swelling of the fibers is described when treated with alkaline solutions. The increased fiber diameter after the cleaning process allows a larger proportion of the water to be retained. Furthermore, effects of the cleaning process on the mechanical properties of the mechanical properties of the BNC. With only marginal differences with respect to the force and initial modulus, the fracture force and elongation at break show significantly lower characteristic values compared to the samples which have not been cleaned. Accordingly, cleaning with alkaline solutions results in lower tensile strength. An explanation is provided by looking at the molecular structure of the BNC. Acids or alkalis cause the glycosidic bonds between individual AGUs to be cleaved. This leads to a reduction in the degree of polymerization and consequently to hydrolytic degradation of the glucan chains. The force transfer in the mechanical tensile test is determined by the length of the adjacent chain segments, since the tensile strength depends on the force transfer of adjacent molecular chains. Therefore, cleaned specimens tend to achieve a lower mechanical force response. Electron micrographs show the successful cleaning of the biomaterial based on the removed bacterial residues. In both cleaning solutions, the fiber structure without residues of bacterial cells is clearly visible. Overall, the mechanical and structural properties of the BNC are not significantly affected by the endotoxin removal, ensuring the integrity of the biomaterial for use as an implant material.

### Rehydration

The last step of the processing is the rehydration in water. Since the material as an implant material comes into contact with blood in the body, the duration until complete rehydration on the one hand and the maximum possible water absorption on the other hand are of interest. According to DIN 53923, the water absorption capacity is the maximum amount of water that can be absorbed by a structure adapted to a normal climate after storage in water. In addition, the water absorption of dry BNC specimens during storage is quantified by the ambient air humidity by a sorption curve. For the determination of the water absorption capacity, circular samples (Ø2 cm) are laser-cut after drying in a climatic chamber at 23° C. and a subsequent pressing step. The dry weight (mt) is determined and the samples are then placed in 100 ml ultrapure water. Rehydration takes place at 37° C. in the thermal oven. After varying residence times (1 s to 21 d), the samples are removed and water adhering to the surface is removed either by filter paper or by centrifugation. When removing water by filter paper, each side of the sample is placed on filter paper (MN 615, Macherey-Nagel) for 10 s and then its wet weight (mn) is determined. Alternatively, for defined removal of excess water, the samples are centrifuged in centrifuge tubes at 2380 rpm and 20° C. for 20 min (Centrifuge 5920R, Eppendorf GmbH) and then weighed. The percentage water uptake results from:

$$water absoprtion = \frac{{m}_{\text{?}} - {m}_{\text{?}}}{{m}_{\text{?}}} \qquad ( 3.1 )$$

$$\text{?} \text{indicates text missing or illegible when filed}$$

In order to assess the water uptake during storage or further processing, a sorption curve is also drawn up. For this purpose, circular samples (Ø 8 mm) are processed analogously to the water absorption capacity and subsequently stored for 24 h at a relative humidity of 10% and a temperature of 23° C. in a climatic chamber. Starting from this initial value, the rel. humidity is successively increased in 10% steps up to 90%. The respective ambient condition is kept constant for 24 h and then ten samples are taken. The water content of these samples is determined by weighing, based on the dry weight at 10% relative humidity.

The water uptake after different residence times already shows saturation after six hours of storage, which remains unchanged after three weeks. The rapid water uptake within the first five minutes of rehydration is also clearly visible. Moreover, this tendency occurs regardless of the method chosen to remove the excess water. The measurement shows that a rehydration time of about 6 h is sufficient to achieve complete water uptake.

The analysis of the sorption curve shows a steadily increasing tendency of water absorption with increasing relative humidity. However, it is striking that even at 90% rel. humidity the water absorption is less than 20%. The dry BNC is therefore only slightly capable of absorbing moisture from the ambient air. This ensures contamination-free storage of the biomaterial in a dry state at room temperature and represents an outstanding indicator for an application as implant material.

The properties of native and dried-rehydrated BNC were further elucidated. Electron microscopic analysis of native cellulose nonwovens was used to narrow down the diameter of the cellulose fibers of the bacterial strain K. hansenii to 30 nm to 60 nm. The density of the BNC was experimentally determined to be 1.3 g/cm³. Both characteristic values are independent of the selected cultivation period and the ratio of bacteria to nutrient medium during incubation. Only the fiber volume increases with longer nonwoven synthesis.

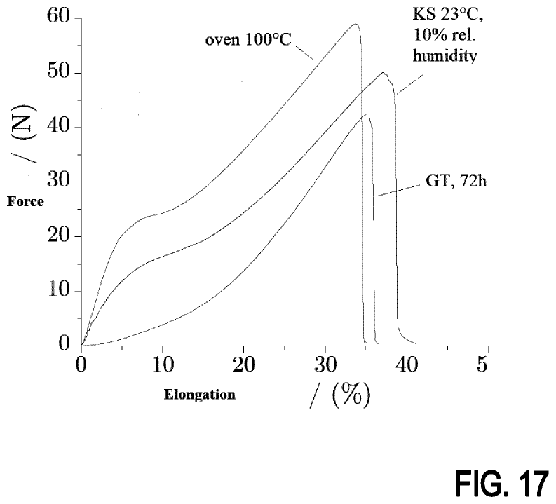

By different variants of post-treatment or processing, a standard process for the production of the biomaterial for an application in the cardiovascular implant field was established (see FIG. 17) and its reproducibility was tested.

A cultivation time of 7 d due to the mechanical strength and a gentle drying in a climatic cabinet at 23° C. proved to be advantageous. Optionally, a pressing process can be performed after drying to improve thickness homogeneity. The cell residues are subsequently removed by cleaning with sodium hydroxide solution. Since cleaning has only a minor effect on the characteristics of the BNC, rehydration in ultrapure water is carried out as standard in the following chapters, unless otherwise mentioned. Here, a rehydration time of 6 h already achieved saturation with respect to the percentage water absorption.

Overall, this type of processing results in an extraordinarily homogeneous, reproducible material structure due to the low standard deviation of the characteristic values, which can potentially be used as an implant material in the cardiovascular field.

### Uniaxial Tensile Loading

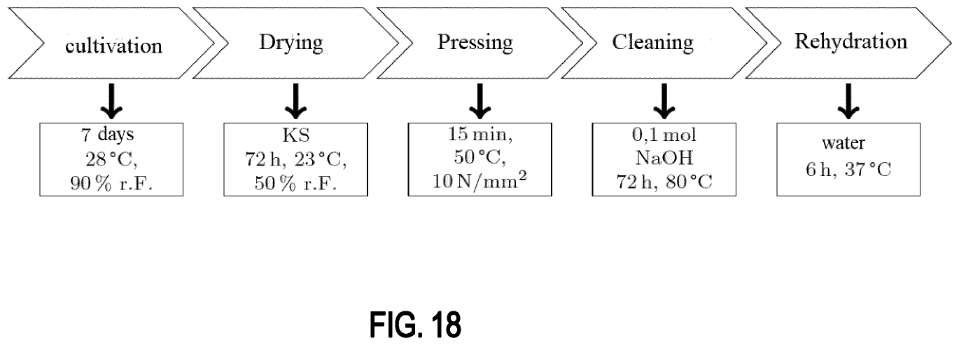

The basis for an evaluation of the deformation behavior in a uniaxial tensile test is the force-elongation curve. The curve of a force-elongation diagram of a specimen that was manufactured according to the process of FIG. 18 can be divided into four sections (A-D). In section A, the load increase starts under continuously linearly increasing force (initial modulus or F-modulus 5%) until a plateau region begins in section B, which characterizes the necking process and thus a rearrangement process of the fibers. The force increase in this area is significantly lower than at the beginning of the load absorption. In section C, a linear increase in force (F-modulus) again follows, since there is presumably a continuous load absorption of the fibers, which are now parallel to the tensile direction, until failure of the specimen finally occurs in section D. Immediately before sample failure, the constriction is (78.1±1.7) % (n=13) with respect to the initial sample width of 5 mm. As discussed in Chapter 3, the mechanical properties of the biomaterial are highly dependent on the cultivation parameters and the post-processing methods chosen. The rehydrated BNC processed shows a fracture stress of >30 MPa as well as an elongation at break of about 40%. This makes the BNC biological material suitable for use in cardiovascular applications, where the mechanical stress is in the range of 1 MPa.

Microscopic observation of the specimen after tensile loading shows failure in several layers. In addition, the arrangement of the fibers at the surface parallel to the loading direction is clearly visible. However, in deeper layers, fiber bundles show isotropic orientation. Furthermore, an increase in specimen thickness can be observed.

### Influence of Humidity and Specimen Geometry

The water content in the fiber network and the number of load-bearing fibers have a significant influence on the mechanical behavior of the hydrogel. Therefore, the effects of different rel. humidity of the BNC specimens and specimen geometry on the force response of the material in the uniaxial tensile test are specifically investigated in the following. Different specimen widths are used to systematically analyse the effect of varying the number of load-bearing fibers on the mechanical strength of the fiber composite.

For the analysis of the influence of humidity and geometry on the mechanical behavior of the biomaterial, uniaxial tensile tests are performed according to the method described in section “Mechanical characterization”. To assess the influence of humidity, the specimens are not measured in ultrapure water but in air. These are native samples with a water content of about 98%, dry (20% RH) and dried-rehydrated samples (70% RH).

Starting from the standard geometry with an inner width of 5 mm, the geometry is then scaled to thinner inner widths (0.3 mm, 0.5 mm, 0.75 mm, 1 mm, and 2 mm) by adjusting the elliptical recess. For specimens wider than the clamping area (10 mm), rectangles with a width of 25 mm or 45 mm are used. The specimen length is always 50 mm and the measurement is performed exclusively in the dried-rehydrated state. In addition to the mechanical properties of breaking force, breaking strain and F-modulus, the work applied to deform the specimens is given below, which is determined by the integral of the force-displacement curve.

The different specimen conditions result in significantly different force-elongation diagrams. In native specimens, about 7 mm to 8 mm thick, almost no force absorption takes place up to an elongation of 10%. Due to the high water content of the porous structure, the fibers are displaced relative to each other at the beginning of load absorption. Only after rearrangement does the applied load lead to longitudinal strain and increasing force response until failure of the specimen. In dry specimens, almost no rearrangement of the fibers at the micro level is possible due to the induced hornification, which is evident from the low elongation at break and the almost linear force increase.

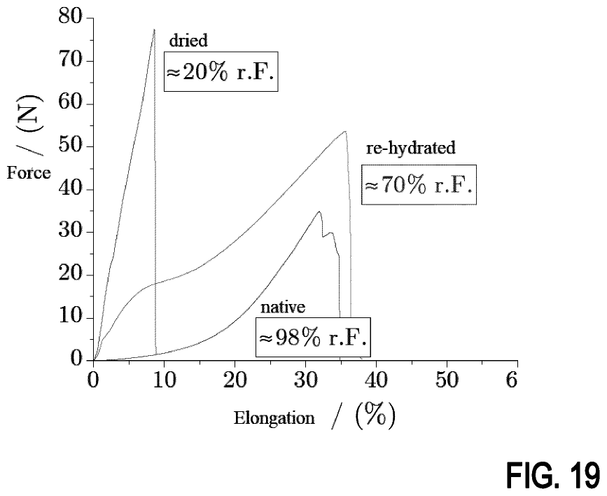

The aggregation of the fibers and additionally formed WBB due to drying lead to a significantly higher breaking strength and a larger F-modulus (F-Modul) than in native or rehydrated samples (Tab. 4.1). The latter show the characteristic course from FIG. 19 with about 70% RH. At the same time, a larger water content allows a pronounced displacement or rearrangement of the fibers relative to each other, which is manifested by a higher elongation at break in native or rehydrated samples. The water content and thus the density of the fiber network thus significantly determine the force response in the uniaxial tensile test.

TABLE 4.1
Mechanical properties and density of BNC specimens with different
relative humidity(n = 25; N = 2).

| | density | breaking | elongation at | F-Modul | Work |
|---|---|---|---|---|---|
| sample | in mg/cm³ | force in N | break in % | in N | in mJ |
| nativ | 14.7 ± 1.1 | 31.7 ± 7.7 | 33.9 ± 7.9 | 191.0 ± 36.8 | 164.7 ± 30.7 |
| trocken | 1177.2 ± 36.1 | 65.8 ± 12.5 | 6.9 ± 1.2 | 916.8 ± 58.6 | 74.8 ± 20.5 |
| rehyd. | 543.2 ± 31.7 | 50.2 ± 3.4 | 41.7 ± 4.5 | 140.5 ± 25.5 | 329.3 ± 37.5 |

The observation of the force-elongation curves of different geometries immediately shows an increase of the breaking force with increasing specimen width. This results from the increasing number of load-bearing fibers with larger specimen geometries. The total work also illustrates the increase in work to be expended for deformation of the specimens. The plateau region in the force-elongation curve, which characterizes the rearrangement process of the fibers, starts with increasing specimen width only at a higher load. Since there are more junctions in the fiber network as the number of load-bearing fibers increases, more force is required to allow initial displacement of the fibers relative to each other. The unfolding of the compressed layered or fiber structure is also evident in SEM images, such as after uniaxial loading of a 45 mm wide specimen. The cross-section outside the loading area show a layered structure in the growth direction of the BNC nonwoven as well as a compact, gapless surface. In contrast the cross-section loaded by the tensile test reveals a pore-like structure, which demonstrates the unfolding of the layer structure and the breaking of fibers and their interconnections by the applied load. This aspect is further manifested by an increase in thickness during the tensile test.

### Auxetic Behavior

Materials usually become thinner under tensile loading because they stretch parallel to the direction of tension. However, there are also cases where the cross-section becomes larger under tensile loading. These materials are called ‘auxetic’ and are characterized by a negative Poisson's ratio v. They occur mainly in porous and composite materials, which allow a change in volume. For isotropic materials the Poisson's ratio is between −1<v<0, 5, whereas for anisotropic materials this value is not limited. Basically, depending on the anisotropy, high positive or negative values are achievable. Examples are synthetic materials such as foams, ceramics, composites or microporous polymers. The auxetic effect has also been demonstrated in organic materials. Negative Poisson ratios also occur in certain directions of fibrous composites. In polymers, such as PTFE, the special microstructure of a nodal-fibril network provides the auxetic effect. All auxetic materials are characterized by non-affine deformations, such as unfolding or unrolling. Regarding cellulose, Verma et al. (2013) reported the auxetic behavior of paper and Tanpichai et al. (2012) reported negative Poisson ratios of bacterial nanocellulose. In the following, the thickness of the BNC during the tensile test is determined experimentally and the Poisson ratio for the material used in this work is derived from it. To determine the thickness of the specimens during the tensile test, an analog laser sensor (IL-S065, Keyence Corporation) is mounted perpendicular to the specimen surface on the test stand (see Mechanical characterization). The recording of the thickness change is performed simultaneously with the uniaxial tensile test. From the recorded force-elongation curve and the data of the laser sensor, the Poisson's ratio v is determined according to equation 4.1.

$$v = - \frac{Δ d}{d} / \frac{Δ l}{l} \qquad ( 4.1 )$$

Here d denotes the initial thickness, Δd the change in thickness, and I the initial length and Δl the change in length of the specimen during the tensile test. Native, dried (climatic chamber) and rehydrated BNC was analysed. Depending on the different specimen conditions native, dry and rehydrated, different developments of the thickness during uniaxial loading are observed. In native samples, which are characterized by a very high water content of more than 98%, the water is squeezed out of the porous hydrogel structure during load application, resulting in a continuous thinning of the sample. Relative to the initial state, the thickness decreases by about 70% until failure of the specimen (Table 4.3). The thickness decrease enters equation 4.1 with a negative sign, resulting in a positive Poisson's ratio close to the value 1. This corresponds to a decrease in volume, which occurs mainly in porous and anisotropic materials and can be explained by the escape of water. Dry BNC samples show the largest thickness increase of about 730%. Due to the reduction in water content and the resulting induced hornification during drying, additional WBBs are formed between the fibers at the free hydroxyl groups, which are considered as fixed network points. Verma et al. (2013) constituted for paper that these network points formed by WBB at fiber junctions are crucial for auxetic behavior. When paper is stretched, the flexible cellulose fibers pull at these network points, thereby displacing adjacent fibers perpendicular to the direction of pull. In dry, compressed fibers, this effect is very pronounced and consequently leads to an increase in thickness, because sliding of the fibers relative to each other is not possible.

TABLE 4.3
Characterization of thickness increase: initial thickness and final
thickness as well as percent change and calculated Poisson's
ratio of native, dry and rehydrated BNC specimens just before
the failure point under uniaxial tensile loading (n = 6, N = 1).

| | initial | final | | |
|---|---|---|---|---|
| | thickness | thickness | change | Poisson's |
| BNC sample | d₀ in mm | d_{f} in mm | in % | ration ν |
| native | 6.35 ± 0.14 | 4.30 ± 0.67 | 67.6 ± 9.2 | 0.95 ± 0.35 |
| (ϵ = 25%) | | | | |
| dried | 0.11 ± 0.01 | 0.79 ± 0.12 | 727.2 ± 77.2 | −102.5 ± 18.3 |
| (ϵ = 6%) | | | | |
| rehydrated | 0.16 ± 0.01 | 1.12 ± 0.08 | 691.3 ± 59.2 | −23.7 ± 2.4 |
| (ϵ = 35%) | | | | |

Also in the case of rehydrated BNC specimens, which have formed irreversible WBB during drying, the effect can be seen in the same way. Here, moreover, the rearrangement process in the plateau region of the force-elongation curve due to the incorporation of water during rehydration is confirmed by a significant, brief increase in thickness. The observation of the negative Poisson's ratio as a function of different strain states for rehydrated specimens also shows the increased expression of the thickness increase at low strains up to 10% due to the reorientation of the fibers at the beginning of uniaxial tensile loading. At larger deformation up to specimen failure, a smaller thickness increase takes place. Thus, overall, auxetic material behavior results for dry and rehydrated BNC due to the compressed, porous fiber network and their anisotropic material structure under uniaxial tensile loading.

### Viscoelastic Behavior

Viscoelastic behavior is characterized by both viscous and elastic deformation of a material. The BNC has amorphous and crystalline regions as well as a large number of hydroxy groups connected by WBB. The BNC hydrogel consisting of BNC fibers and water is experimentally analysed in the following with respect to its viscoelastic behavior. Since the biomaterial BNC is intended to be used in complex loading conditions of the human body environment, the viscoelastic properties as well as the time-dependent behavior are of crucial importance. Multi-cycle tensile tests, relaxation measurements and tensile tests as a function of temperature and strain rate are performed for the characterization. Multi-cycle tensile tests up to a constant force or stress limit differ significantly depending on the force up to which the specimen is loaded. At a force limit of 3 N, a linear viscoelastic behavior is shown. However, when the force limit is increased to 8 N, a partial plastic deformation can be seen. The same behavior is shown for cyclic loading up to a constant yield strength. From a cyclic load up to 5% elongation, less force is required with increasing number of cycles to pull the specimen to the corresponding elongation. At a force limit of 3 N, a continuous decrease of the absorbed energy per cycle is evident. In contrast, at higher force limits, a sudden reduction of 60% and 90% in absorbed energy is evident between the first and second loading cycles. Most of the energy is absorbed in the first cycle. This confirms the observation of the absorbed energy per cycle under cyclic loading with constant proof stresses. It is also noticeable that for cyclic loading to 8 N, almost no reduction in absorbed energy occurs between the second and tenth cycle. This can be attributed to the micro-level fiber rearrangement process prevalent in this strain range. One explanation for the behavior that occurs is the reorientation or reorganization of the fibers along the loading direction due to the applied load. Fiber-to-fiber displacements lead to irreversible absorption of energy. The failure of cross-links at the micro level, which does not lead to macroscopic cracking, ultimately ensures the release of energy and leads to plastic deformation accompanied by an overall high ductility of the biomaterial. The influence of the failure of fiber bonds and hydrogen bonds on the micro level can also be seen when considering the cyclic loading with steadily increasing force limit. The force is iteratively increased by 2 N each time until failure of the specimen occurs. Compared to the reference without any preload, the total energy required to destroy the specimen under cyclic loading with increasing force limit is on average 100 mJ lower. The energy required to tear the specimen is thus significantly reduced when the elastic component not contributing to failure is taken into account. At higher constant force limits, the absorbed energy increases steadily due to the cyclic loading, with the energy decreasing accordingly until the final failure of the specimen. The total energy, i.e. the sum of cyclic preloading and failure measurement, only shows a significant reduction from a force of 8 N compared to the reference without cyclic loading. The reference measurement and the measurement with a cyclic preloading of the specimen to a force of 3 N result in the identical total energy. Accordingly, low forces up to 3 N do not lead to any significant displacement of fibers relative to each other and the associated, microscopic failure of fiber joints. These findings ensure the use of BNC as a cardiovascular implant material, since no mechanical stress in the ductile range (>8 N or 5 MPa) is exerted on it in the human body. According to Zioupos et al. (1994), for example, the load on a heart valve prosthesis is below 1 MPa.

Also relaxation measurements confirm the viscoelastic character of the biomaterial BNC. Relaxation measurements represent a method for characterizing the fatigue or recovery of viscoelastic materials by recording the decrease in the restoring force of a specimen at constant strain as a function of time. The material is stretched by a defined amount and this stretch is kept constant for a certain time. The temporal course of the decrease of the initial tensile force is recorded.

### Dynamic Fatigue Tensile Loading

In vivo, biomaterials are not only statically stressed, but primarily subjected to cyclic as well as time-varying loads. This leads to fatigue phenomena of the material structure, which depend on the type of dynamic loading as well as the repetition rate. The repetition of the same or similar load results in a significantly lower strength compared to static loading. The fatigue behavior of an implant material must therefore be studied in terms of the number of loading cycles and the prevailing loads in the subsequent biological application environment. Therefore, in the following, the biomaterial BNC is analysed with respect to its fatigue behavior under dynamic continuous tensile loading. For the analysis of the fatigue behavior, the experimental setup for dynamic alternating loading is used. The motion is driven by a high performance linear motor (type PS01-23x160H-HP-R, LinMot) with a linear guide (type H01-23x166/180-GF, LinMot). For the of the specimens is a lever with a ratio of 10:1 from motor to tissue. of 10:1 from motor to fabric. The holders for clamping the specimens are attached to a movable aluminium plate, which are connected to the lever by guided linear shafts. The eight fixtures are each connected to a load cell (type U9C, HBM) and are printed using an SLA printer (Form3, Formlabs) made of resin (Grey Pro Resin, Formlabs). The transmission of the sensor data of the load cells is ensured by a universal amplifier (QuantumX MX840B. HBM). In order to stretch the samples to a defined preload, the linear shaft is equipped with a positioning unit. Before starting the measurement, a preload of 1.5 N is set manually. To prevent the tissue samples from drying out during the test, they are placed in a basin filled with ultrapure water. With the help of a software the sensor data are processed and the motor is controlled. In addition, the parameters strain, frequency and repetition rate or duration of the measurement are determined with the software. The material specimens are prepared according to the standard process, their fatigue behavior is investigated at different parameters (strain, frequency and number of cycles) and finally loaded to failure under uni-axial tensile loading. One specimen of each test series is analysed in the SEM with respect to structural changes of the fiber network caused by the dynamic alternating load. Table 4.7 lists the corresponding mechanical properties. Here, F0 denotes the maximum force of the first loading cycle, F_{eq} the saturation force at the end of the measurement period, and the percentage drop denotes the reduction of the maximum force F₀ to the saturation force F_{eq}. In addition, the characteristic values of breaking force and work determined from the uni-axial tensile test are listed. The uniaxial tensile test to failure of the specimen is performed after the fatigue tensile loading in each case. The measurements of different strains (3%, 6% and 10%) are performed for a duration of 1 d at a frequency of 5 Hz. The qualitative curve shows almost no differences. However, the obtained characteristic values indicate a higher percentage decrease in force at a larger cyclic strain. Similarly, the breaking force and work are reduced in the tensile test carried out subsequently. As expected, greater cyclic strain results in greater structural damage to the load-bearing fibers. When viewing the SEM images, this structural change is manifested by fiber strands aligned parallel to the loading direction. Continuous tensile loading at different frequencies (1 Hz, 5 Hz and 7 Hz) and constant elongation of 3% for a duration of 1 d yields no significant differences. The speed at which the cyclic load is applied therefore has no influence on the force response of the material. The length of the measurement or the number of cyclic repetitions (450000, 2000000 and 5000000) results in a lower force response with increasing number of cycles. The analysis of the mechanical characteristics, analogous to the change in strain amplitude, shows a larger percentage decrease in force with longer loading duration. Likewise, the breaking force and the work in the subsequently performed tensile test decrease (Table 4.7). Consequently, with longer load duration, a steadily continuing structural damage of the material occurs.

TABLE 4.7
Mechanical properties of the cyclic fatigue tensile test (F0, Feq and percentage decrease
in force response) as a function of strain, frequency and number of cycles as well
as of the subsequently performed uniaxial tensile test (breaking force, work).

| | | | | |
|---|---|---|---|---|
| F₀ | F_{eq} | percentage drop | breaking force | work |
| in N | in N | in % | in N | in mJ |

Elongation

| | | | | | |
|---|---|---|---|---|---|
| 3% | 12.5 ± 1.1 | 5.4 ± 0.3 | 56.4 ± 3.0 | 47.5 ± 3.2 | 253.2 ± 29.3 |
| 6% | 17.9 ± 1.6 | 5.9 ± 0.3 | 66.9 ± 2.5 | 44.2 ± 6.0 | 209.0 ± 49.2 |
| 10% | 23.8 ± 1.7 | 6.7 ± 0.3 | 71.0 ± 1.4 | 42.8 ± 3.9 | 172.2 ± 26.8 |

Frequency

| | | | | | |
|---|---|---|---|---|---|
| 1 Hz | 10.4 ± 1.6 | 4.5 ± 0.8 | 57.0 ± 4.3 | 41.2 ± 2.9 | 191.6 ± 26.6 |
| 2 Hz | 10.7 ± 1.1 | 4.3 ± 0.5 | 59.1 ± 3.7 | 44.0 ± 3.3 | 225.1 ± 27.6 |
| 5 Hz | 12.5 ± 1.1 | 5.4 ± 0.3 | 56.4 ± 3.0 | 46.7 ± 3.3 | 248.1 ± 30.6 |
| 7 Hz | 12.8 ± 1.0 | 5.4 ± 0.2 | 57.6 ± 3.2 | 43.4 ± 3.7 | 214.7 ± 38.9 |

Cyles

| | | | | | |
|---|---|---|---|---|---|
| 450000 | 12.5 ± 1.1 | 5.4 ± 0.3 | 56.4 ± 3.0 | 47.5 ± 3.3 | 253.2 ± 30.6 |
| 2 Mio. | 12.2 ± 1.3 | 4.0 ± 0.6 | 66.8 ± 5.5 | 39.3 ± 4.6 | 153.8 ± 30.7 |
| 2 Mio. | 2.9 ± 0.4 | 1.8 ± 0.3 | 36.7 ± 5.6 | 8.3 ± 5.3 | 29.4 ± 27.3 |
| (Perikard) | | | | | |
| 5 Mio. | 17.5 ± 1.3 | 4.9 ± 0.3 | 72.2 ± 1.6 | 32.6 ± 4.4 | 128.9 ± 17.1 |

In comparison to porcine pericardium, which is an established material for implant applications, a simultaneous force progression is shown at a load up to 2 million cycles. Porcine pericardium, consisting of collagen and elastin, is characterized by a lower percentage decrease in force, since higher restoring forces of the viscoelastic material prevail under cyclic loading due to the elastic portion of elastin (Table 4.7). The comparison suggests that the mechanical integrity of the BNC for an application as an implant material is ensured despite the reduced force response at long loading duration.

### Fracture Mechanics

For the crack propagation (Load perpendicular to crack propagation) of plant cellulose, only a fraction of the work to be done by BNC is required. This is probably due to the manufacturing method of plant cellulose and its associated lower formation of interfibrillar bonds. The fracture force, which defines the onset of crack propagation in this particular case, is about 20 N for all specimens. Observation of the microscopic SEM images clearly shows the difference between the loaded and non-mechanically stressed region of the specimen. The deconvolution of the compressed layer structure due to the applied load of the uniaxial tensile test can be seen by the increase in thickness of the specimen. There a clearly defined transition to the unloaded region is evident. The critical stress intensity factor of BNC according to Eq. 4.7 gives a value of 10 MPa to 13 MPa √m (Y_{I}=2.83) for the specimens with a notch on one side (5 mm). Compared to other biological materials, such as wood (KIC=10 MPa√m), bone (KIC=4 MPa√m) or cartilage (KIC=0.08 MPa√m), the fracture toughness determined for the BNC ensures an equivalent or, in some cases, even greater resistance to crack propagation. The stress intensity factor for mode I (crack propagation perpendicular to the crack surface) is described by equation 4.7, where σ represents the nominal stress, a the crack length and Y_{I} a geometry factor.

$${K}_{I} = σ \cdot \sqrt{π a} \cdot {Y}_{I} \qquad ( 4.7 )$$

Also the transverse shear, i.e. the displacement of the crack surfaces transverse to the crack direction, is examined in addition to the perpendicular crack propagation. The resistance of the incision to further tearing is determined. In the following, a so-called leg tear propagation test according to DIN EN ISO 13937-2:2000-06 is carried out to analyse the biomaterial BNC with regard to this type of loading. Specimens with dimensions of 38 mm×20 mm are used, which have an indentation with a length of 18 mm. The legs of the specimen are clamped in special 3D-printed holders (Ultimaker 3, Ultimaker) without preload. The tear propagation force F_{W} is determined along the tear propagation distance l_{W}. This is always 20 mm due to the specimen geometry. The tear propagation force FW is the tensile force required to tear the indentation further. The tear propagation force F_{W} of plant is approximately 0.5 N. For rehydrated BNC there is a higher tear force and total energy to tear the samples than in the case of plant cellulose. In addition, some layer detachment occurs in rehydrated BNC samples, resulting in a 30% higher tear propagation force than in plant cellulose. Since the fibers are isotropically distributed in all directions in the case of BNC specimens at the nano level, fiber connections perpendicular to the onward travel distance influence the material behavior in neighboring areas. The consequence is the separation of a cellulose layer, instead of a straight-line further travel path. SEM images show the qualitatively different behavior between straight-line crack propagation and layer separation. In the case of crack propagation, a repositioning and bundeling of fiber strands is observed in the direction of the tensile force. The influence of the force is locally limited and bundles, which were originally oriented perpendicular to the crack propagation, are oriented parallel to it. When delaminating, fiber bonds become loosened between the successively formed growth layers during mat synthesis, which is evident by a loose fiber network structure. In the area not loaded by the tensile force a compact surface topography is evident, which does not contain individual loose fibers. Overall, the structure of the BNC thus exhibits anisotropic material behavior. Fibers within one plane, i.e. perpendicular to the growth direction of the nonwoven, result a very homogeneous crack propagation behavior. Parallel to the growth direction, however, there are structural weak points between the successively formed layers, which are characterized by the detachment of the material from the layers and occur in about 60% of the tear propagation tests carried out.

### Puncturing Behavior

Puncture strength tests are used to determine the puncture or failure properties of a material and thus its strength against point loads. A puncturing element is moved perpendicular to the specimen surface at a constant speed centrally on the specimen until failure occurs. In the following, the test method developed for polymer films according to DINISO 7765-2:2009-02 is modified and the puncture behavior of the biomaterial BNC is characterized using various test specimens. The test specimens are measuring probes with a spherical head of varying diameters (0.5 mm, 1 mm and 4 mm). In addition, a surgical needle (Ø 40 μm to 280 μm) is used to characterize the piercing behavior in an analogous way. The BNC in the dry state shows a similar behavior to that of plant cellulose, but with a higher damaging force. This can be attributed to the more stable, fiber-reinforced structure of BNC. In rehydrated BNC specimens, the incorporated water has a significant effect on damage deformation. The water favors larger forces and displacement of the fibers relative to each other, which also leads to a significantly smaller increase in the force profile in the linear region of the force-displacement curve, i.e., lower stiffness, compared to dry BNC. The elongation of the BNC is thereby possible to a greater extent, so that irreversible structural changes are induced only after greater deformation. The total energy required to induce damage to the BNC specimen is thus much higher than for plant cellulose. Towards puncturing BNC is extremely stable and exhibits outstanding strength and resistance. The internal microstructure, characterized by an isotropic distribution of the fibers at the nanoscale, favors the integrity of the fiber network under point loading. This can be seen when looking at the puncturing behavior with a surgical needle with BNC compared to aluminum foil. The aluminum foil has an order of magnitude lower total energy and damage force. After the needle has been inserted in the aluminum foil, the force drops immediately and steadily. The rehydrated BNC, on the other hand, exhibits an almost constant after the initial insertion. The internal microstructure of a fiber-reinforced material in the area of punctual damage thus counteracts the propagation of the induced failure.

### Cut Resistance

For a cardiovascular implant application of the BNC in combination with a metal stent, the implants are concentrically crimped, for example, for placement in the catheter as part of a minimally invasive implantation. The high cut resistance of the BNC compared to the stent struts is advantageous. In order to characterize this cut resistance, the average behavior is investigated. The test specimen is a blade with a length of 9 mm, which strikes the specimen centrically at an angle of 30°. For the further cutting behavior at a defined preload of the specimen, the test specimen (razor blade, length 35 mm) is fixed in a fixture and moved in the direction of the specimen clamped perpendicular to it. For this purpose, a biaxial setup is used, which allows simultaneous clamping in x- and y-direction. The rehydrated BNC specimen, which is clamped in the y-direction, is pre-stretched in a defined manner (elongation 10%, 20%, 30%) before the measurement. The blade is then moved in the direction of the specimen clamped perpendicular to it. Dry BNC exhibits a similar behavior in average tests as vegetable cellulose, whereby the total energy for the average denier is higher than for vegetable cellulose. This represents the high strength of the compact fiber network of the BNC. At about the same damage force, this method also shows the influence of water in rehydrated BNC specimens towards more flexible material behavior. This is indicated by twice as large a damage path. The results of the re-cutting tests show with increasing pre-stretching the total amount of work required to cut the specimen completely. The incision of the specimen by the blade is induced more rapidly with greater pre-strain. This is evidenced by a smaller damage path and a steeper increase in force at the beginning of the measurement. The internal stress in the sample at greater pre-strain also ensures that less force needs to be applied for initial damage. Compared to plant cellulose and dry BNC, the rehydrated BNC exhibits the highest cut resistance.

### Bending Strength or Dimensional Stability

Bending stiffness was compared to porcine pericardium as an implant material for biological prosthetic heart valves. Compared to porcine pericardium, the BNC is much more resistant to bending and thus allows the generation of dimensionally stable structures. In order to analyze the bending strength and dimensional stability of the BNC in dependence of the different process variants, native (synthesis time 7 d), dried BNC (climate chamber, KS) and dried-pressed, rehydrated BNC samples are investigated. High temperatures (100° C.) during drying or pressing indicate the increased formation of hydrogen bonds (hornification). The additional bonds in combination with a stronger compression of the fiber network, i.e. a lower thickness and higher density, lead to the stiffening of the material. This was shown uniaxial tensile loading of the material by a higher initial modulus. Analogously, the drop capacity of these specimens tends to yield a higher drop coefficient in the context of the flexural strength investigations.

### Example 9

The embedding of hygroscopic exchange materials is described as follows:

The functional groups of the BNC allow the physical properties to be changed by the formation of hydrogen bonds with other substances. These exchange materials, also referred to below as stabilizers or plasticizers ensure specific prevention of hornification during drying by bonding to the free hydroxyl groups of the BNC. As already explained above the drying process of the BNC the collapse of the three-dimensional fiber structure and the associated agglomeration of the fibers leads to the increased formation of interfibrillar hydrogen bonds, which restrict the relative movement of the BNC fibers and thus lead to a stiffer and less flexible material behavior. By introducing stabilizers before drying, intermolecular forces are reduced and the flexibility of the material is improved. The stabilizers not only coat the surface of the material, but also penetrate into the open-pored BNC matrix. This makes it possible to influence the mechanical force response of the BNC. Glycerin and polyethylene glycol (PEG) have proven to be effective stabilizers. Both substances are characterized by their hygroscopicity, low toxicity values and outstanding water solubility. A wide range of applications results from their ability to absorb and bind moisture from the environment, so that both substances are already used as moisturizers and softeners in the cosmetics and textile industries.

Glycerol or Glycerin (IUPAC name: propane-1,2,3-triol) has one primary and two secondary hydroxyl groups, and is from a chemical point of view, a trivalent alcohol (molecular formula: C₃H₅(OH)₃). From a physical point of view glycerol is a clear, viscous liquid which, due to the hydroxyl groups, has a similar groups, it has a solubility comparable to water and simple aliphatic alcohols.

PEG (IUPAC name: polyoxyethylene) is a synthetic polymer that is suitable for use in implant applications due to its hydrophilic and biocompatible properties. Chemically, PEG has two terminal hydroxy groups with the general molecular formula H(OCH₂CH₂)ₙOH. Up to a molar mass of 600 g/mol, PEG is a non-volatile, hygroscopic liquid at room temperature. The outstanding water solubility of PEG is due to the distance between the repeating oxygen atoms in the polymer chain. This corresponds approximately to the spacing of oxygen atoms in which allows the formation of a water stub network between the polymer chain and the water molecules.

By immersing or rinsing the native BNC hydrogel in a stabilizer solution of defined concentration, the glycerol or PEG molecules diffuse through the pore structure into the BNC fiber matrix. The formation of hydrogen bonds between the stabilizer molecules and the BNC fibers challenges the mobility of the polymer chains. In addition, the introduced molecules act as placeholders during drying and thereby increase the porosity and the spacing of the BNC fibers. Depending on the concentration of the selected stabilizer solution and the different molecular weight and hydroxyl content of the substances, characteristic physical properties result of the stabilized-dried BNC after rehydration.

In the following, the diffusion behavior and the incorporation of the hygroscopic exchange substances into native BNC are analyzed.

### Stabilization Process and Detection of Incorporation

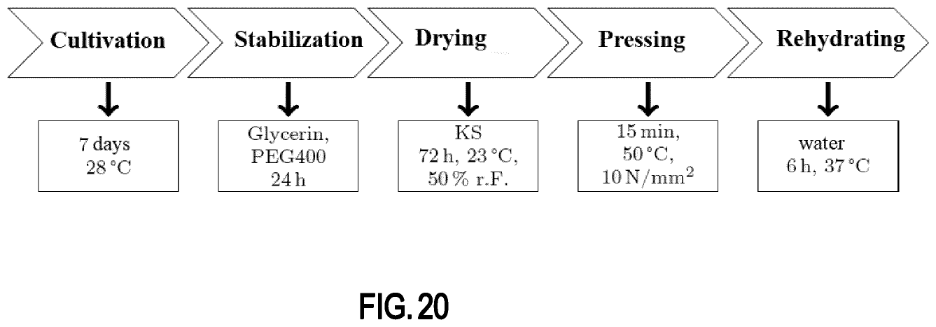

Stabilization of the biomaterial with glycerol and PEG400 is performed after static synthesis in an incubator (7 d, 28° C.) and a subsequent rinsing process with ultrapure water. The inclusion of the stabilizers thus takes place before the drying process, in order to prevent the formation of hydrogen bonds by the removal of water. The subsequent post-treatment is carried out after standard process (see FIG. 20).

Rectangular sample geometries (7 cm×12 cm) are generated and placed in (7 cm×12 cm) are generated and placed in 400 ml stabilization solution. The solution is composed of the respective stabilizer glycerol (anhydrous) or polyethylene glycol 400 (PEG400) and ultrapure water (Milipore Direct-Q5: Merck Chemicals GmbH) with different mass fractions. The BNC samples are rinsed for 24 h on a shaker (Promax 1020, Heidolph Instruments GmbH & Co. KG) at 140 rpm in the respective stabilization solution. They are then processed according to the standard stabilization process (see also FIG. 20). After the stabilization process, the native square BNC samples are first dried in the KS in the respective stabilizer concentration (1 wt % to 50 wt %) and finally placed in a vacuum dryer (Christ Alpha 1-2, Martin Christ Gefriertrocknungsanlagen GmbH) for 24 h. The samples are then dried in the KS. Visual inspection of the samples was performed using a transmitted light plate. It can be clearly seen that the stabilized-dried samples, regardless of the type of stabilizer, show higher transparency compared to the reference sample, which was dried without prior stabilization. The reference sample appears completely opaque, whereas the transparency is already evident at low stabilizer concentrations. In the literature, the refractive index of BNC is given as 1.5 to 1.6. A glycerol-water solution of varying concentration has a refractive index of 1.40 for a 50 wt % solution to 1.30 for a very low concentration glycerol solution. As the glycerol concentration increases, the refractive index of the solution approaches that of the BNC fibers, and therefore the composite appears more transparent. The samples treated with 1 wt % stabilizer solution show incomplete saturation of the material in the center of the sample. There are almost no differences between the concentrations of 5 wt %, 10 wt % and 20 wt %. At a concentration of 50 wt % stabilizer solution, the specimens have the samples have the greatest thickness, which explains the lower transparency due to increased interfacial transitions. Haptically, a concentration above 5 wt % results in a softer and more flexible material than concentrations lower than 1 wt %. The cross-section of the samples also illustrates the extraordinarily homogeneous distribution and incorporation of the stabilizer molecules. Electron micrographs of the surface topography show, comparable to visual inspection, a continuous hydrate shell of the stabilizers from a concentration of 5 wt %. At a lower concentration of 1 wt %, individual fiber strands are clearly visible, which are comparable to the surface topography of the non-stabilized reference sample. The stabilization process for BNC thus probably takes place completely at a concentration of 5 wt %. At a concentration of 10 wt % and above, even when looking at the cross-section of the sample, complete wrapping by the stabilizers is present, so that no individual fibers or fibrils are discernible. This is seen in the same way with both stabilizers (glycerol, PEG400). Overall, the methods show homogeneous incorporation of the substances from a concentration of about 5 wt %, regardless of the choice of stabilizer. The diffusion process of the substances into the BNC fiber network up to saturation of the material was investigated using glycerol and PEG400 solutions with concentrations of 5 wt %, 10 wt % and 20 wt % with the aid of spectroscopic measurements, e.g. Fourier transformed infrared spectroscopy (FTIR)). The time of 95 wt % saturation (saturation time) of both stabilizers as a function of the thickness of the native BNC is shown in Tab. 5.1 and Tab. 5.2.

TABLE 5.1
Saturation times of glycerol as a function of the concentration of the
stabilizing solution and the sample thickness at 95% saturation.

| concentration 5 wt % | | concentration 10 wt % | | concentration 20 wt % | |
|---|---|---|---|---|---|
| thickness | time | thickness | time | thickness | time |
| in mm | in min | in mm | in min | in mm | in min |
| 3.0 | 277.7 | 1.9 | 190.9 | 3.2 | 287.4 |
| 3.8 | 341.4 | 2.8 | 244.2 | 3.5 | 297.6 |
| 3.9 | 301.0 | 3.7 | 293.0 | 3.7 | 306.5 |
| 4.0 | 327.4 | 3.8 | 281.3 | 3.8 | 319.0 |
| 4.1 | 284.3 | 3.9 | 292.4 | 4.0 | 320.7 |
| 4.1 | 306.6 | 4.3 | 291.2 | | |
| 4.6 | 872.6 | 4.7 | 377.3 | | |

TABLE 5.2
Saturation times of PEG400 as a function of the concentration of the
stabilization solution and the sample thickness at 95% saturation.

| concentration 5 wt % | | concentration 10 wt % | | concentration 20 wt % | |
|---|---|---|---|---|---|
| thickness | time | thickness | time | thickness | time |
| in mm | in min | in mm | in min | in mm | in min |
| 2.8 | 517.8 | 2.7 | 493.5 | 3.7 | 525.8 |
| 2.8 | 479.5 | 2.8 | 491.0 | 3.0 | 538.0 |
| 3.1 | 510.6 | 2.9 | 534.8 | 3.1 | 556.5 |
| 3.4 | 552.2 | 3.0 | 503.1 | 3.4 | 619.0 |
| 4.0 | 805.8 | 3.1 | 512.3 | 4.4 | 761.8 |
| 4.3 | 723.0 | 3.2 | 520.9 | | |
| | | 3.5 | 605.5 | | |

There is a clear dependence on longer saturation times for higher thicknesses of the native BNC samples. A higher concentration of the stabilization solution does not result in longer stabilization times. The saturation times for glycerol as a function of sample thickness are in the range of 200-400 min. For PEG400, the saturation generally takes longer than for glycerol (500-700 min). It is clear that the stabilizers show equivalent saturation times at approximately the same sample thickness, but at different concentrations of the stabilizer solution. There is therefore no dependence on the selected concentration of the stabilizer solution. for the same specimen thickness. On the other hand, the observation of the same solution concentrations at different specimen thicknesses indicates significantly longer saturation times with increasing specimen thickness. In general, stabilization with PEG400 results in a longer saturation time than with glycerol, even at the same sample thickness. glycerol, which is due to the larger molecular mass of 400 g/mol for PEG400 compared to compared to 92.09 g/mol for glycerol. Overall, it turns out that the determined saturation times after extrapolation to the average thickness values of native BNC in the of native BNC in the range of 6.5 nm to 7.5 mm shows a stabilization time of 12 h for glycerol and 24 h for PEG400 is sufficient to ensure complete saturation of the material.

### Swelling Behavior

The incorporation of hygroscopic substitutes into native BNC is carried out with the aim of the aim of preventing potential hornification of the material during drying. The intercalated stabilizers, which occupy free hydroxy groups of the fibrils and thus prevent the formation of interfibrillar hydrogen bonds during drying, are partially washed out again during rehydration. This makes it possible for increased water is deposited in the fiber network structure, resulting in an extraordinary swelling capacity of the material.

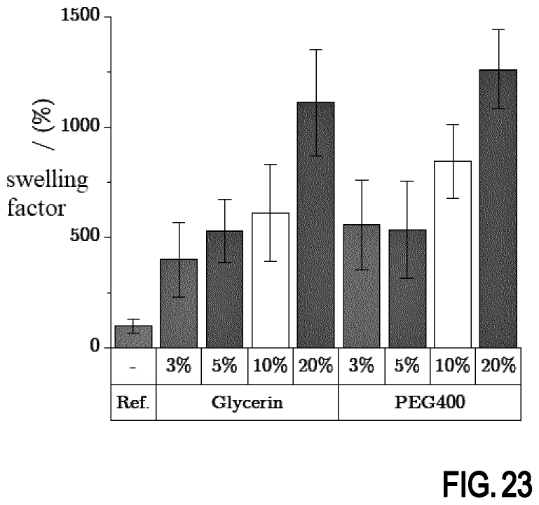

The swelling behavior of stabilized-dried BNC is quantified by tactile thickness measurement. The stabilization is carried out with different concentrations of stabilizers glycerol and PEG400 for 24 h. After drying at ambient conditions for 72 h, the BNC is placed in ultrapure water rehydrated (24 h). Alternatively, before rehydration, a pressing step is carried out. In addition to the absolute thickness values of the samples, the swelling factor (QF) is calculated as the quotient of the rehydrated thickness and the dry or pressed thickness. Furthermore, the water content and water retention capacity are given as indicators for the for the amount of water stored by the rehydration process. The stabilization step, regardless of the concentration of the stabilizing solution and the type of stabilizer, results in a small increase in thickness. Drying leads to a reduction in thickness as a function of the concentration of the stabilizing solution. At high concentrations of concentrations of 100% is only about 10%, while at low concentrations of e.g. 5%, however, up to 95%. These differences in the dry state have a significant effect on the swelling factor, which is lower at higher concentrations of the stabilization solutions. Between the stabilizers Glycerin and PEG400, no differences were observed. In comparison with the reference samples, which were not stabilized, a significantly higher swelling factor was observed even at very low concentrations (see FIG. 23). For many applications in the biomedical field, such as in minimally invasive surgery, a low thickness of the implant material is useful in the dry, storable state. Therefore, after drying, a pressing step is performed in order to minimize the thickness of the material. A subsequent rehydration impressively shows the change of the swelling factor depending on the stabilizer concentration compared to the stabilized-dried BNC without a pressing process. The absolute thickness values in the pressed condition are in the range of less than 0.2 mm regardless of the concentration considered and increase by 400% to 1100% due to rehydration. The increased incorporation of water during rehydration is also reflected in the WG and WRV values, which, like the swelling factor, tend to increase with increasing stabilizer concentration (Table 5.5).

TABLE 5.5
Change in swelling behavior due to a pressing step following drying
to reduce the thickness in the dry state. Shown are the thickness in
native, pressed and rehydrated state as well as the swelling factor
(n = 16; N = 2), the WG (n = 12; N = 1) and the WRV (n = 12; N = 1).

| | native | pressed | rehydrated | QF | WG | WRV |
|---|---|---|---|---|---|---|
| | in mm | in mm | in mm | in 9% | in % | in % |
| Ref. | 7.84 ± 0.48 | 0.15 ± 0.04 | 0.16 ± 0.01 | 97.96 | 67.9 ± 6.5 | 131.4 ± 16.9 |

Glycerin

| | | | | | | |
|---|---|---|---|---|---|---|
| 3% | 8.12 ± 0.41 | 0.15 ± 0.02 | 0.60 ± 0.24 | 400 | 84.0 ± 3.1 | 342.3 ± 67.7 |
| 5% | 7.81 ± 0.32 | 0.17 ± 0.02 | 0.65 ± 0.22 | 529 | 90.5 ± 2.3 | 535.8 ± 72.2 |
| 10% | 8.40 ± 0.43 | 0.18 ± 0.03 | 1.10 ± 0.35 | 611 | 94.1 ± 0.7 | 644.4 ± 89.8 |
| 20% | 6.13 ± 0.28 | 0.18 ± 0.02 | 2.00 ± 0.37 | 1111 | 95.3 ± 0.8 | 653.5 ± 49.0 |

PEG400

| | | | | | | |
|---|---|---|---|---|---|---|
| 3% | 6.67 ± 0.84 | 0.14 ± 0.02 | 0.78 ± 0.26 | 557 | 80.2 ± 9.4 | 458.4 ± 81.5 |
| 5% | 7.85 ± 0.49 | 0.17 ± 0.02 | 0.91 ± 0.36 | 535 | 94.0 ± 1.2 | 680.8 ± 47.1 |
| 10% | 7.29 ± 0.70 | 0.18 ± 0.02 | 1.52 ± 0.25 | 844 | 94.9 ± 0.9 | 722.8 ± 58.7 |
| 20% | 6.13 ± 0.28 | 0.18 ± 0.02 | 2.27 ± 0.20 | 1261 | 96.0 ± 0.9 | 725.7 ± 52.1 |

The choice of stabilizer concentration thus enables the material to swell to a defined thickness through rehydration. This opens up new possibilities in the medical field such as minimally invasive implantation, where, due to the limited catheter diameter, only very small thicknesses of the of the implant material in the dry, storable state are permissible. In addition to the swelling behavior, the incorporation of hygroscopic substitutes into the fiber network of the BNC enables a targeted variation of the mechanical force response in the uniaxial tensile test. In the following section the mechanical properties of the BNCs stabilized at different concentrations of glycerol and PEG400 stabilized-dried BNC after rehydration are analysed. The force strain diagrams are analysed with respect to the breaking force and strain as well as the initial modulus and the total work analysed. The analysis is carried out in stabilizers glycerol and PEG400 as well as the selected concentrations of the stabilizing of the stabilization solutions (3 wt % to 20 wt %). The stabilization of the material is carried out for 24 h before drying. The observation of the force-elongation diagrams impressively shows the change of the force curve at the beginning of the load application for stabilized-dried BNC specimens. Compared with the reference specimens, which were not stabilized, less force is required to deform the specimens at small strains of up to about deformation of the specimens. This manifest itself on the one hand in a significantly lower initial modulus of 20 N to 50N and on the other hand in a generally lower total work required for the failure of the specimens. With increasing concentration of the stabilization solution, these two characteristic values tend to decrease. In turn, both fracture force and strain remain almost unchanged. The results can be explained by the greater thickness of the stabilized-dried specimens after rehydration. In the previous section, the change in thickness due to the stabilization process was explained in the context of the swelling behavior. Due to the leaching of the substances and the resulting increased incorporation of water into the fiber network, a displacement of the fibers relative to each other is made possible without great force. This rearrangement of the fibers is due to the prevented formation of hydrogen bonds between the fibers during drying due to the incorporated stabilizers. Only after the reorientation of the fiber structure, similar load absorption of reference and stabilized-dried specimens up to complete failure occurs from an elongation of about 25%. In the case of the reference specimens, the hornification induced by the drying limits the rearrangement or displacement of the fibers in relation to each other, which leads to a more rigid material behavior at the beginning of the load application. This manifest itself in an 10-fold higher initial modulus compared to stabilized-dried samples. The incorporation of hygroscopic exchange substances into the fiber network of the BNC before drying leads to softer or more flexible material behavior in the range of small loads up to about 10 N. The concept thus represents a modification of the BNC, whereby the force response of the biomaterial under physiological of the biomaterial at physiological loads (<10 N).

### Example 10

In this example, the biomaterial BNC is applied in the context of various cardiovascular implants. In addition to its use as a potential vascular graft substitute the use as a sheath of stent grafts is also realized. With the focus on three-dimensional shaping, a method is being developed for the production of a sutureless tissue component of BNC for percutaneous aortic valve replacement is developed. By combining with locally swellable BNC, a concept for prevention of paravalvular leakage in transcatheter aortic valve implantation (TAVI) is additionally presented.

### Bacterial Nanocellulose as Implant Material for Vascular Prostheses

In vascular surgery, surgical bypass is a potential therapy for conditions such as arterial occlusive disease or aortic aneurysm as a result of arteriosclerosis. Vascular grafts replace, bypass or preserve the function of occluded or diseased blood vessels. The chosen graft is usually taken from the patient (so-called autograft or autologous graft). However, this requires additional surgical This, however, requires additional surgical interventions and is associated with limited availability. Therefore, the use of synthetic vascular grafts made from polyethylene terephthalate (PET/Dacron) or (expanded) polytetrafluoroethylene (ePTFE) has become has been established. However, these are only used clinically for vascular grafting with a diameter >6 mm, since smaller diameters may lead to neointimal hyperplasia or thrombus formation due to hemodynamic disturbances. An alternative for small vessel diameters (<6 mm) should be provided by natural or synthetic polymers.

In the literature, static mostly static synthesis methods in bioreactors are used. The formation of the BNCs takes place vertically on shaping glass cylinders, for example (Klemm et al. (2001), Yamanaka et al. (1990)). However, the length of the graft is limited and a long cultivation time (7 d to 14 d) is required. Alternatively, synthesis in the radial direction on one or concentrically arranged, oxygen-permeable silicone membranes (Hong et al (2015). established (Hong et al (2015), Bäckdahl et al (2011), Bao et al (2021)). Besides the long cultivation time, the low mechanical strength and a heterogeneous layer structure strength as well as a heterogeneous layer structure should be mentioned as disadvantages.

### Bioreactor

In the following, the three-dimensional shaping of the BNC in an in-house bioreactor under dynamic, horizontal rotation of a cylindrical profile. profile. For the three-dimensional synthesis of the BNC, an exemplary bioreactor consisting of three cultivation units was developed with a horizontal rotation of the cultivation surface under a constant oxygen environment. The basis of the bioreactor is a modular platform consisting of aluminium profiles and acrylic plates for positioning the individual units. of the individual units. The drive unit is driven by a geared motor (e.g. 12V, model RB350600-0A101R, Modelcraft with a reduction ratio of 1:600 corresponding to a load speed of 9 rpm). As bearing for FDM print (Ultimaker 3+, Ultimaker) serves as a bearing for the motor on the module platform of the bioreactor. In addition, the motor housing a recess is provided for the use of a Hall sensor (Hall-E_ect Bricklet, Tinkerforge) to detect the speed. A spring coupling (FKZS 1225, ABP Antriebstechnik GmbH) induces a magnetic field during rotation, which is field during rotation, which is detected by the Hall sensor and thus cyclically recorded each revolution of the shaft. The speed is calculated using the data from the Hall sensor with software. The gearbox consisting of shafts, toothed belts and toothed wheels is used to transmit the motor torque to the individual rotation profiles. The motor is connected via connected to the main toothed belt shaft via the spring coupling and transmits the to all three toothed belt shafts by brass spur gears (Ø20 mm). The core of the bioreactor is represented by the rotation profiles and reactor vessels. Both are, if not mentioned otherwise are made of an engineering resin. This is particularly suitable because of its excellent sterilizability. The reactor vessel contains the nutrient medium and the bacterial suspension during cultivation.

### Process for Manufacturing the Vascular Prostheses

The production of the vascular prostheses is divided into cultivation or synthesis in the bioreactor and subsequent post-treatment steps. The cylindrical rotational profile is designed according to the desired diameter. For the attachment of the brass gears, both ends of the profile are fixed to a diameter of 2.8 mm. For synthesis, the reactor vessel (100 mm×38 mm×21 mm, wall thickness 4 mm) is filled with nutrient medium (36 ml) and bacterial suspension (2 ml) so that the rotation profile is in contact with the medium. Due to the rotation with 10 rpm, BNC with a homogeneous thickness is formed on the profile surface. Excess BNC, which is in the reactor vessel, is removed manually after 12 h on a regular basis. The thickness of the BNC tube is largely determined by the duration of the synthesis. To achieve a dimensionally stable BNC tube with the smallest possible wall thickness, a cultivation process has been a cultivation time of 3 d has proven to be effective. After successful cultivation of the BNC tubing, it is rinsed in ultrapure water and then, in order to reduce the thickness, it is placed on the profile in the bioreactor with slow rotation (3 rpm). Optionally, laser cutting is performed in the dry state. After rehydration in ultrapure water for 1 h, the BNC tube is manually removed from the profile, final laser cutting is performed and finally washed in 0.1 mol NaOH for 24 h for endotoxin removal. For final use, the BNC tubing is steam sterilized and stored sterile in 0.5% glutaraldehyde.

### Characterization

The manufactured prototypes for a vascular substitute made of BNC are described in the following appearance, mechanical properties and wall thickness. In addition, a comparison is made to a method of static BNC synthesis on a three-dimensional silicone tubular membrane is drawn.

In an example a vascular substitute made of BNC was mounted on a rotary profile (Ø8 mm) for 72 h at a rotational speed of 10 rpm in the bioreactor. A dimensionally stable tube with an exceptionally homogeneous surface texture is obtained. Another tube which, with otherwise identical parameters was synthesized for 48 h with otherwise identical parameters. This results in a loss of dimensional stability due to a reduced cross-linking of the fiber structure of the fiber structure and reduced wall thickness. The BNC synthesized in a silicone tube shows no dimensional stability even after a synthesis time of 7 d, because in this method the oxygen supply through the silicone membrane is limited. For further assessment, images are taken with a stereomicroscope (SZX10, Zeiss AG) and the scanning electron microscope are used. The extraordinarily homogeneous wall thickness in the axial direction of the of the BNC tubing is evident both in the native state and in the rehydrated state. No irregularities are discernible in the radial direction either. The cross-section also shows a uniformly compacted fiber structure. The synthesis of a BNC vessel set in the bioreactor is thus characterized by outstanding surface quality and homogeneity. In addition to the optical integrity of the BNC vascular prostheses, the wall thickness or the wall thickness of the tubes and their mechanical strength are of decisive importance for their use as vascular prostheses. In the following, at different parameters, BNC tubes are synthesized and their wall thickness is determined in the native (Dnative) and rehydrated (Drehy) states. In the native state, the thickness is measured optically by stereomicroscope images, in the rehydrated state by tactile thickness measurement. For the mechanical analysis, tubular rings with a width of 5 mm are generated using a CO₂ laser and loaded in the radial direction until failure. From the force-strain diagrams, the force to failure (breaking force) and the work required to achieve it. The corresponding data are shown in Table 6.1. An increase in the rotational speed for the same synthesis time leads to a greater native and rehydrated wall thickness of the BNC tubes. This is accompanied by an increase in the breaking force and mechanical work until failure due to the larger number of load-bearing fibers. In general, a rotational speed of 10 rpm or more is preferable, since inhomogeneous growth of the BNC growth of the BNC tubes in the axial direction was observed. The diameter of the rotational profiles has no effect on the wall thickness and the mechanical properties. This allows the BNC tubing to be used regardless of the diameter of the vascular diameter of the vascular graft and ensures a wide range of applications in vascular surgery. In addition to the rotational speed, the duration of synthesis is a decisive factor in determining the mechanical strength of the BNC tubing. With longer synthesis, the native and consequently also the rehydrated wall thickness and the mechanical properties increase. In addition, from a synthesis time of 72 h onwards, an internal mechanical stability of the fiber structure, which macroscopically results in exceptional dimensional stability. At a synthesis time of 48 h as well as in a synthesis on a silicone membrane (7 d), this is not present. An explanation is provided by considering the fiber density, which in the case of a which in the case of a synthesis time of 72 h in the bioreactor is about four times higher than in the case of synthesis on a silicone membrane. Due to the limited oxygen supply, bacterial activity is reduced, which results in a less dense and thus mechanically less stable fiber network. less stable fiber network. The direct contact with oxygen during synthesis in the bioreactor thus results in a remarkable dimensional stability of the BNC vascular prostheses. When the synthesis time is longer than 72 h, the due to the extremely large native wall thickness in the rehydrated state pronounced wrinkling. For the synthesis of a BNC tube in the bioreactor a duration of 72 h and a rotational speed of 10 rpm at any diameter of the rotary diameter of the rotation profile are preferable.

TABLE 6.1
Wall thickness of BNC tubing in native and rehydrated state as well as mechanical
properties (breaking force, work) of the radial tensile test carried out
in the rehydrated radial tensile test carried out in the rehydrated state.
N corresponds to the number of synthesized BNC tubes and n is the total
number of ring samples generated from these tubes. ring samples.

| | | | | | |
|---|---|---|---|---|---|
| | | breaking | | | |
| Dₙₐₜᵢᵥ | D_{rehy} | force | work | | |
| in mm | in mm | in N | in mJ | n | N |

rotational speed
(48 h, Ø 8 mm)

| | | | | | | | |
|---|---|---|---|---|---|---|---|
| 2 | rpm | 0.42 ± 0.13 | 0.04 ± 0.02 | 2.46 ± 0.26 | 2.50 ± 0.58 | 14 | 2 |
| 10 | rpm | 0.85 ± 0.21 | 0.08 ± 0.08 | 6.02 ± 1.26 | 7.21 ± 2.03 | 32 | 3 |
| 18 | rpm | 1.14 ± 0.16 | 0.13 ± 0.04 | 6.48 ± 1.80 | 13.20 ± 6.39 | 36 | 3 |

diameter
(48 h, 10 rpm)

| | | | | | | | |
|---|---|---|---|---|---|---|---|
| 5 | mm | 0.94 ± 0.32 | 0.09 ± 0.03 | 7.90 ± 0.37 | 11.92 ± 3.09 | 29 | 2 |
| 8 | mm | 0.85 ± 0.21 | 0.06 ± 0.08 | 6.02 ± 1.26 | 7.21 ± 2.03 | 32 | 3 |
| 10 | mm | 0.91 ± 0.18 | 0.08 ± 0.03 | 7.72 ± 0.45 | 8.88 ± 4.42 | 16 | 2 |

duration
(10 rpm, Ø 8 mm)

| | | | | | | | |
|---|---|---|---|---|---|---|---|
| 48 | h | 0.85 ± 0.21 | 0.06 ± 0.03 | 6.02 ± 1.26 | 7.21 ± 2.03 | 32 | 3 |
| 60 | h | 1.31 ± 0.14 | 0.08 ± 0.03 | 6.12 ± 1.08 | 9.31 ± 2.14 | 40 | 2 |
| 72 | h | 2.56 ± 0.24 | 0.06 ± 0.01 | 8.40 ± 0.40 | 20.94 ± 3.33 | 20 | 2 |
| 144 | h | 4.79 ± 0.83 | 0.17 ± 0.02 | 17.05 ± 3.05 | 64.47 ± 12.51 | 20 | 2 |

silicon hose

| | | | | | | |
|---|---|---|---|---|---|---|
| 7 d, Ø mm | 1.38 ± 0.07 | 0.12 ± 0.01 | 4.61 ± 1.23 | 12.84 ± 3.52 | 14 | 2 |

### Example 11—Development of a Stent Graft with BNC Membrane

The term stent graft refers to a prosthesis consisting of a stabilizing metal framework (stent made of cobalt chromium or nitinol) and a synthetic vascular prosthesis (usually polymer; PET/ePTFE). In vascular surgery, stent grafts are used for the restoration of blood flow in coronary and peripheral arteries and for the treatment of aneurysms. In addition to bare metal stents and drug eluting stents, stent grafts are widely used in endovascular therapy because the membrane acts as a physical barrier to prevent the ingrowth of neointimal tissue into the vessel lumen, thus reducing the need for reintervention due to intimal hyperplasia or stent thrombosis. The membrane made from a mostly synthetic or electrospun polymer shows however, due to a lack of hemocompatibility, shows a tendency to thrombosis and thus inducing failure of the prosthesis. Several research groups are therefore focusing on the approach of a membrane made of a biological material, which is characterized biocompatibility and the ability to accelerate endothelialisation. For example, animal studies are already being of a stent graft using the membrane of bovine and ovine collagen as well as pericardial tissue are being performed. The additional possibility BNC membrane as a carrier for antiproliferative drugs or to accelerate endothelialisation is a promising approach for the use of BNC as a biomaterial. In the context of this work, a first prototype of a stent graft with BNC membrane is developed by dynamic synthesis in a bioreactor.

### Fabrication of the Stent Graft

For the fabrication of a stent graft with a BNC membrane, self-expanding Nitinol stents with a length of 40 mm and a diameter of 8 mm are used. The stent is placed on the respective profiled body prior to synthesis in the bioreactor. In general, in the following a distinction is made between the synthesis of a BNC membrane only on the outside of the stent and a double-sided, i.e. both inside and outside, membrane. For the synthesis of the outer membrane, a cylindrical rotation profile with a diameter of 7.6 mm is used. In order to generate a bilateral membrane is generated, a profile body with an offset of smaller diameter (5.6 mm) is designed. Here, the stent has direct contact with the rotational profile only at the ends (contact surface of the rotation profile 95.5 mm²). The rotation profile for the synthesis of a bilateral BNC membrane consists of two partial bodies connected by a square connector (2 mm×2 mm) to allow removal of the stent from the profile directly after stent from the profile directly after BNC synthesis. The parameters for cultivation in the bioreactor and post-treatment are based on those of the vascular prostheses from Example 10. The post-treatment drying (under rotation in the bioreactor), rehydration and final laser cutting of the stent graft takes place with a BNC membrane synthesized only externally on the rotation profile takes place. In this case, the profile body is manually removed at the end of the processing prior to before cleaning. In the case of a stent coated on both sides with BNC the rotational profile is removed in the native state, otherwise the BNC membrane would be induces deformation of the stent during drying.

### Characterization

The stent grafts with a BNC membrane synthesized in the bioreactor are visually evaluated with a stereomicroscope. In addition, crimping tests are performed to a diameter of 3 mm in the rehydrated state to verify the integrity of the membrane after crimping. In this context, the orifice diameter of the stent before and after crimping will be quantified to exclude a reduction of the vessel lumen after implantation. The BNC outer membrane in unilaterally sheathed stent grafts will be radial mechanical loading after rehydration is removed from the stent and measured by tensile testing. Visual inspection of fully processed, rehydrated stent grafts with a BNC membrane shows an exceptionally homogeneous coating of the stent struts. No irregularities or gaps are discernible and the method provides an outstandingly homogeneous wall thickness of the BNC membrane. In addition, the sheath sits stably on the stent due to radial compression during drying. The double-sided membrane on the inner and outer side appears significantly more opaque than the outer membrane alone. The microscopic images confirm the homogeneous envelope of the stent struts. The integrity of the BNC membrane is already evident in the native state after synthesis and remains intact during the entire processing up to the final rehydrated state, rehydrated state. Even radial crimping to a diameter of 3 mm does not lead to any damage of the BNC membrane with one and both sides of the stent struts. This is also confirmed by electron micrographs of the topography of the BNC membrane. The radial tensile test is performed on ring specimens of the BNC outer membrane, which is removed from the stent after complete processing. It no significant difference in rupture force or work compared to tubing made from BNC tubing synthesized without the stent structure in the bioreactor. The direct synthesis of the BNC membrane on the stent struts does not have a negative effect on the mechanical strength of the BNC. The orifice diameter of the stents is measured before crimping (DC0). immediately afterwards (DC1) and after one week of storage in a thermal oven at 37° C. (DC2). is determined. Six measurements of the diameter are performed per stent on opposite stent struts. The reference is a stent without BNC membrane. Both the BNC outer membrane and the bilateral both sides do not lead to a significant reduction of the orifice diameter. Overall. the wrapping of the stent struts with a BNC membrane proves to be a promising concept for the realization of a physical barrier to prevent potential restenosis by proliferation into the vessel lumen.

### Example 12—Three-Dimensional Tissue Component Made of BNC for the Percutaneous Aortic Valve Replacement

The treatment of severe aortic valve stenosis is usually minimally invasive with the with the replacement of the native aortic valve. This catheter-assisted aortic valve implantation (transcatheter aortic valve replacement, TAVR for short) is performed with a biological prosthetic heart valve, where the tissue component is usually a xenograft of porcine or bovine tissue, which is attached to a metal stent. TAVR is basically divided into balloon-expandable balloon-expandable and self-expanding prostheses. In the latter, a stent (e.g., nitinol alloy) is used, into which the tissue component consisting of six sutured individual parts is sewn into the stent. The three leaflets cause the valve function of the implant through opening and closing processes. The three leaflets are fixed immovably to the inside of the stent. The conventional manual suturing process, which involves hundreds of individual surgical knots, is extremely error-prone, time-consuming and costly. In addition, sutures form potential mechanical weak points that induce implant failure. A sutureless, three-dimensional tissue component of BNC for TAVI prostheses is fabricated using the bioreactor described herein. For the production of the three-dimensional tissue component from BNC in the bioreactor, a modified profiled body (also referred to as shaped body or shaped article) is used, on the surface of which, under constant BNC synthesis takes place under constant rotation. In order to ensure sufficient stability of the three leaflets, the synthesis is carried out in the bioreactor with a duration of 4 d. The drying of the native, three-dimensionally shaped BNC takes place for 24 h on the profiled body under rotation in the bioreactor. After complete drying, a three-dimensional laser cut of the skirt and leaflet edges is performed. The laser cut in dry condition offers the advantage, that slipping of the fabric component on the profile body is prevented. After rehydration, the three-dimensional BNC component is removed from the profiled body and is fixed in the stent after processing steps such as cleaning and sterilization.

For the evaluation of the prototype TAVI with a three-dimensional BNC tissue component a self-expanding nitinol stent was used. The tissue component is fixed by an internal second stent. This allows free movement of the leaflets and supports the and the skirt area is supported. The completely sutureless, three-dimensional BNC tissue component is characterized by an exceptionally homogeneous material structure. The defined shape of the rotation profile allows the complex leaflet complex leaflet geometry, resulting in the formation of inwardly directed semilunar pockets are formed. In addition to visual inspection, the closing characteristics of the three-dimensional BNC component is evaluated using a simplified flap closure test. For this purpose, valve closure is generated in vitro using a column of fluid. The prosthesis is placed in a 3D-printed holder with a diameter (26 mm) corresponding to the intended diameter of the implanted TAVI prosthesis. A plastic pocket serves as the fluid reservoir and allows a fluid column of approximately 10 cm above the mounted prototype. A complete and symmetrical closure of the valve prosthesis can be observed. The synthesis of a three-dimensional BNC tissue component in the bioreactor thus represents an innovative, promising concept for the fabrication of a functional TAVI prosthesis with a completely sutureless tissue component.

### Example 13—Prevention of Paravalvular Leakage in Aortic Valve Prostheses with Locally Swellable BNCs

One of the most frequent postoperative complications of a percutaneous aortic valve replacement is leakage between the vessel wall and the prosthetic valve due to calcification or due to calcification or incomplete adherence of the prosthesis to the aortic annulus. This aortic insufficiency is also known as paravalvular leakage (PVL) and leads to a 2 to 3 times higher mortality. The PVL induces a non-physiological backflow (regurgitation) of blood from the aorta into the left ventricle of the heart. With first-generation prosthetic valves, paravalvular insufficiency occurred in 15% to 20% postintervention. The design of newer prostheses takes into account, besides the goal of smaller catheter diameters and better positioning of the valve, the design of newer prostheses takes into account the avoidance of PVL through components made of plastic (polyester, PET) or biological tissue (porcine tissue) additionally biological tissue (porcine pericardium). The rate of moderate and severe insufficiencies has been reduced to 3% to 7%. Mild forms of paravalvular regurgitation occur even with novel valve designs, however, occur in approximately 30% of cases. In the context of the present a method for the fabrication of a TAVI prosthesis with a tissue component of BNC, whereby the skirt is characterized by defined local swellable areas for sealing potential paravalvular insufficiencies. For the fabrication of locally swellable BNCs, a stabilization of the material in combination with a pressing process is performed. The stabilization is carried out with glycerol or PEG400 according to the process described above in order to counteract hornification of the material during drying and thus to ensure that the material does not swell during rehydration. After complete storage of the stabilizer solution, the stabilizer solution is displaced at defined points in a pressing process with a 3D-printed molded body, thus specifically inducing local hornification of the BNC. In these hornification areas the swelling process is prevented during rehydration. The subsequent drying takes place at room temperature (23° C.) for about 48 hours. The incorporated stabilizers are partially rinsed out again during rehydration, which results in defined swelling of the material according to the geometry of the die. Finally, cleaning with aqueous NaOH and a sterilization process take place. It should be noted that the process steps stabilizing and pressing can also take place in reverse order. In this alternative process variant, the native BNC is pressed into the 3D-printed PLA mold and then the stabilization solution is pipetted into the recess. The swelling behavior of the biomaterial BNC using stabilizers has already been explained above. For the stabilization, analogous concentrations of 5%, 10% and 20% are used for stabilization. During the pressing process, the BNC (40 mm×40 mm) is placed between two pressing plates on a foil. The grid-like 3D press mold (PLA) built up from struts (width 1 mm) and (5 mm×5 mm) is placed on the surface of the BNC. For a homogeneous pressure distribution, an additional 3D-printed solid body (70 mm×70 mm) and a silicone plate (Shore hardness 50) are placed on the mold. Due to the rigid pressing plate in combination with the partially hollow press mold the BNC areas with stabilizer solution are created locally, which show unidirectional swelling behavior. The pressing pressure is applied starting with 2 N/mm² for 30 s to 5 N/mm² (5 min) and finally to 10 N/mm² (5 min) iteratively increased to avoid damage to the BNC by the compression mold. Observation of the specimens after rehydration shows locally defined, clearly defined swelling in the area of the square recesses of the compression mold. The higher the stabilizer concentration, the stronger is the swelling capacity. A thickness analysis differentiated between struts and squares confirms this visual impression. The swelling factor is calculated from the quotient of the rehydrated thickness of the squares and the strut thickness.

It can be seen that a doubling of the stabilizer concentration is accompanied by a doubling of the swelling factor. There are no differences between the stabilizers PEG400 and glycerol. The strut thickness is always below 100 μm, regardless of the concentration of the stabilizer solution. The swelling capacity or thickness of the stabilized squares increases steadily with increasing concentration. The process for producing locally swellable BNCs thus enables a defined, locally delimited swelling capability of any geometry depending on the selected concentration of the stabilization solution. The local swellability of the BNC explained in the previous section will now be adapted to the geometry of a TAVI prosthesis. For this purpose, in the first step a modified 3D compression mold is created, which produces local hornification in the area of the stent struts and ensures circular, swellable areas in the interstices. The latter serve as a preventive measure outside the stent due to the annulus-sealing property outside the stent to prevent postoperative paravalvular leakage. PEG400 with a concentration of 20 wt % is used to stabilize the swellable areas. The BNC component is sewn into the stent after drying. Subsequently, a crimping procedure is performed in the dry state for 72 h. Visual inspection after crimping and rehydration shows no damage to the material structure. The swellable areas are intact and their functionality is not restricted. In addition to visual inspection and evaluation of the integrity of the skirt component after crimping, two further aspects relevant for TAVI prostheses are highlighted, aspects relevant for TAVI prostheses. First, the time course until complete swelling is crucial for the sealing function, since complications caused by PVL usually occur in the first four weeks after implantation. For this purpose the thickness of the swellable areas of the skirt component at different points in time. There is a rapid increase in thickness on contact with water, which reaches the maximum value after 1 h. The thickness of the skirt component also increases after 21 d of storage. Even after a storage period of 21 d, this value no longer changes significantly. On the other hand, the aperture diameter of the stent should not affect the vessel lumen due to the vessel lumen due to the local swelling in the skirt area. For this purpose, the diameter of the stent diameter of the stent at an implantation diameter of 22 mm is determined. The stent is placed in a custom 3D-printed device, which is filled with water. In addition, the device simulates the device simulates a simple leakage. Generally, swelling only occurs in the area of the leakage, swelling of the BNC occurs. The radial force exerted by the stent the radial force exerted by the stent, swelling along the simulated aortic annulus in the device is prevented. After different time points, the corresponding opening diameter of the stent is determined. It can be seen that there is no reduction in the orifice diameter compared to the dry state. Even after one week of storage, the diameter remains unchanged. Thus, overall, the application of locally swellable BNC to the skirt component of the TAVI prosthesis proves to be an innovative concept in the context of prevention of paravalvular leakage in percutaneous aortic valve replacement.

The knowledge gained in the previous section from the simplified skirt component is now integrated into the conventional skirt geometry of a TAVI prosthesis. For this purpose, the 3D compression mold is modified once again three skirt components with three locally swellable areas each are produced. In addition, three leaflets are generated according to the standard process. The geometry for skirt and leaflet is cut out in the dry state with the laser (CO₂ laser, Epilog Zing 24, Epilog Zing). Laser cutting can also be performed in the rehydrated state if required. After suturing the six components, skirt and leaflet, the entire BNC fabric component is fixed in the stent. A TAVI prosthesis is thus with a BNC tissue component and locally swellable skirt. The locally swellable areas clearly protrude visibly beyond the diameter of the stent and were created in this prototype by stabilization with 20 wt % PEG400. The three leaflets appear in their natural alignment and ensure complete closure in the valve closure test. Furthermore, a symmetrical closure behavior of the three segments is evident. The functionality of the implant is enhanced by the locally swellable areas to sealing of potential PVL. In addition, the method allows reduction of potential PVL without the use of additional components on the outside of the skirt. Since swelling occurs only after implantation, the catheter diameter is thus not adversely affected. Overall the prototype with a complete BNC component shows the successful integration of the concept of local swelling of the BNC in the manufacturing process of TAVI prostheses.

In summary, several concepts for the use of the biomaterial BNC in cardiovascular implants is described. The functionality of the prototypes shows the outstanding potential of the biomaterial BNC for an application in the clinical field. It has been shown that hygroscopic substances (glycerol, polyethylene glycol) can be introduced into the fiber network, so that the mechanical force response of the BNC is systematically changed under loading. Manufacturing methods for bacterial nanocellulose are described. A described bioreactor enables the synthesis of a three-dimensionally shaped BNC. This enables vascular prostheses and stent grafts with a membrane made of BNC as well as a three-dimensional, sutureless tissue component made of BNC for transcatheter aortic valve prostheses. Furthermore, the production of locally swellable BNC, which can be used for the prevention of potential paravalvular leakage in minimally invasive implantable prosthetic heart valves. The application of this concept in aortic valve prostheses is achieved by the fabrication of an aortic valve prosthesis with a locally swellable tissue component of BNC.

The use of bacterial nanocellulose, dried bacterial nanocellulose, pressed a dried bacterial nanocellulose, stabilized dried bacterial nanocellulose, rehydrated bacterial nanocellulose in medicine, biomedicine, cosmetics, packaging industry, paper industry, pharmaceutical industry, water treatment, filtration of fluid media, electronics or sensor technology is described.

In particular, the use of bacterial nanocellulose, dried bacterial nanocellulose, pressed an dried bacterial nanocellulose, stabilized dried bacterial nanocellulose, rehydrated bacterial nanocellulose in a vascular graft, a vascular prosthesis, a medical implant, a vascular implant, a stent, a stent graft, a cover for cardiac pacemakers, a cardiac valve(s), a venous valve(s), a heart valve (prosthesis), an aortic valve (prosthesis), in a medical occluder, in a tissue occluder, in a tissue patch, as drug coating, or in a biosensors is described.

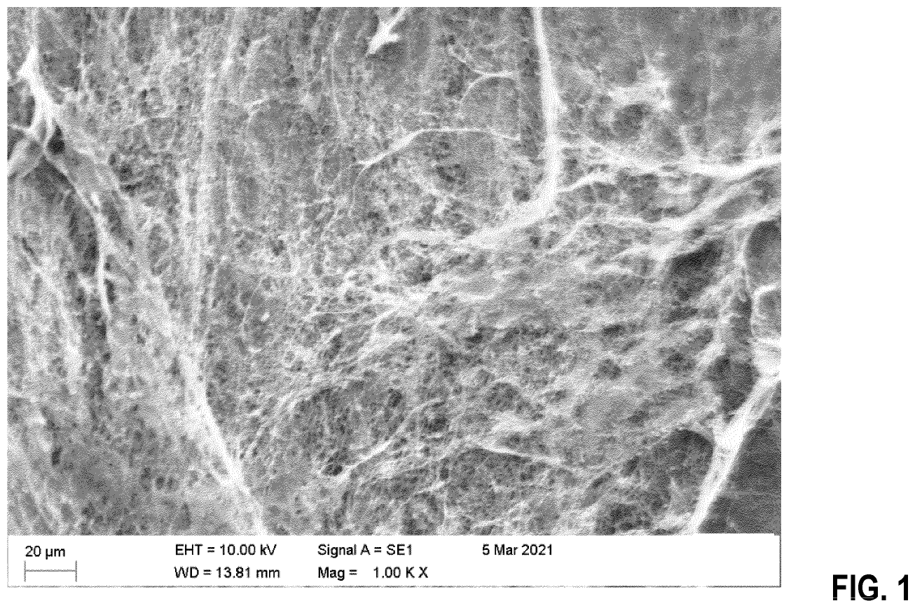

FIG. 1 shows an electron microscope picture of a prior art bacterial nanocellulose sheet obtained by process according to example 1 in a top view, wherein the bacterial nanocellulose was obtained in silicone molds.

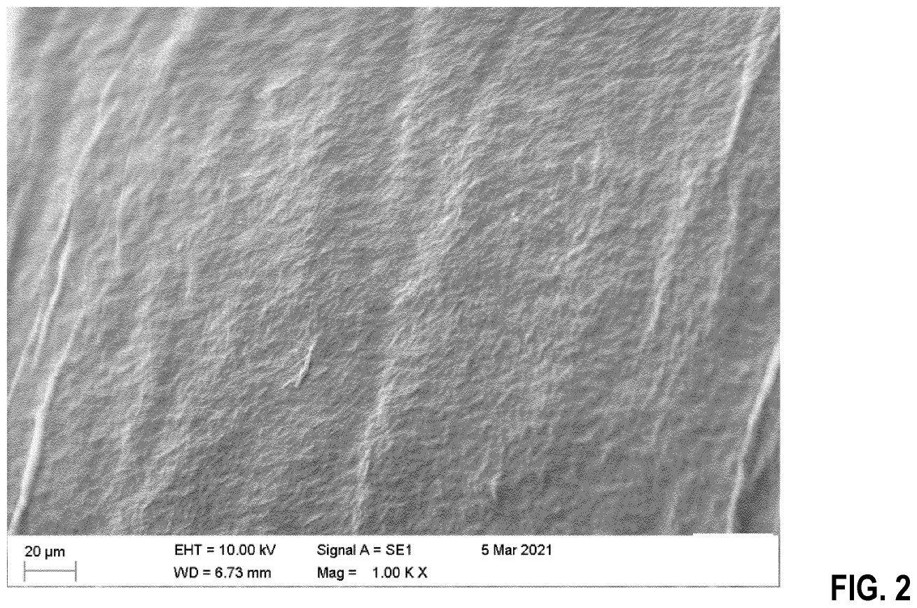

FIG. 2 shows an electron microscope picture of a bacterial nanocellulose sheet obtained by process according to example 2 in a top view, wherein the bacterial nanocellulose was produced by using a PEEK rod. In comparison to FIG. 1 it can be seen that the bacterial nanocellulose of example 2 has a higher fiber density than the bacterial nanocellulose of example 2.

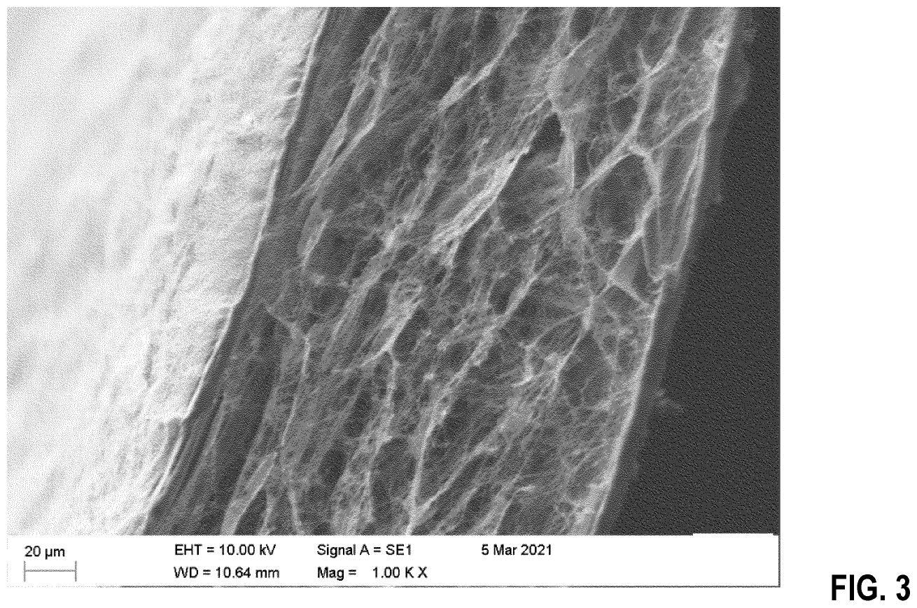

FIG. 3 shows an electron microscope picture of a prior art bacterial nanocellulose sheet obtained by process according to example 1, in a side view, wherein the bacterial nanocellulose was obtained in silicone molds.

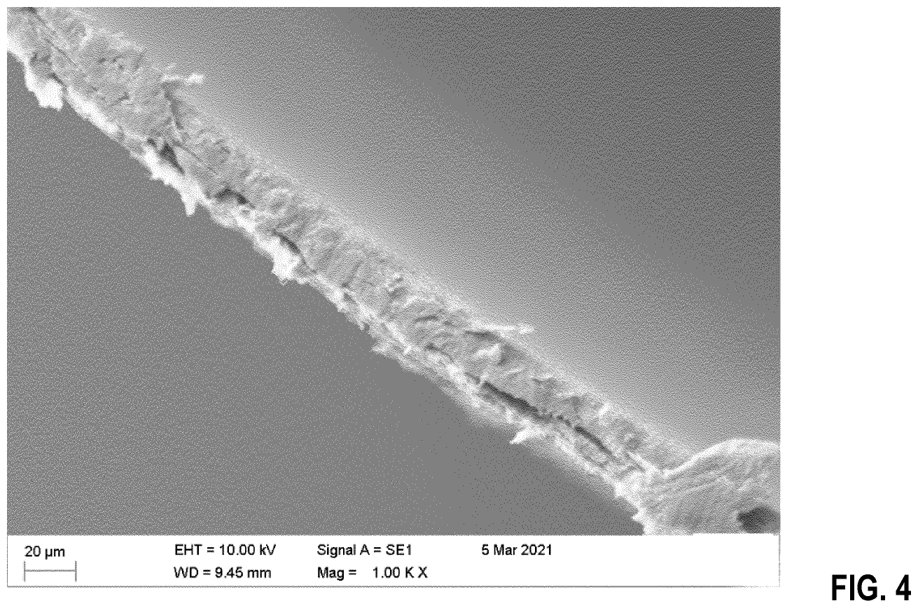

FIG. 4 shows an electron microscope picture of a bacterial nanocellulose sheet obtained by process according to example 2 in a side view, wherein the bacterial nanocellulose was produced by using a PEEK rod. In comparison to FIG. 3 it can be seen that the bacterial nanocellulose of example 2 has a higher fiber density than the bacterial nanocellulose of example 1.

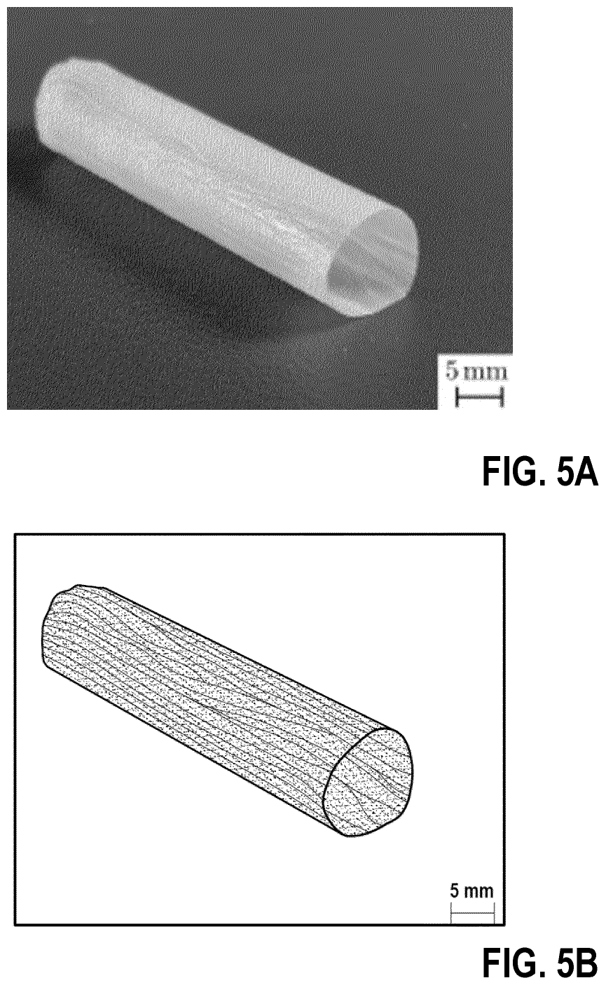

FIG. 5A shows a photo of a dried and rehydrated tubular shaped bacterial nanocellulose. FIG. 5B shows the schematic drawing of the photo of 5A.

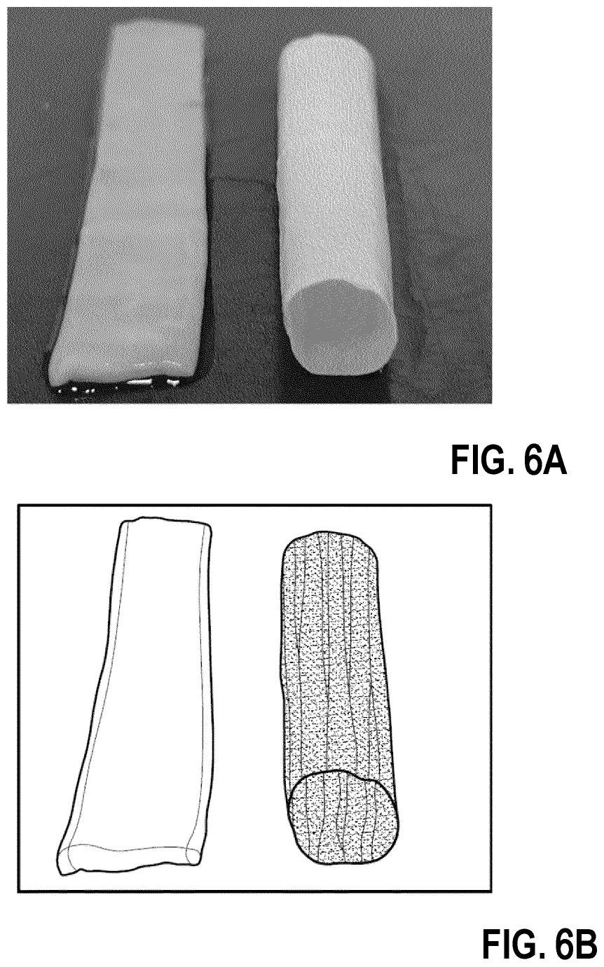

FIG. 6A shows a photo comparing of the bacterial nanocellulose obtained according example 1, being a collapsed hollow cylinder on the left side and example 2b a form stable hollow cylinder on the right side, both being dried and rehydrated. The bacterial nanocellulose according to example 1 collapses whereas the bacterial nanocellulose according to example 2b has a stable shape. The BNC of example 1 was synthesized in a silicone hose and does not show any dimensional stability even after a synthesis time of 7 d. since the oxygen supply through the silicone membrane is limited. The bacterial nanocellulose obtained in example 2b (using a rotating PEEK rod) has a higher inner mechanical stability and a higher mechanical strength than the bacterial nanocellulose according to Example 1. FIG. 6B shows the schematic drawing of the photo of 6A.

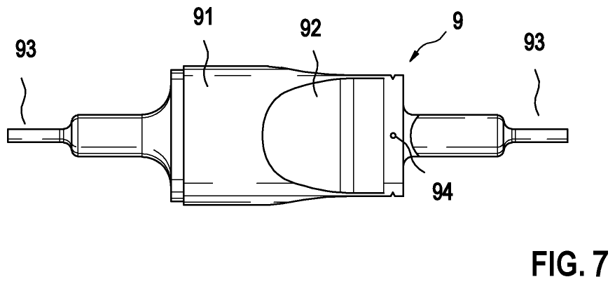

FIG. 7 shows a schematic drawing of a shaped article 9 for making a heart valve prosthesis. The shaped article comprises holding sections 92 for mounting the shaped article in a holder of a bioreactor. The shaped article comprises a skirt section 8 having a circular shape and leaflet sections 92 having the shape of a heart valve leaflet. The shaped article can further comprise a focus point for laser cutting. On this shaped article, bacterial nanocellulose can be grown. The shaped article is preferably made of PEEK.

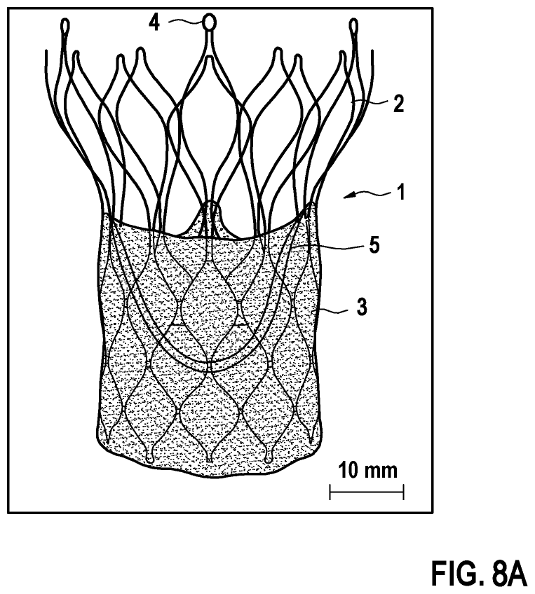

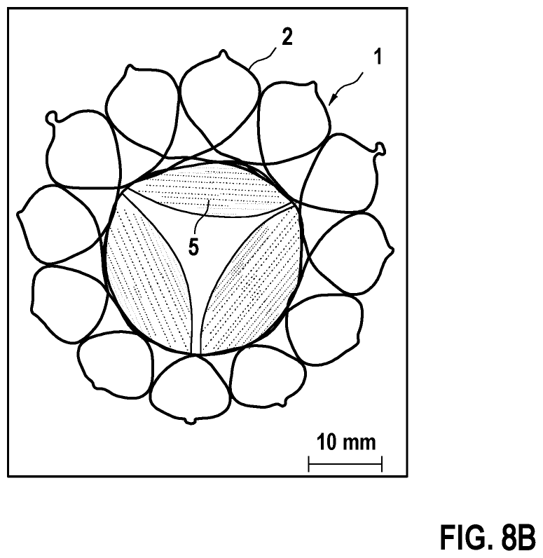

FIG. 8A shows a schematic drawing of a transcatheter heart valve prosthesis 1 comprising the bacterial nanocellulose according to the invention in a side view. The transcatheter heart valve prosthesis 1 comprises a stent base body 2 comprising metallic struts. An outer skirt 3 (also referred to as peripheral sealing shell) and/or an inner skirt (not visible) is made of the bacterial nanocellulose and is fastened on the stent base body 2 e.g. by gluing or suturing using thread, for example a polytetrafluoroethylene thread. The outer skirt and/or the inner skirt 3 is adjoined by heart valve leaflets 5, for example three heart valve leaflets, which can be made of bacterial nanocellulose or pericardial tissue.

FIG. 8B shows a schematic drawing of the heart valve prosthesis comprising the bacterial nanocellulose of FIG. 8A in a top view. The heart valves 5, for example the three heart valve leaflets, are fixed on the stent base body 2. For example, the heart valves 5 may be formed of bacterial nanocellulose sheets or pericardial tissue, each of which opens or closes according to the blood flow forces acting thereon.

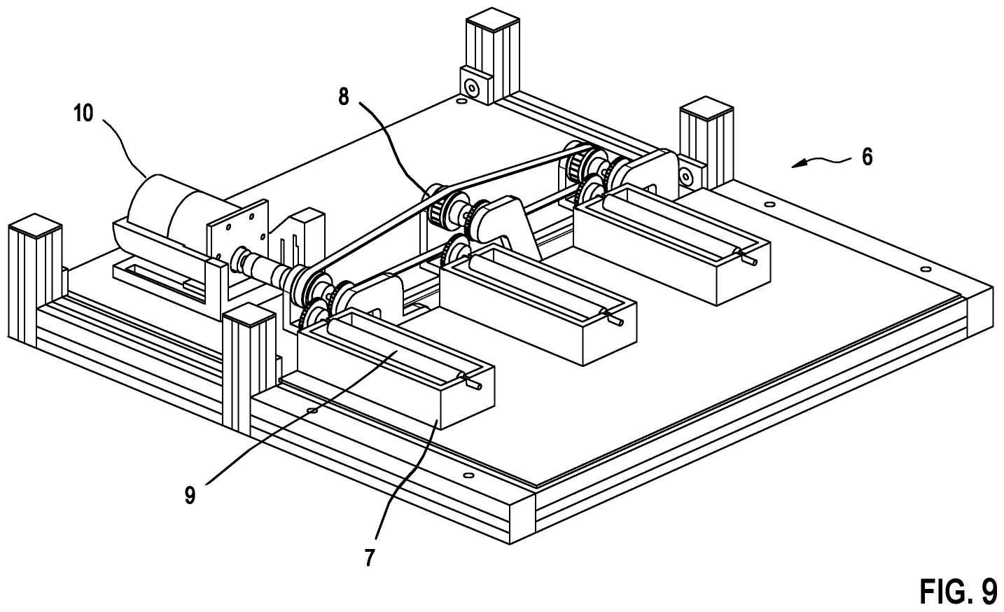

FIG. 9 shows a schematic drawing of a device 6 for producing bacterial nanocellulose. The device comprises at more than one (here three) culture vessels 7 for receiving a medium for bacterial nanocellulose producing bacteria, in which the bacterial nanocellulose can be generated. The device further comprises more than one (here three) rotating units 8 for rotatably mounting a shaped article 9, respectively. The device comprises more than one shaped article 9 each being rotatably mounted by one rotating unit 8. The rotating unit 8 is based on a belt drive, which is driven by a motor 10, thereby rotating each shaped article within its respective culture vessel.

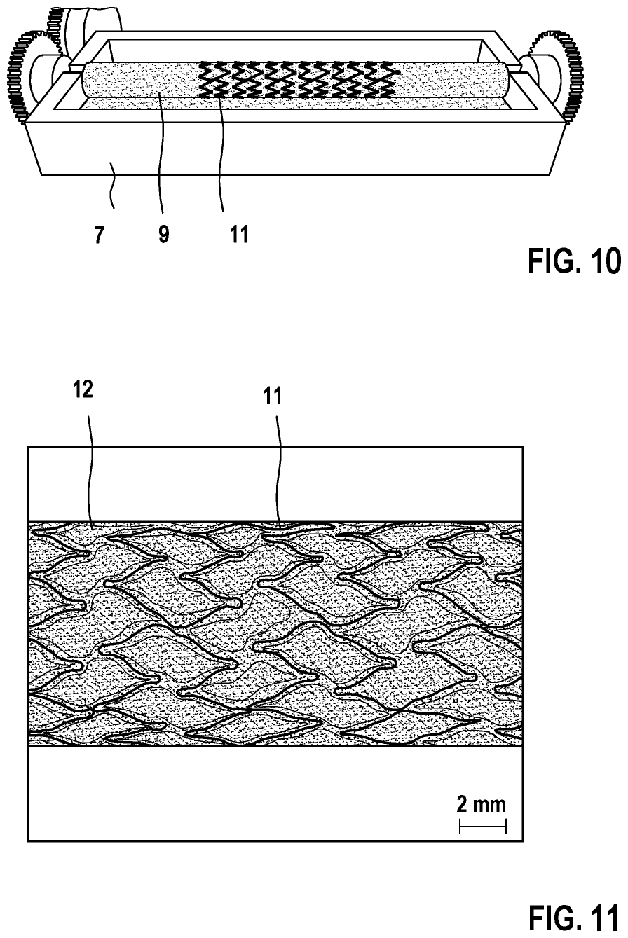

FIG. 10 shows a detailed schematic depiction of a device for producing bacterial nanocellulose showing one culture vessel 7 for receiving a medium for bacterial nanocellulose producing bacteria and for receiving one shaped article 9. The shaped article 9 is rotatably mounted by one rotating unit 7. The rotating unit 7 can be driven by a motor (not shown), thereby rotating the shaped article within the culture vessel. The shaped article is rod shaped and comprises on its surface a stent 11. When rotating the shaped article together with the stent structure a covered stent can be obtained, where the stent structure is covered by the obtained bacterial nanocellulose.

FIG. 11 shows a schematic drawing of a stent 11 covered with bacterial nanocellulose 12. The struts of the stent are partially embedded within the cellulose. The stent may be made from nitinol struts. The stent may have a diameter of 7.6 mm and a length of 87 mm.

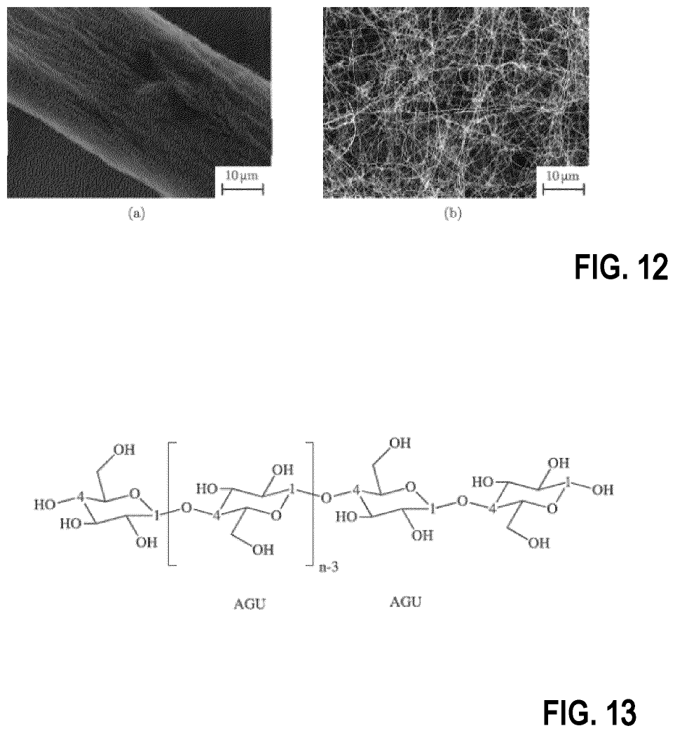

FIG. 12 shows SEM images comparing the size range of fibers of (a) plant cellulose and (b) bacterial nanocellulose of the bacterial strain K. hansenii. Bacterial nanocellulose fibres of the bacterial strain K. hansenii. Are in a range of 30 nm to 60 nm.

FIG. 13 shows the molecular structure of the bacterial nanocellulose consisting of adjacent anhydroglucose (AGU) units. These AGU units are covalently linked to each other via β-1,4-glycosidic bonds. Ring-shaped glucose monomers are covalently bonded to each other via a polycondensation reaction between the hydroxyl group on carbon atom C-1 of one glucose unit and C-4 of an adjacent glucose unit. Each glucose unit, also known as AGU, is alternately rotated about 180°. Two adjacent AGU units form the disaccharide cellobiose, which is considered the repeating cellulose unit. At the end of the Cl-carbon atom, cellulose exhibits a reducing function due to a rearrangement of the hydroxy group into an aldehyde group. At the other end (C-4) a non-reducing alcoholic hydroxy group is found. Depending on the origin, the chain length or degree of polymerization varies from 300 to 10,000 AGU, with all the properties of cellulose being present from 20 to 30 units on. Each AGU has a hydroxy group at carbon atoms C-2 (secondary), C-3 (equatorial) and C-6 (primary). Their partially positively charged hydrogen atoms form inter- and intramolecular hydrogen bonds due to the strong electronegativity of oxygen (FIG. 2.4). The hydroxy groups and bridging oxygens create a fiber composite with crystalline and amorphous domains.

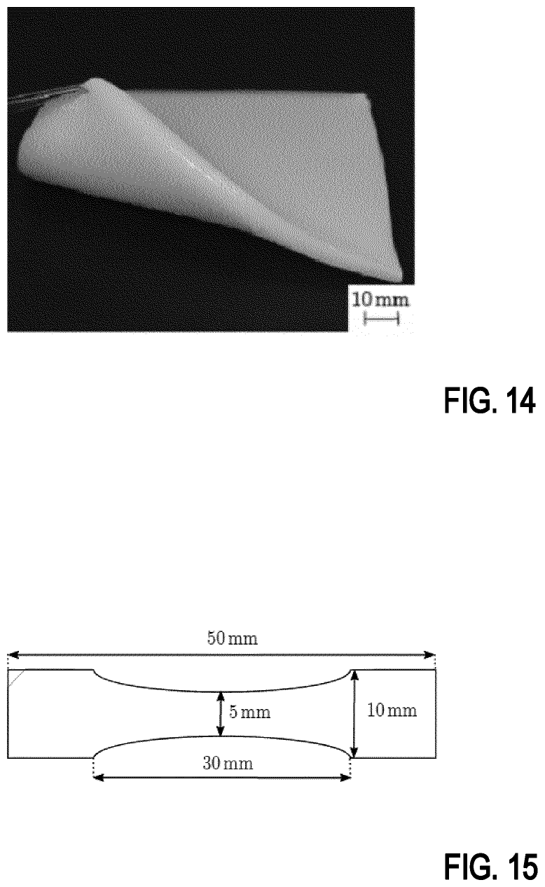

FIG. 14 shows a native BNC fleece obtained according to example 2a after a synthesis of 7 d.

FIG. 15 shows a specimen geometry for uniaxial tensile tests according to a modified DIN EN ISO 527-2 (type 1BA). The overall length of the BNC is 50 mm and the width of the grip section is 10 mm. The gauge section has a distance between shoulders is 30 mm and a width of 5 mm.

FIG. 16 shows schematic overview of the different process variants for the post-treatment of bacterial nanocellulose. The individual process steps are listed with the parameters investigated in each case.

FIG. 17 shows a force-elongation diagram in dependence of different drying methods of the BNC obtained according to Example 2a. GT means the BNC was freeze dried for 72 h, KS means the BNC was dried in a climate chamber at 23° C. an 10% rel. humidity. A noticeable increase of the initial modulus at higher drying temperature (in the oven at 100° C.) is observed. Elongation at break and force modulus do not show any significant differences, whereas the breaking force, compared to native specimens, increases significantly due to the drying process.

FIG. 18 shows a flow chart of a standard process for the production of a nonwoven from BNC. r.F. stands for relative humidity. The process comprises the following steps in a consecutive order:
1. BNC-Growth (Cultivation)
2. Drying the BNC
3. Pressing the dried BNC
4. Cleaning the dried and pressed BNC
5. Rehydrating the dried, pressed and cleaned BNC

The cultivation was done by using the growth medium comprised and the nutrient solution as described in example 2a under static conditions.

FIG. 19 shows force-elongation curves of BNC specimens having different relative humidities (r.F.)/water content. For dried BNC the relative humidity is 20%. For re-hydrated BN the relative humidity is 70%. For native BNC the relative humidity is 98%. The aggregation of fibers and additional hydrogen bonds formed during drying lead to a significantly higher breaking strength (breaking force) and a higher F-modulus (F-Modul) than in native or rehydrated samples.

FIG. 20 shows a flow chart of a standard stabilization process for the post-treatment of BNC to obtain stabilized BNC. r.F. stands for relative humidity. The process comprises the following steps (in a consecutive order):
1. BNC-Growth (Cultivation)
2. Stabilization of the BNC
3. Drying of the stabilized BNC
4. Pressing the stabilized and dried BNC optionally cleaning the stabilized, dried and pressed BNC (not shown in the flow chart)
5. Rehydrating the (cleaned) stabilized, dried and pressed BNC

Stabilization of the biomaterial with glycerol and PEG400 is performed after cultivation (static synthesis in an incubator (7 d, 28° C.)) and a subsequent rinsing process with ultrapure water. The inclusion of the stabilizers thus takes place before the drying process, in order to prevent the formation of hydrogen bonds by the removal of water.

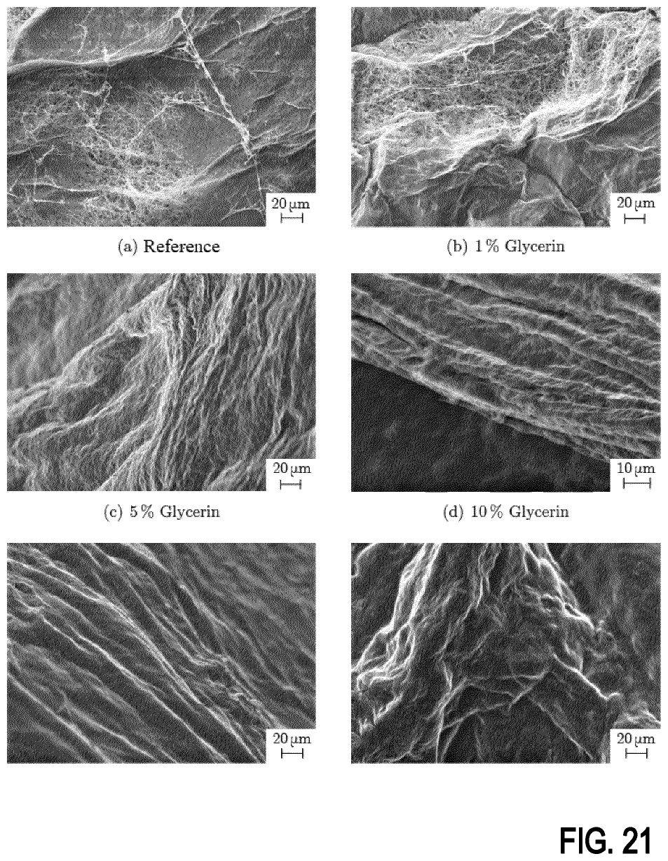

FIG. 21 shows electron microscope images of the coating of the BNC fiber network with glycerol at different concentrations of the Glycerin stabilizer solution.

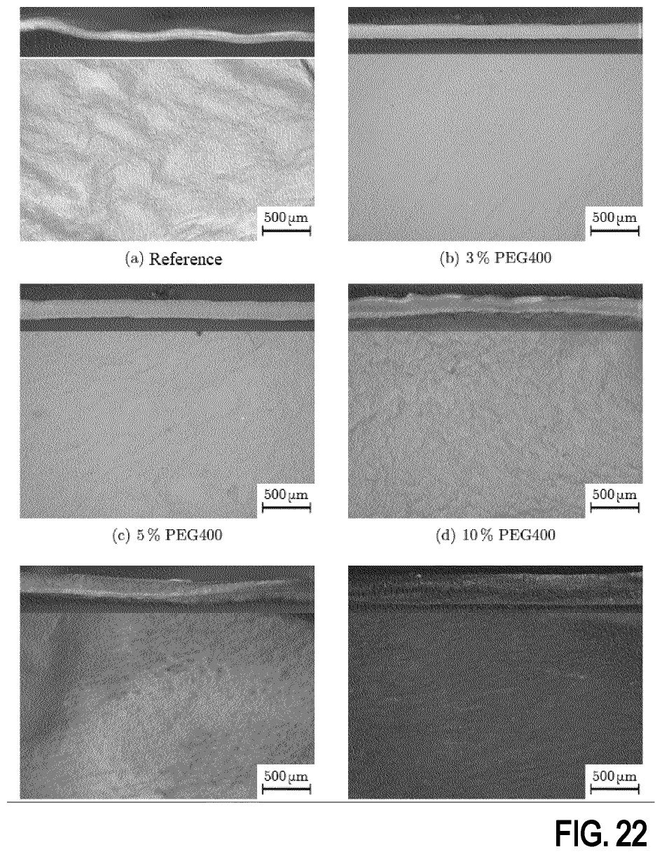

FIG. 22A, B, C show electron microscope images of BNC samples stabilized with PEG400 after cobalt thiocyanate staining. FIG. 22(a)-(c) show the cross-section of the sample and the two pictures at the bottom show the surface of the sample as a function of the stabilizer concentration.

FIG. 23 shows a plot of the swelling factor for stabilized BNC samples as a function of the stabilizer concentration (3 wt %, 5 wt %, 10 wt %, 20 wt % Glycerin or PEG400) after rehydration related to the thickness of the material in the pressed state. The reference represents a non-stabilized sample.

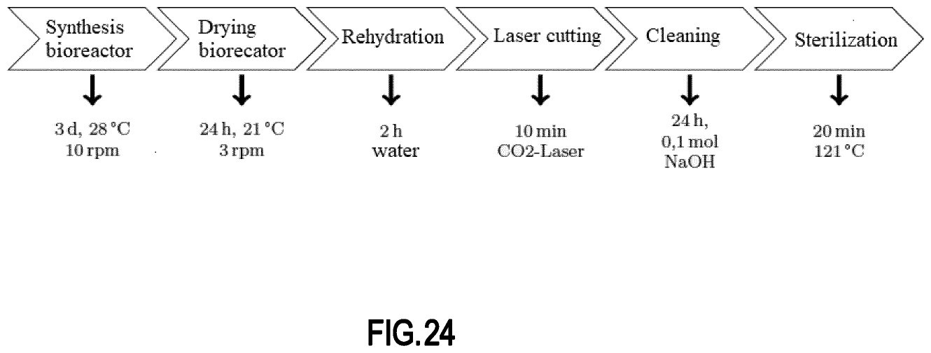

FIG. 24 shows a flow chart of a standard process for manufacturing a BNC tube. The process comprises the steps in a consecutive order:
1. BNC-Synthesis in a bioreactor (using a rotating rod shaped article)
2. Drying the BNC in the bioreactor (using the rotating rod shaped article)
3. Rehydration of the dried BNC
4. Laser Cutting of the rehydrated BNC
5. Cleaning the laser-cut BNC
6. Sterilization of the cleaned BNC

Such a BNC can be used as a vascular graft.

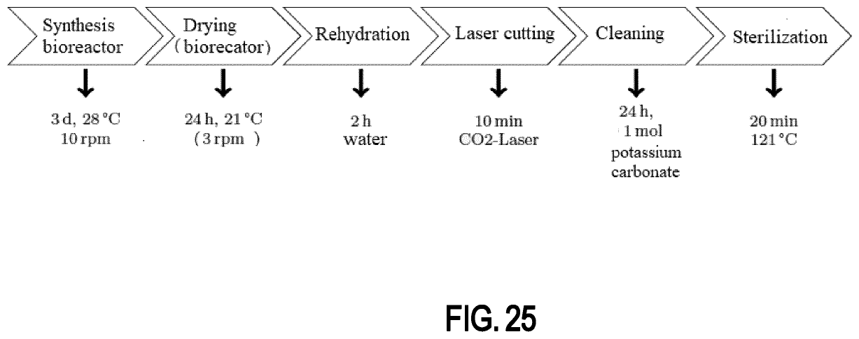

FIG. 25 shows a flow chart of a standard process for manufacturing a stent graft with a BNC membrane. The process comprises the steps in a consecutive order:
1. BNC-Synthesis in a bioreactor (using a rotating rod shaped article covered with a stent)
2. Drying the BNC
3. Rehydration of the dried BNC
4. Laser Cutting of the rehydrated BNC
5. Cleaning the laser-cut BNC
6. Sterilization of the cleaned BNC

The post-processing steps drying, rehydration and laser cutting of the stent graft can take place on the shaped article (under rotation in the bioreactor) in case the BNC is synthesized only on the outside of the stent. In case a stent is coated on both sides of the stent with BNC, the shaped article is manually removed prior to drying.

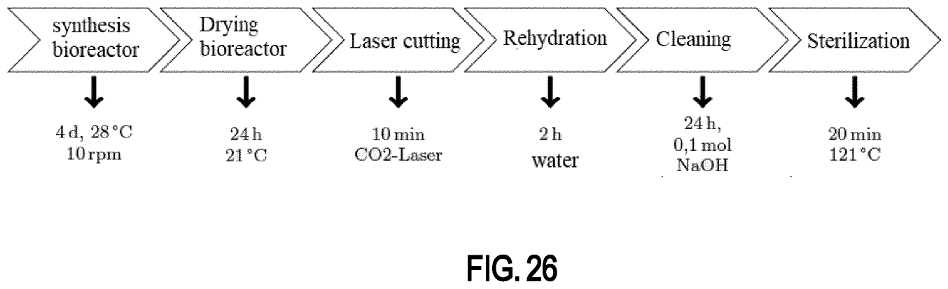

FIG. 26 shows a flow chart of a standard process for manufacturing a three-dimensional tissue component made of BNC (for percutaneous aortic valve replacement preferably using the shaped article of FIG. 7). The process comprises the steps in a consecutive order:
1. BNC-Synthesis in a bioreactor (using a shaped article)
2. Drying the BNC in the bioreactor
3. Laser Cutting of the dried BNC
4. Rehydration of the dried BNC
5. Cleaning rehydrated BNC
6. Sterilization of the cleaned BNC

Drying of the native, three-dimensionally shaped BNC takes place on a shaped article under rotation in the bioreactor. After complete drying, a three-dimensional laser cut of the skirt and leaflet edges is performed. The laser cut in dry condition offers the advantage, that slipping of the fabric component on the shaped article is prevented. After rehydration, the three-dimensional BNC component is removed from the shaped article and, after final processing steps such as cleaning and sterilization.

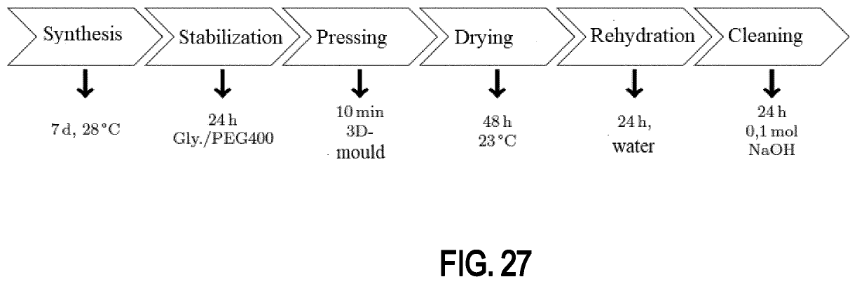

FIG. 27 shows a flow chart of a standard process for manufacturing locally swellable BNC.

The process comprises the steps in a consecutive order:
1. BNC-Synthesis
2. Stabilization using Glycerin and/or polyethyleneglycol
3. Pressing the stabilized BNC in a 3D-mould
2. Drying the pressed and stabilized BNC
3. Rehydration of the dried, pressed and stabilized BNC
5. Cleaning the rehydrated BNC

It should be noted that in an alternative process the process steps of stabilization and pressing can also take place in reverse order (thus first pressing and then stabilization).

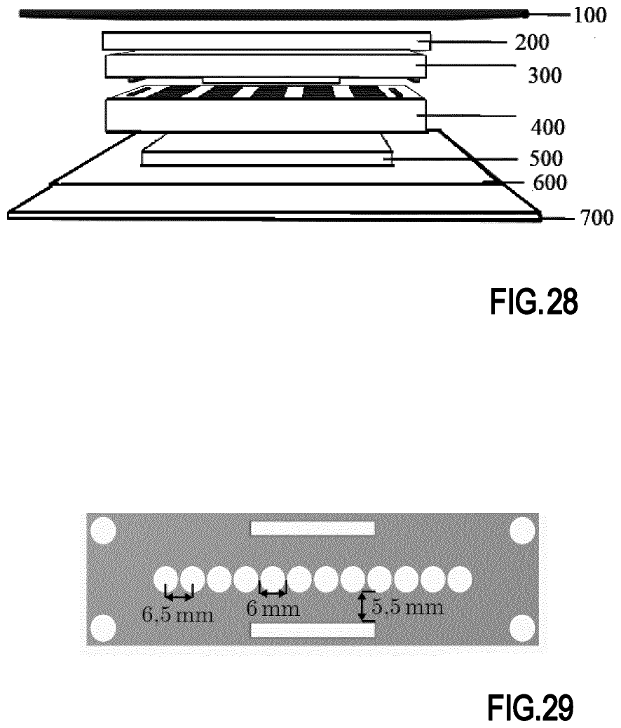

FIG. 28 shows a schematic representation of a pressing device for the production of locally swellable BNC. The pressing device comprises two press plates (an upper press plate 100 and a lower press plate 700), a 3D press mold 400, (optionally a solid body 300), a pressure compensation layer 200 and optionally a foil 600 on the lower press plate 700. The BNC 500 is located between two press plates 100, 700 on a foil 600. The 3D press mold 400 (e.g. made of PLA) is placed on the surface of the BNC 500. For homogeneous pressure distribution, a solid body 300 (e.g. made of PLA) and a pressure compensating plate 200 (Shore hardness of 50, e.g. a silicone mat) are also placed on the press mold 400. Due to the rigid press plates in combination with the press mold having recesses or openings, stabilized areas using a stabilizer solution can be generated locally in the BNC.

FIG. 29 shows a 3D press mold. The press 3D mold comprises recesses or openings for creating locally swellable areas. Such a press mold can be used to fabricate a TAVI skirt.

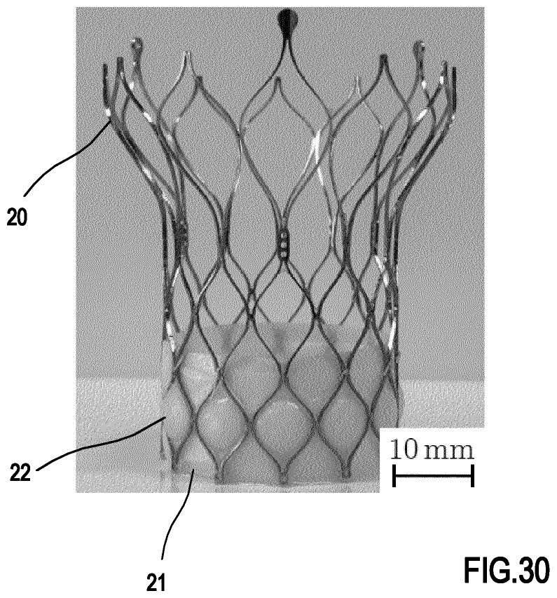

FIG. 30 shows a photo of a TAVI prosthesis 20 having a BNC skirt 21 with locally swellable areas 22 (here bumps which are located between the struts of the prosthesis). These locally swellable areas can offer a sealing function when being rehydrated (the photo shows the rehydrated state).

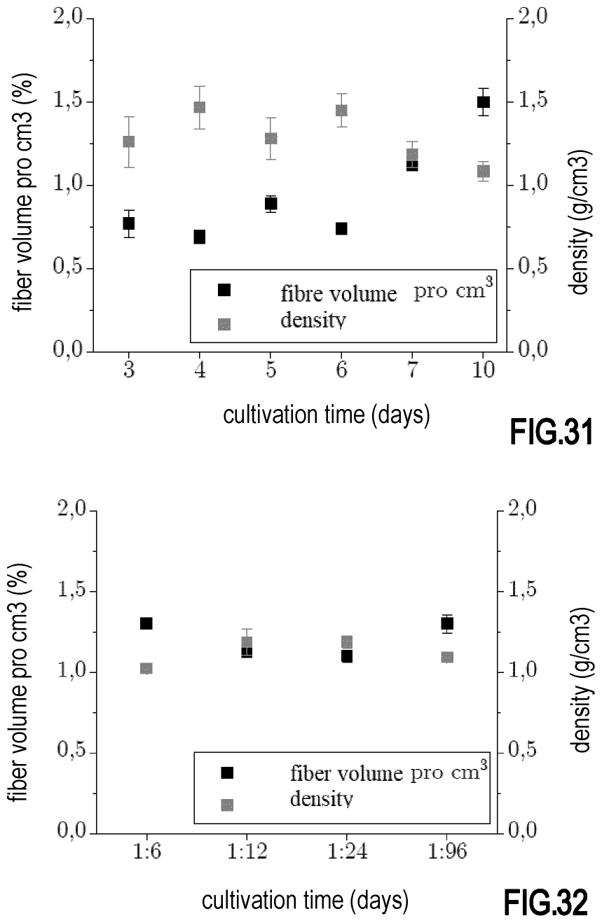

FIG. 31 shows a diagram showing the fiber volume and density of BNC as a function of cultivation time. The bacterial nanocellulose obtained from K. hansenii (cultivation time 3 days to 10 days) consists of nanocellulose fibers having a density in the range of 1.100 g/cm³ to 1.500 g/cm³, preferably 1.30±0.10 g/cm³.

FIG. 32 shows a diagram showing the fiber volume and density of BNC as a function of bacterial solution to nutrient solution. The bacterial nanocellulose obtained from K. hansenii (bacterial solution: nutrient solution is 1:6 to 1:96) consists of nanocellulose fibers having a density in the range of 1.0 g/cm³ to 1.35 g/cm³. The density obtained using a bacterial solution: nutrient solution of 1:18 (see example 2a) is 1.30±0.10 g/cm³.

1. A method for producing a shaped element made of bacterial nanocellulose comprising:
providing a shaped article,
providing a growth medium for bacterial nanocellulose comprising Acetobacteraceae bacteria of the genus Komagataeibacter and the species Komagataeibacter hansenii in the form of a bacterial suspension, and a nutrient solution for said bacteria,
bringing a part of the shaped article into contact with the growth medium for bacterial nanocellulose, and
rotating the shaped article to obtain the shaped element made of bacterial nanocellulose,
wherein rotating the shaped article is carried out at a rotational speed of higher than 10 rpm and maximum 60 rpm and/or wherein the ratio of the bacterial suspension to the nutrient solution is 1:18.

2. The method according to claim 1, wherein the Acetobacteraceae bacteria of the genus Komagataeibacter and the species Komagataeibacter hansenii are Komagataeibacter hansenii with the American Type Culture Collection (ATCC) code 53582.

3-4. (canceled)

5. The method according to claim 1, wherein the nutrient solution comprises at least one monosaccharide and/or one disaccharide, at least one peptone and yeast extract, and wherein the growth medium has an acidic pH value.

6-8. (canceled)

9. The method according to claim 1, wherein the shaped article is made of a polymer selected from a polymer that does not comprise an Si—O group, a polymer that has a polymer backbone containing alternately ketone and ether groups, and a polymer that comprises a polyetheretherketone.

10-12. (canceled)

13. The method according to claim 1, wherein 40% to 60% of a surface of the shaped article is in contact with the growth medium and/or wherein the method is carried out in an oxygen-containing environment.

14. (canceled)

15. The method according to claim 1, wherein rotating the shaped article is carried out at a temperature between 23° C. and 30° C., for 30 h to 114 hours.

16. (canceled)

17. The method according to claim 1, comprising a step of drying the obtained shaped element made of bacterial nanocellulose to obtain a dried shaped element made of bacterial nanocellulose.

18-19. (canceled)

20. The method according to claim 17, wherein the step of drying is carried out in air during the rotation of the shaped article at a rotational speed of less than 10 rpm.

21. The method according to claim 17, wherein, prior to the step of drying, an additional step of treating the obtained bacterial nanocellulose with at least one structure stabilizing agent is carried out to obtain a stabilized shaped element made of bacterial nanocellulose.

22-23. (canceled)

24. The method according to claim 1, wherein the shaped article is a rod, a rotationally symmetric body or is a medical implant and/or the shaped article is covered by a stent, a heart valve prosthesis, a polymer framework, metal framework or metal alloy framework.

25-31. (canceled)

32. An apparatus for producing bacterial nanocellulose comprising:
at least one reactor vessel configured to receive and cultivate a growth medium for bacterial nanocellulose,
a rotating unit configured to drive at least one rotation profile and/or shaped body,
at least one drive unit with a geared motor configured to drive the rotating unit,
at least one gear unit for transmitting the motor torque of the geared motor to the at least one rotary profile and/or the at least one molded body,
at least one detection unit configured to detect a rotational speed of the at least one rotation profile and/or of the at least one shaped body, and
at least one evaluation unit and/or control unit configured to evaluate and/or control rotational speed of the at least one rotation profile and/or of the at least one molded body.

33-35. (canceled)

36. A method for producing bacterial nanocellulose comprising the following steps:
preparing or providing a growth medium for bacterial nanocellulose comprising:
Acetobacteraceae bacteria of the genus Komagataeibacter and the species Komagataeibacter hansenii or K. hansenii and a nutrient solution for said bacteria, wherein the nutrient solution comprises at least one monosaccharide and/or one disaccharide, at least one peptone and yeast extract, and wherein the growth medium has an acidic pH value, and
cultivating the growth medium to obtain bacterial nanocellulose.

37. The method according to claim 36, wherein the Acetobacteraceae bacteria of the genus Komagataeibacter and the species Komagataeibacter hansenii are Komagataeibacter hansenii with the American Type Culture Collection (ATCC) code 53582.

38. The method according to claim 36, wherein the nutrient solution comprises glucose, peptone, yeast extract, disodium hydrogen phosphate, and citric acid or consists of these and/or wherein the Acetobacteraceae bacteria are in a form of a bacterial suspension and the ratio of the bacterial suspension to the nutrient solution is 1:18 and/or wherein the method is carried out in an oxygen-containing environment.

39-40. (canceled)

41. The method according to claim 36, wherein the cultivation of the growth medium is carried out at a temperature between 23° C. and 30° C. for 30-114 hours.

42-44. (canceled)

45. The method according to claim 35, wherein the shaped article comprises or consists of a polymer selected from a polymer that does not comprise Si—O groups, a polymer backbone which contains alternating ketone and ether groups, and polyetheretherketone.

46-49. (canceled)

50. The method according to claim 45, wherein the process further comprises a step of drying the obtained bacterial nanocellulose to obtain a dried bacterial nanocellulose.

51. (canceled)

52. The method according to claim 50, wherein, prior to the step of drying, an additional step of treating the obtained bacterial nanocellulose with at least one structure stabilizing agent is carried out to obtain a stabilized bacterial nanocellulose.

53-54. (canceled)

55. The method according to claim 36, wherein the method further comprises a step of pressing the bacterial nanocellulose before, during or after the step of drying, to obtain a pressed bacterial nanocellulose.

56-58. (canceled)

59. The method according to claim 50 wherein the method further comprises a step of rehydration after drying and/or pressing.

60-72. (canceled)
